SEQUENCE LISTING (1 sequence)

SEQ ID NO: 1 - protein, 19 aa, Artificial Sequence
  fluorescent p53 peptide probe
GSGSSQETFSDLWKLLPEN

(19) United States
(12) Patent Application Publication
(10) Pub. No.: US 2024/0279845 A1
(43) Pub. Date: Aug. 22, 2024

(54) METHOD FOR PREPARING A LIBRARY OF PEPTIDES OR A PEPTIDE

(71) Applicant: ECOLE POLYTECHNIQUE FEDERALE DE LAUSANNE (EPFL), Lausanne (CH)

(72) Inventors: Christian HEINIS, Bern (CH); Sevan HABESHIAN, Renens (CH); Ganesh Kumar MOTHUKURI, Geneve (CH); Mischa SCHUTTEL, Ins (CH); Manuel MERZ, Fribourg (CH); Gontran SANGOUARD, Chavannes-Renens (CH); Zsolt BOGNAR, Morges (CH); Alexander Lund NIELSEN, Lausanne (CH)

(21) Appl. No.: 18/561,248

(22) PCT Filed: Apr. 27, 2022
(86) PCT No.: PCT/EP2022/061138

§ 371 Date: Nov. 15, 2023

(30) Foreign Application Priority Data
May 17, 2021 (EP) 21174036.0

(51) Int. Cl. C40B 50/18; C07K 1/04

(52) U.S. Cl. CPC C40B 50/18; C07K 1/042; C07K 1/047

(57) ABSTRACT

The present invention relates to a method for preparing a library of peptides or an isolated peptide comprising (a) releasing one or more linear dithiol peptides carrying a sulfhydryl group in the N-terminal region of the one or more peptides and being immobilized via a disulfide bridge in the C-terminal region of the one or more peptides on a solid phase from the solid phase by (i) an agent reducing the disulfide bridge, thereby releasing the one or more linear dithiol peptides from the solid phase, wherein the agent is volatile and is removable by evaporation, or (ii) a base that deprotonates the sulfhydryl group in the N-terminal region of the one or more linear dithiol peptides, thereby inducing an intramolecular disulfide exchange thereby releasing the one or more linear dithiol peptides from the solid phase in the form of one or more cyclic peptides.

The present invention relates to a method for preparing a library of peptides or an isolated peptide comprising (a) releasing one or more linear dithiol peptides carrying a sulfhydryl group in the N-terminal region of the one or more peptides and being immobilized via a disulfide bridge in the C-terminal region of the one or more peptides on a solid phase from the solid phase by (i) an agent reducing the disulfide bridge, thereby releasing the one or more linear dithiol peptides from the solid phase, wherein the agent is volatile and is removable by evaporation, or (ii) a base that deprotonates the sulfhydryl group in the N-terminal region of the one or more linear dithiol peptides, thereby inducing an intramolecular disulfide exchange thereby releasing the one or more linear dithiol peptides from the solid phase in the form of one or more cyclic peptides.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Peptide libraries and in particular libraries of cyclic peptides (a class of macrocyclic molecules) have received much interest by the pharmaceutical industry because they can be screened for peptides having the ability to bind to challenging targets, for which it has been difficult or even impossible to generate ligands based on classical small molecules.

Currently, more than 40 cyclic peptides are approved as drugs and more than 100 are being evaluated at different stages in clinical trials (A. Zorzi et al., Curr. Opin. Chem. Biol., 2017, 38, 24-29). Innovative strategies to design cyclic peptide ligands based on protein epitopes or to isolate target-specific cyclic peptides from large combinatorial libraries of genetically encoded peptides have added an additional spin to the development of the field (A. Luther et al., Curr. Opin. Chem. Biol., 2017, 38, 45-51; C. Sohrabi et al., Nat. Rev. Chem., 2020, 4, 90-101).

Of particular interest are macrocyclic molecules that have a sufficiently small size, ideally well below one kilodalton (kDA), and a small polar surface area, that places intracellular targets within reach. Macrocyclic molecules of high interest include small cyclic peptides, macrocyclic structures not based on peptides, or macrocyclic structures containing peptide and non-peptide components. However, the development of macrocyclic ligands binding to targets of interest, and being membrane permeable to reach intracellular targets, is difficult because of the relatively small number of macrocyclic compounds in collections that are commercially provided, or the lack of methods to efficiently synthesize new macrocycle libraries.

A difficult step in the synthesis of macrocyclic compounds is the transformation of linear molecules into cyclic ones. Most reactions offer macrocyclization yields far below 90% and show large variations of the yields for different precursors (e.g. different linear peptide sequences), which is a problem in the synthesis of libraries. Macrocyclic compound libraries offered by leading providers such as Asinex, ChemBridge, or Polyphor are all based on molecules that were individually purified after the macrocyclization step, as the products without purification would not be pure enough for most compounds. The need for purification limits the number of macrocycles that can be produced in parallel and thus the library sizes, which are below 30,000 molecules for commercially offered libraries.

A macrocyclization reaction that generally shows high cyclization yields for a wide substrate range, typically above 90%, is the cyclization of peptides via two thiol groups placed at distant ends in the peptides by bis-elecrophilic reagents, such as bis-(bromomethyl)benzenes (S_{N}2 reaction), bis-(bromoacetamide)benzenes (S_{N}2 reaction), haloacetones (S_{N}2 reaction), vinylsufoxides (Michael addition) or hexafluorobenzene (S_{N}Ar reaction) (H. Jo et al., J. Am. Chem. Soc., 2012, 134, 17704-17713; P. Timmerman et al., ChemBioChem, 2005, 6, 821-824; N. Assem et al., Angew. Chemie-Int. Ed., 2015, 54, 8665-8668; S. S. Kale et al., Nat. Chem., 2018, 10, 715-723). Such reactions were used to develop (bi)cyclic peptides that mimic linear or non-linear epitopes (P. Timmerman et al., ChemBioChem, 2005, 6, 821-824), to evolve (bi)cyclic peptides by phage display (C. Heinis et al., Nat. Chem. Biol., 2009, 5, 502-507) or other display techniques, to stabilize α-helical peptides in helical conformations (H. Jo et al., J. Am. Chem. Soc., 2012, 134, 17704-17713), or to cyclize peptides for other purposes.

Recently large libraries of cyclic peptides were synthesized, having a molecular weight below one kilodalton, by combinatorially cyclizing large numbers of linear dithiol peptides by around 20 different bis-electrophile reagents (Unpublished results of the Laboratory of Therapeutic Proteins and Peptides, EPFL, Group Heinis). The different combinations of dithiol peptides and cyclization reagents were mixed in distinct wells of 384-microwell plates. The high cyclization yields allowed us to screen the cyclic peptides for protein target engagement without prior purification. The omission of a throughput-limiting purification step enabled the screening of large libraries.

The strategy of combinatorial peptide cyclization using bis-electrophile reagents requires a large number of dithiol peptides having different sequences.

Independent of the method used to identify a lead peptide, the development of peptide libraries and peptide drugs typically involves multiple iterative cycles of synthesizing dozens to hundreds of peptide variants to improve key properties such as binding affinity, specificity, stability, pharmacokinetic properties and others, and thus the preparation of large numbers of peptides.

A further major bottleneck in the development of cyclic peptide therapeutics is the chromatographic purification of peptides after synthesis that is expensive due to the sequential processing of each peptide and high reagent consumption (solvents), even when automated and optimized. The main reason for the need of purifications is the macrocyclization reaction, being typically the most difficult step in cyclic peptides synthesis (C. J. White et al., Nat. Chem., 2011, 3, 509-524). Most macrocyclization reactions show yields below 90% and the efficiencies often vary strongly depending on the peptide sequence and length. Chromatographic purification is also required to remove reagents and scavengers added for peptide release, as well as side chain protecting group byproducts generated during global deprotection.

It is evident from the above that there is an urgent need for novel methods, in particular methods that require less sophisticated steps of preparation for the generation of large peptide libraries or one or more peptides that can be added to peptide libraries, wherein the peptide libraries may then be screened for peptide therapeutics. This need is addressed by the present invention.

The present invention relates in a first aspect to a method for preparing a library of peptides or an isolated peptide comprising (a) releasing one or more linear dithiol peptides carrying a sulfhydryl group in the N-terminal region of the one or more peptides and being immobilized via a disulfide bridge in the C-terminal region of the one or more peptides on a solid phase from the solid phase by (i) an agent reducing the disulfide bridge, thereby releasing the one or more linear dithiol peptides from the solid phase, wherein the agent is volatile and can is removable by evaporation, or (ii) a base that deprotonates the sulfhydryl group in the N-terminal region of the one or more linear dithiol peptides, thereby inducing an intramolecular disulfide exchange thereby releasing the one or more linear dithiol peptides from the solid phase in the form of one or more cyclic peptides.

The term “comprise/comprising” is generally used in the sense of include/including, that is to say permitting the presence of one or more features or components. The terms “comprise” and “comprising” also encompass the more restricted terms “consist of” and “consisting of”.

As used in the specification and claims, the singular form “a”, “an” and “the” include plural references unless the context clearly dictates otherwise.

The term “peptide” as used herein refers to a polymer comprising at least one amino acid and at least one peptide bond. The peptide preferably comprises multiple—such as two or more, three or more, four or more, or five or more—amino acids. The peptide may also comprise non-amino acid building blocks and non-peptide linkages. Many of the peptides as described in the appended examples contain at their termini building blocks such as mercaptopropanoic acids (MPA) and cysteamine (MEA) that are not amino acids as they do not contain an amino group (in the case of MPA) or not a carboxylic acid (in the case of MEA).

Further examples of non-amino acid building blocks that contain a thiol group and are suitable for incorporation into peptides, in particular at the C-terminal end (as MEA) are 3-aminopropane-1-thiol, 3-(methylamino)propane-1-thiol, (Z)-4-aminobut-2-ene-1-thiol, piperidine-4-thiol, 4-(mercaptomethyl)piperidine, and 3-(mercaptomethyl)azetidine; see below table and formulas. Among these non-amino acid building blocks, those with secondary amines are particularly preferred for developing membrane-permeable macrocyclic compounds as the coupling of a secondary amine and a carboxylic acid (of the neighboring amino acid) yields an amide bond without a hydrogen-bond donor group.

| Number | Name |
|---|---|
| 1 | 3-aminopropane-1-thiol |
| 2 | 3-(methylamino)propane-1-thiol |
| 3 | (Z)-4-aminobut-2-ene-1-thiol |
| 4 | piperidine-4-thiol |
| 5 | 4-(mercaptomethyl)piperidine |
| 6 | 3-(mercaptomethyl)azetidine |

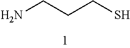

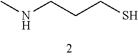

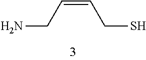

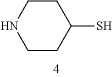

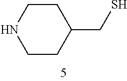

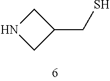

Further examples of non-amino acid building blocks that contain a thiol group and are suitable for incorporation into the peptides, in particular at the N-terminal end (as MPA) are 2-mercaptoacetic acid, (R)-2-mercaptopropanoic acid, 5-(mercaptomethyl)furan-2-carboxylic acid, and 3-(mercaptomethyl)benzoic acid; see below table and formulas.

| Number | Name |
|---|---|
| 1 | 2-mercaptoacetic acid |
| 2 | (R)-2-mercaptopropanoic acid |
| 3 | 5-(mercaptomethyl)furan-2-carboxylic acid |
| 4 | 3-(mercaptomethyl)benzoic acid |

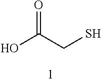

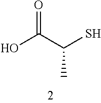

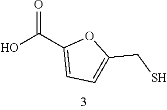

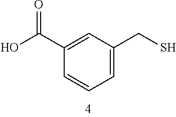 [SMILES: O=C(O)c1cccc(CS)c1]

Various other non-amino acid building blocks are suitable for incorporation into the dithiol peptides, in particular at internal positions. For example, a building block A-COOH can be coupled to an amino group of the previous residue on the solid phase and a building block B-NH₂ can next be appended, wherein the functional groups in A and B react with each other to form a covalent bond. This latter reaction can yield a linkage that is not a peptide bond. An example of such a reaction is the established, so-called “sub-monomer” strategy in which haloacetic acid, typically bromoacetic acid that is activated (e.g. by diisopropylcarbodiimide), is first coupled to the amino group of a growing peptide on solid phase (the haloacetic acid representing the building block A-COOH). In a second step and chemical reaction, an amine (representing the B-NH₂) displaces the halide to form an N-substituted glycine residue (by a classical SN₂ reaction). The sub-monomer approach allows the use of any commercially available or synthetically accessible amine which is of great advantage as many amines can be used in parallel and a large peptide diversity can be generated. Instead of the haloacetic acids, many other reagents can be used, including as preferred examples 4-(bromomethyl)benzoic acid, 3-(bromomethyl)benzoic acid, 2-(chloromethyl)oxazole-4-carboxylic acid, 2-(chloromethyl)thiazole-4-carboxylic acid, 5-(bromomethyl)isoxazole-3-carboxylic acid, 5-(bromomethyl)pyrazine-2-carboxylic acid, 2-(bromomethyl)furan-3-carboxylic acid, (R,E)-5-chloro-2,4-dimethylpent-3-enoic acid, and (S,E)-5-chloro-2,4-dimethylpent-3-enoic acid; see below table and formulas.

| Number | Name |
|---|---|
| 1 | 4-(bromomethyl)benzoic acid |
| 2 | 3-(bromomethyl)benzoic acid |
| 3 | 2-(chloromethyl)oxazole-4-carboxylic acid |
| 4 | 2-(chloromethyl)thiazole-4-carboxylic acid |
| 5 | 5-(bromomethyl)isoxazole-3-carboxylic acid |
| 6 | 5-(bromomethyl)pyrazine-2-carboxylic acid |
| 7 | 2-(bromomethyl)furan-3-carboxylic acid |
| 8 | (R,E)-5-chloro-2,4-dimethylpent-3-enoic acid |
| 9 | (S,E)-5-chloro-2,4-dimethylpent-3-enoic acid |

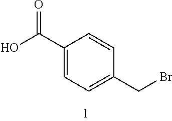 [SMILES: O=C(O)c1ccc(CBr)cc1]

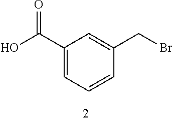 [SMILES: O=C(O)c1cccc(CBr)c1]

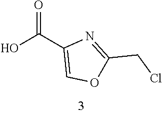

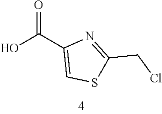

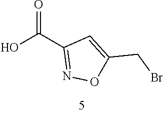

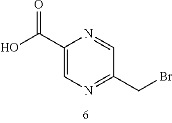 [SMILES: O=C(O)c1cnc(CBr)cn1]

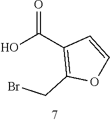

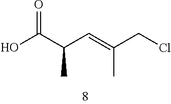

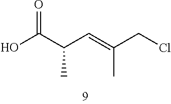

The term “peptide” preferably designates short chains of amino acids linked by peptide bonds. Also the short chains of amino acids linked by peptide bonds may at their termini contain non-amino acid building blocks, such as MPA and cysteamine MEA. Peptides are distinguished from proteins or polypeptides on the basis of size, and comprise with increasing preference less than 50 amino acids and with increasing preference less than 40 amino acids, less than 30 amino acids, less than 20 amino acids, less than 10 amino acids and less than 5 amino acids.

The term “isolated peptide” as used herein is to indicate that the peptide is not bound to the solid phase but has been released from the solid phase. The isolated peptide is generally in the form of a free peptide, for example, in solution. In the solution, one or several copies of the isolated peptide may be present. Accordingly, also on the solid phase one or several copies of the peptide to be isolated may be present before the peptide is.

The term “amino acid” as used herein refers to an organic compound composed of amine (—NH₂ or —NH) and carboxylic acid (—COOH) functional groups, generally along with a side-chain specific to each amino acid. The simplest amino acid glycine does not have a side chain (formula H₂NCH₂COOH). In amino acids that have a carbon chain attached to the α-carbon (such as lysine), the carbons are labelled in the order α, β, γ, δ, and so on. In some amino acids, the amine group may be attached, for instance, to the α-, β- or γ-carbon, and these are therefore referred to as α-, β- or γ-amino acids, respectively. The term “amino acid” preferably describes α-amino acids (also designated 2-, or alpha-amino acids) which generally have the generic formula H₂NCHRCOOH, wherein R is an organic substituent being designated “side-chain”) and also beta-, gamma- and delta-amino acids, that contain multiple carbon atoms between the amine and the carboxylic acid. In the simplest α-amino acid alanine (formula: H₂NCHCH₃COOH) the side is a methyl group. The amino acids are in accordance with the present invention are L-amino acids or D-amino acids.

The amino acids comprise the so-called standard or canonical amino acids. These 21 α-amino acids are encoded directly by the codons of the universal genetic code. They are the proteinogenic α-amino acids found in eukaryotes. These amino acids are referred to herein in the so-called one-letter code:

| | | | |
|---|---|---|---|
| G | Glycine | P | Proline |
| A | Alanine | V | Valine |
| L | Leucine | I | Isoleucine |
| M | Methionine | C | Cysteine |
| F | Phenylalanine | Y | Tyrosine |
| W | Tryptophan | H | Histidine |
| K | Lysine | R | Arginine |
| Q | Glutamine | N | Asparagine |
| E | Glutamic Acid | D | Aspartic Acid |
| S | Serine | T | Threonine |
| U | Selenocysteine | | |

As mentioned, the side-chain of an amino acid is an organic substituent, which is in the case of α-amino acids linked to the α-carbon atom. Hence, a side chain is a branch from the parent structure of the amino acid. Amino acids are usually classified by the properties of their side-chain. For example, the side-chain can make an amino acid a weak acid (e.g. amino acids D and E) or a weak base (e.g. amino acids K and R), and a hydrophile if the side-chain is polar (e.g. amino acids L and I) or a hydrophobe if it is non-polar (e.g. amino acids S and C). An aliphatic amino acid has a side chain being an aliphatic group. Aliphatic groups render the amino acid nonpolar and hydrophobic. The aliphatic group is preferably an unsubstituted branched or linear alkyl. Non-limiting examples of aliphatic amino acids are A, V, L, and I. In a cyclic amino acid one or more series of atoms in the side chain is/are connected to form a ring. Non-limiting examples of cyclic amino acids are P, F, W, Y and H. It is to be understood that said ring has to be held distinct from the ring that is formed in case of cyclic peptide as will be further detailed herein below. While the former ring of a cyclic amino acid is a part of the side chain of a single amino acid the latter ring is formed between to two thiol groups of a dithiol peptide. An aromatic amino acid is the preferred form of a cyclic amino acid. In an aromatic amino acid the ring is an aromatic ring. In terms of the electronic nature of the molecule, aromaticity describes the way a conjugated ring of unsaturated bonds, lone pairs of electrons, or empty molecular orbitals exhibits a stronger stabilization than would be expected by the stabilization of conjugation alone. Aromaticity can be considered a manifestation of cyclic delocalization and of resonance. A hydrophobic amino acid has a non-polar side chain making the amino acid hydrophobic. Non-limiting examples of hydrophobic amino acids are M, P, F, W, G, A, V, L and I. A polar, uncharged amino acid has a non-polar side chain with no charged residues. Non-limiting examples of polar, uncharged amino acids are S, T, N, Q, C, U and Y. A polar, charged amino acid has a non-polar side chain with at least one charged residue. Non-limiting examples of polar, charged amino acids are D, E, H, K and R.

A “dithiol peptide” is a peptide that contains two or more and preferably only two thiol groups. The thiol groups are parts of either amino acids or non-amino acid building blocks of the peptide. The amino acid or non-amino acid building blocks carrying a thiol group can be one of the following:
A cysteine or cysteine analogue, such as homocysteine, penicillamine or D-cysteine
A building bock containing a thiol group and a carboxylic acid, as for example mercaptopropanoic acid (MPA)
A building bock containing a thiol group and an amine, as for example cysteamine (MEA)

When the linear dithiol peptide is immobilized on a solid phase, one thiol group is “free”, initially a protected sulfhydryl group (R-S-PG; PG=protecting group), and after removal of the thiol-protecting group a sulfhydryl group (R-SH), while the other thiol group is engaged in a disulfide bond (R¹-S—S-R²) connecting the peptide to the solid phase. A disulfide bridge is created when a sulfur atom from one thiol-containing building block (linked to the solid phase) forms a single covalent bond with a sulfur atom from a thiol-containing building block in the C-terminal region of the peptide. Means and methods for synthesizing peptides via disulfide bonds to a solid phase are known in the art, further detailed herein below and illustrated by the appended examples.

The term “linear peptide” designates a linear short chain of amino acids linked by peptide bonds that can contain also non-amino acids and non-peptide bonds as described herein above connection with the “peptide” according to the invention. In the linear peptides, no series of atoms in the peptide is/are connected to form a macrocyclic ring or cycle.

The term “cyclic peptide” means that a series of 12 or more atoms in the peptide is connected to form a macrocyclic ring or cycle. The cyclic peptide is preferably a monocyclic peptide which means that only two sites in the peptide is/are connected to form a ring or cycle. The ring or cycle is formed in accordance with the invention by involving the two thiol groups of the dithiol peptides. In case the two thiol groups are directly bonded, the ring or cycle is a formed by a disulfide bridge. The two thiol groups of the dithiol peptides may also be connected by a bis-electrophilic reagent, as will be explained in more detail herein below.

A library of peptides refers to a composition or an article of manufacture comprising a plurality of different peptides.

In the case of the library being a composition, the composition comprises a mixture of different peptides.

The composition is preferably a solution and more preferably a DMSO or an aqueous solution. The solution can be dried, for example, by lyophilization or centrifugal vacuum evaporation (e.g. using a SpeedVac system). In this case the composition can be in the form of a dried powder that can be dissolved by a desirable solvent. In the case of such a composition the number of different peptides in the library can be determined by the number of different peptides being immobilized on the solid support and/or by mixing solid support onto which one, or two or more different peptides were synthesized.

The article of manufacture comprises different wells and is preferably a microtiter plate, more preferably a 96-well plate, a 384-well plate, a 1536-well plate or a 3456-well plate. The different peptides are preferably synthesized (generally on a solid support) in parallel in the different wells, so that one kind or peptide (in many copies) can be found per well. The plurality of wells of the article of manufacture together forms the library of peptides. A library in the format of an article of manufacture is preferred since in case such library is screened for a binder to or inhibitor of a particular target molecule (as described herein below) it is immediately known in which well the desired library member can be found and no further complex isolation or identification of the desired library member is needed.

While one kind of peptide per well is preferred it is also possible to synthesize more than one peptide, such as two, three, four or five different peptides per well (generally on a solid support) in parallel in each wells, thereby obtaining wells with two, three, four or five different peptides per well. The number of different peptides per well can be adjusted as needed, for example, by the number of different peptides being immobilized on the solid support and/or by mixing solid supports onto which one, or two or more different peptides were synthesized.

The library of different peptides comprises with increasing preference at least 2, at least 3, at least 4, at least 5, at least 10, at least 20, at least 50, at least 96, at least 100, at least 384, at least 500, at least 1000, at least 1536, at least 3456, at least 10000, and at least 100000 different peptides.

The N-terminal region preferably designates one of the most three N-terminal amino acids or building blocks, more preferably one of the most two N-terminal amino acids or building blocks and most preferably the most N-terminal amino acid or building blocks of the one or more linear dithiol peptides. In the most preferred case, the sulfhydryl group is part of an amino acid or building block at the N-terminal position of the one or more linear dithiol peptides.

Likewise, the C-terminal region preferably designates one of the most three C-terminal amino acids or building blocks, more preferably one of the most two C-terminal amino acids or building blocks and most preferably the most C-terminal amino acid or building block of one or more linear dithiol peptides. In the most preferred case, the one or more peptides are immobilized via a disulfide bridge at the C-terminus to a solid phase.

The term “solid phase” designates any solid material or support to which peptides can be synthesized via a disulfide bond, e.g. via solid-phase peptide synthesis (SPPS).

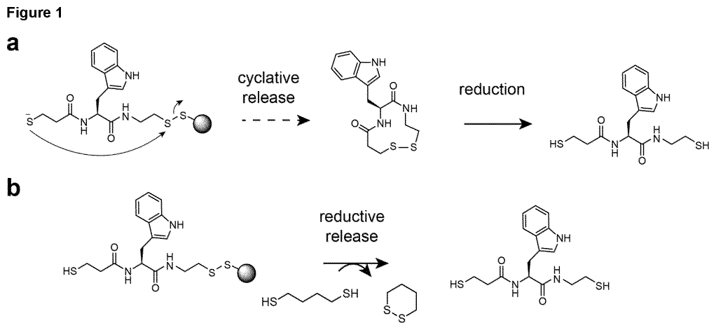

The term “agent”, as used herein, designates any molecule being capable of reducing the disulfide bridge, thereby releasing the one or more linear dithiol peptides from the solid phase. The agent may therefore also be designated as a reductant or releasing agent. A variety of reductants are known in the prior art. In biochemistry, thiols such as β-mercaptoethanol (β-ME) or dithiothreitol (DTT) serve as reductants. In accordance with the invention the agent is volatile and is removable by evaporation, preferably by evaporation under vacuum and most preferably by centrifugal vacuum evaporation. A volatile substance will change easily into a gas by evaporation. Evaporation is a type of vaporization that occurs on the surface of a liquid as it changes into the gas phase. The ability for a molecule of a liquid to evaporate is based largely on the amount of kinetic energy an individual particle may possess. While the evaporation rate increases at higher temperatures, even at lower temperatures individual molecules of a liquid can evaporate if they have more than the minimum amount of kinetic energy required for vaporization. The use of an agent that is volatile and is removable by evaporation is technically advantageous because the agent can be removed from the composition comprising the library of peptides or the isolated peptide to be produced by the method of the invention. The removal of the one or more peptides from the solid support by an agent is also designated “reductive release” herein and is illustrated by FIG. 1b. As can be taken from FIG. 1b the one or more peptides are released by the agent in the form of one or more linear peptides, wherein the two thiol groups are “free” sulfhydryl groups.

A base is a substance which can accept protons (such as Brønsted bases), donate electrons (such as Lewis bases), or any chemical compound that yields hydroxide ions (OH—) in aqueous solution. The base used in accordance with the invention is capable of deprotonating the sulfhydryl group in the N-terminal region of the one or more linear dithiol peptides (R-S⁻). The deprotonated sulfhydryl group induces an intramolecular disulfide exchange whereby the one or more linear dithiol peptides are released from the solid phase in the form of one or more cyclic peptides. The deprotonated sulfhydryl group comprises a reactive S that acts as a nucleophilic agent inducing a thiol-disulfide exchange reaction at the disulfide bond. The removal of the one or more peptides from the solid support by a base is also designated “cyclative release” herein and is illustrated by FIG. 1a. As can be taken from FIG. 1a the one or more peptides are released by the base in the form of a cyclic peptide, wherein the two thiol groups of the dithiol peptide are connected by a disulfide bond.

As discussed above, the method of claim 1, item (i) is a “reductive release” approach resulting in linear dithiol peptides while the method of claim 1, item (ii) is a “cyclative release” approach resulting in cyclic dithiol peptides. As will become evident from the following, both options significantly advance the prior art methods for preparing a library of peptides or an isolated peptide.

The “reductive release” strategy in which the dithiol peptides being immobilized via a disulfide bridge on a solid support are released from the solid phase by disulfide reduction from the resin as schematically shown in FIG. 1b. In contrast to the cyclative release, the efficiency of the reductive release is independent of the length and amino acid composition of the peptides. In particular, this approach also efficiently works with short peptides that cannot efficiently cyclize via disulfide formation (but can be cyclized via bis-electrophile linkers as this cyclization is sterically less demanding due to the additional atoms added by the linker to the macrocycle backbone). A challenge in the reductive peptide release was that a reducing agent needs to be added to the peptide, and this reagent interferes with the subsequent cyclization reaction by bis-electrophile reagents (i.e. react with the electrophilic groups). This drawback is overcome by using a volatile reducing agent that is removable from the peptides by evaporation, for example, under vacuum in a relatively easy step. For example, the volatile reducing agent can be removed by centrifugal vacuum evaporation of the peptides in 96-well plates.

A handful of studies describe the release of peptides from solid phase by reductively breaking a disulfide bridge, all of them releasing peptides having a single thiol group and having other applications in mind such as the generation of peptide-protein conjugates (J. Mery et al., Int. J. Pept. Protein Res., 1993, 42, 44-52), the synthesis of peptide heterodimers (A. Taguchi et al., Org. Biomol. Chem., 2015, 13, 3186-3189), the production of head-to-tail cyclized peptides containing a thiol handle (W. Tegge et al., J. Pept. Sci., 2007, 13, 693-699), the cyclization of peptides via one thiol group (A. A. Virgilio et al., Tetrahedron Lett., 1996, 37, 6961-6964), the identification of released peptides by mass spectrometry (O. Lack et al., Helv. Chim. Acta, 2002, 85, 495-501), and the transient immobilization during peptide synthesis (D. S. Kemp et al., J. Org. Chem., 1986, 51, 1821-1829). Most of these approaches used the reducing agents tris-(2-carboxyethyl)phosphine (TCEP) (J. Mery et al., Int. J. Pept. Protein Res., 1993, 42, 44-52; A. A. Virgilio et al., Tetrahedron Lett., 1996, 37, 6961-6964), dithiothreitol (DTT) (J. Mery et al., Int. J. Pept. Protein Res., 1993, 42, 44-52; W. Tegge et al., J. Pept. Sci., 2007, 13, 693-699) and tri-n-butylphosphine (P(n-Bu)₃) (D. S. Kemp et al., J. Org. Chem., 1986, 51, 1821-1829), and thus reagents that cannot be removed by evaporation, and require a purification step. In only one of these studies, a volatile reducing agent, β-mercaptoethanol (β-Me), was used for the release of disulfide-linked peptides from the solid support (O. Lack et al., Helv. Chim. Acta, 2002, 85, 495-501). However, in this application, the peptides were released from single beads and in small quantities from a resin that was polar (PEGA), has a low loading (0.2 mmol/gram), and a high swelling volume, which was not suited for the presently envisioned application. None of the studies reported the synthesis and reductive release of dithiol peptides, and none of the studies applied the approach for the generation of cyclic peptide libraries.

To the best knowledge of the inventors, the strategy of cyclative disulfide release is completely new and has not been used for the generation of cyclic peptides. Strategies that come closest to the cyclative release are the oxidative release of thioether-immobilized peptides (B. H. Rietman et al., Int. J. Pept. Protein Res., 1994, 44, 199-206; T. Zoller et al., Tetrahedron Lett., 2000, 41, 9989-9992). However, these approaches are not suited for library generation due to the low yields, dimeric side products and the presence of oxidants in the eluted product that would need removal by purification. Herein, the cyclative release is triggered by a base that deprotonates the N-terminal sulfhydryl group. If using a volatile base it could be removed by evaporation so that the only product in the reaction tube or microtiter plate well is one or more pure cyclic peptides. If using a non-volatile base, it could also be neutralized by an acid. An important requirement for the cyclative strategy was that peptides can be synthesized on solid phase immobilized via a disulfide bridge, wherein the disulfide bridge needed to be sufficiently stable during the peptide synthesis and in particular during Fmoc deprotection by piperidine. Several studies reported the solid-phase synthesis of disulfide bridge-immobilized peptides for applications ranging from the generation of peptide-protein conjugates to the reductive release of peptides for mass spectrometric identification (J. Mery et al., Int. J. Pept. Protein Res., 1993, 42, 44-52; A. Taguchi et al., Org. Biomol. Chem., 2015, 13, 3186-3189; W. Tegge et al., J. Pept. Sci., 2007, 13, 693-699; A. A. Virgilio et al., Tetrahedron Lett., 1996, 37, 6961-6964; O. Lack et al., Helv. Chim. Acta, 2002, 85, 495-501; D. S. Kemp et al., J. Org. Chem., 1986, 51, 1821-1829). Mery, J. and co-workers reported that the stability of the disulfide-bridge depends on substituents on the carbon atoms next to the sulfurs and the linker NH₂—CH₂—CH₂—S—S—C(CH₃)₂—COOH with bulky methyl groups, which was sufficiently stable for Fmoc peptide synthesis (J. Mery et al., Int. J. Pept. Protein Res., 1993, 42, 44-52; W. Tegge et al., J. Pept. Sci., 2007, 13, 693-699; J. Mery et al., Pept. Res., 1992, 5, 233-240). Previously peptides disulfide-immobilized via the disulfide linker NH₂—CH₂—CH₂—S—S—CH₂—C(NH₂)H—COOH were synthesized and it was found that no substantial amount of peptide in the case of short peptides, despite the rather unhindered linker, probably due to the limited number of times the beads are exposed to piperidine (Y. Wu et al., Chem. Commun., 2020, 56, 2917-2920).

In accordance with a preferred embodiment of the first aspect of the invention the method comprises after step (a) the step of removing the agent by evaporation.

As discussed above, the agent to be used in the claimed is volatile and is removable by evaporation. According to this preferred embodiment the step of removing the agent by evaporation forms part of the method. The evaporation is preferably conducted under vacuum (e.g. using a SpeedVac system). The agent is most preferably removed by centrifugal vacuum evaporation.

Also the base according to the invention is preferably volatile and is removable by evaporation. In this case the method of the first aspect of the invention preferably comprises after step (a) the step of removing the base by evaporation.

As an alternative of removing the agent by evaporation, the agent can also be removed by lyophilization.

In accordance with another preferred embodiment of the first aspect of the invention the method further comprises step (b) of cyclizing the one or more linear dithiol peptides being released by the agent.

As discussed above, in the case of the “reductive release” by the agent the one or more peptides are released by the agent in the form of linear peptides, wherein the two thiol groups are “free” sulfhydryl groups. The two sulfhydryl groups can be used to cyclize the peptides.

In accordance with a more preferred embodiment of the first aspect of the invention the one or more dithiol peptides are cyclized by at least one bis-electrophilic reagent or by disulfide oxidation.

The two sulfhydryl groups can either be directly connected to form a disulfide bond (disulfide oxidation) or via a connecting molecule, in particular via a bis-electrophilic reagent. A range of different bis-electrophilic reagents are commercially available, or they can easily be synthesized by routine chemical reactions. Different bis-electrophilic reagents can be used in parallel reactions to produce many different cyclic peptides from one dithiol peptide.

Electrophilic reagents act as electron-pair acceptors in the formation of a new bond. In the case of nucleophilic substitutions, a leaving group departs as a negatively charged species. The bis-electrophilic reagent is a chemical compound comprising at least two functional groups that can be reacted with the two sulfhydryl groups, whereby sulfhydryl groups are connected via the bis-electrophilic reagent.

A disulfide oxidation results in a direct disulfide bond connecting the two sulfhydryl groups of a dithiol peptide.

In accordance with another preferred embodiment of the first aspect of the invention the disulfide bonds of the one or more cyclic peptides of item (ii) are reduced and the peptides are recyclized by a bis-electrophilic reagent.

As discussed herein above, in accordance with item (ii) of the first aspect of the invention the one or more peptides are released by the base in the form of a cyclic peptide, wherein the two thiol groups of the dithiol peptide are connected by disulfide bonds.

These disulfide bonds can be reduced, preferably by an agent as described in connection with item (i) of the first aspect of the invention, so that linear peptides with two “free” sulfhydryl groups are obtained. These linear peptides can then be recyclized by a bis-electrophilic reagent as explained herein above.

In accordance with a further preferred embodiment of the first aspect of the invention the agent is selected from 1,3-propanedithiol, 1,4-butanedithiol, 2,4-pentanedithiol, ethane-1-thiol, propane-1-thiol, butane-1-thiol, propane-2-thiol, 2-methyl-1-propanethiol, butane-2-thiol, 2-methylpropane-2-thiol, 2-hydroxy-1-ethanethiol, 1,2-ethanedithiol, 2-propene-1-thiol, 3-methyl-1-butanethiol, thiophenol, benzylthiol, 2-butene-1-thiol, 3-butene-1-thiol, 2-methyl-2-propene-1-thiol and 3-methyl-2-butene-1-thiol, and is preferably 1,3-propanedithiol, 1,4-butanedithiol or 2,4-pentanedithiol and is most preferably 1,4-butanedithiol (BDT) and/or the base is selected from a tertiary, secondary or primary amine, a boron, aluminium or silicon hydride, and a base with an oxygen, nitrogen or carbon anion, and is preferably a tertiary, secondary or primary amine, more preferably a tertiary amine, even more preferably a tri-alkyl amine and most preferably N,N-diisopropylethylamine (DIPEA).

Preferred examples of reducing agents that are removable by evaporation are shown in Table 1.

TABLE 1

| | | Boiling point (° C.) |
|---|---|---|
| Number | Name | (760 mm Hg) |
| 1* | 1,3-propanedithiol | 169 |
| 2* | 1,4-butanedithiol | 195.5 |
| 3* | 2,4-pentanedithiol | 194 |
| 4 | ethane-1-thiol | 35 |
| 5 | propane-1-thiol | 68 |
| 6 | butane-1-thiol | 98.2 |
| 7 | propane-2-thiol | 126.6 |
| 8 | 2-methyl-1-propanethiol | 88.5 |
| 9 | butane-2-thiol | 84.5 |
| 10 | 2-methylpropane-2-thiol | 64 |
| 11 | 2-hydroxy-1-ethanethiol | 157 |
| 12 | 1,2-ethanedithiol | 146 |
| 13 | 2-propene-1-thiol | 65 |
| 14 | 3-methyl-1-butanethiol | 120 |
| 15 | thiophenol | 169 |
| 16 | benzylthiol | 194.5 |
| 17 | 2-butene-1-thiol | 101 |
| 18 | 3-butene-1-thiol | 98 |
| 19 | 2-methyl-2-propene-1-thiol | 93 |
| 20 | 3-methyl-2-butene-1-thiol | 130 |

*) These reducing agents are preferred as they do not generate adducts. The chemical structure of compounds 1 to 20 is

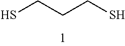

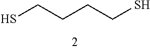

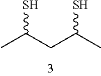

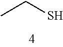

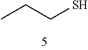

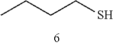

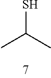

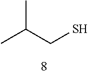

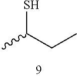

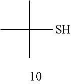

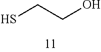

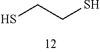

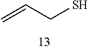

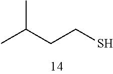

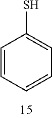

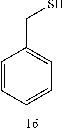

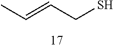

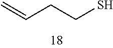

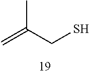

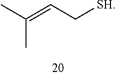

1,4-butanedithiol (BDT) is used as the agent in the appended examples and is therefore the most preferred agent.

Preferred examples of bases for deprotonating sulfhydryl groups to induce disulfide exchange and cyclative peptide release are shown in Table 2.

TABLE 2

| Number | Name | Type | Sub-type |
|---|---|---|---|
| 1 | N,N-diisopropylethylamine | Tertiary amine | Tri-alkyl |
| 2 | Triethylamine | Tertiary amine | Tri-alkyl |
| 3 | Tributylamine | Tertiary amine | Tri-alkyl |
| 4 | Quinuclidine | Tertiary amine | Tri-alkyl |
| 5 | DABCO | Tertiary amine | Tri-alkyl |
| 6 | N-methylpiperidine | Tertiary amine | Tri-alkyl |
| 7 | N-methylmorpholine | Tertiary amine | Tri-alkyl |
| 8 | 1,8-Diazabicyclo[5.4.0]undec-7-ene (DBU) | Tertiary amine | Amidine |
| 9 | 1,5-Diazabicyclo[4.3.0]non-5-ene | Tertiary amine | Amidine |
| 10 | 1,1,3,3-Tetramethylguanidine | Tertiary amine | Guanidine |
| 11 | 7-Methyl-1,5,7-triazabicyclo[4.4.0]dec-5-ene | Tertiary amine | Guanidine |
| 12 | 1,5,7-Triazabicyclo[4.4.0]dec-5-ene | Tertiary amine | Guanidine |
| 13 | Pyridine | Tertiary amine | Heterocyclic |
| 14 | 2,6-Lutidine | Tertiary amine | Heterocyclic |
| 15 | Collidine | Tertiary amine | Heterocyclic |
| 16 | 4-dimethylaminepyridine (DMAP) | Tertiary amine | Heterocyclic |
| 17 | Imidazole | Tertiary amine | Heterocyclic |
| 18 | Dimethylaniline | Tertiary amine | Aromatic |
| 19 | 1,8-Bis(dimethylamino)naphthalene | Tertiary amine | Aromatic |
| 20 | tert-Butylimino-tri(pyrrolidino)phosphorane | Tertiary amine | Phosphazene |
| 21 | Piperidine | Secondary amine | Di-alkyl |
| 22 | Morpholine | Secondary amine | Di-alkyl |
| 23 | Pyrrolidine | Secondary amine | Di-alkyl |
| 24 | N-methylaniline | Secondary amine | Aromatic |
| 25 | Propylamine | Primary amine | Alkyl |
| 26 | Allylamine | Primary amine | Alkyl |
| 27 | Benzylamine | Primary amine | Alkyl |
| 28 | Aniline | Primary amine | Aromatic |
| 29 | Hydride (various cations, such as | Hydride | Alkali metal hydride |
| | Lithium/Sodium/Potassium/Calcium) | | |
| 30 | Borane | Hydride | Boron hydride |
| 31 | Borohydride (various cations) | Hydride | Boron hydride |
| 32 | Cyanoborohydride (various cations) | Hydride | Boron hydride |
| 33 | Triacetoxyborohyride (various cations) | Hydride | Boron hydride |
| 34 | Aluminum hydride (various cations) | Hydride | Aluminum hydride |
| 35 | Diisobutylaluminum hydride | Hydride | Aluminum hydride |
| 36 | Silane | Hydride | Silicon hydride |
| 37 | Triethylsilane | Hydride | Silicon hydride |
| 38 | Hydroxide (various cations) | Oxygen anion | Hydroxide |
| 39 | Alkoxides (various cations) | Oxygen anion | Alkoxide |
| 40 | Phenoxides (various cations) | Oxygen anion | Phenoxide |
| 41 | Diisopropylamide (various cations) | Nitrogen anion | Amide |
| 42 | HMDS (various cations) | Nitrogen anion | Amide |
| 43 | Amide (—NH₂) various cations, such as | Nitrogen anion | Amide |
| | LiNH₂/NaNH₂ | | |
| 44 | N/sec/tert-butyllithium | Carbon anion | Organometallic |
| 45 | N/sec/tert-butylpotassium | Carbon anion | Organometallic |
| 46 | Trialkylaluminum | Carbon anion | Organometallic |
| 47 | Dialkylzinc | Carbon anion | Organometallic |
| 48 | Alkyl/aryl magnesium chloride | Carbon anion | Grignard |

The list in Table 2 is not exhaustive but provides examples of useful reagents of each type/subtype. Preferred are the tri-alkyl tertiary amines and in particular the specific examples thereof. N,N-diisopropylethylamine (DIPEA) is used as the base in the appended examples and is therefore the most preferred base.

In accordance with a further preferred embodiment of the first aspect of the invention the method comprises prior to step (a) the step (a′) of the synthesis of the linear dithiol peptides on the solid phase.

Solid-phase peptide synthesis (SPPS) is a common technique for peptide synthesis. Usually, peptides are synthesised from the carbonyl group side (C-terminus) to amino group side (N-terminus) of the amino acid chain in the SPPS method, although peptides are biologically synthesised in the opposite direction in cells. In peptide synthesis, an amino-protected amino acid is bound to a solid phase material, forming a covalent bond between the carbonyl group and the solid phase material. Then the amino group is deprotected and reacted with the carbonyl group of the next, N-protected, amino acid. The solid phase now bears a dipeptide. This cycle is repeated to form the desired peptide chain.

In accordance with another preferred embodiment of the first aspect of the invention the side chains of the amino acids of the linear dithiol peptides are protected by protecting groups and the method further comprises prior to step (a) and, if present, after (a′) the removal of the protecting groups while the linear dithiol peptides are immobilized on the solid phase.

Because amino acids have multiple reactive groups, peptide synthesis must be carefully performed to avoid side reactions that can reduce the length and cause branching of the peptide chain. To facilitate peptide formation with minimal side reactions, chemical groups have been developed that bind to the amino acid reactive groups and block, or protect, the functional group from nonspecific reaction.

Generally purified, individual amino acids used to synthesize peptides are reacted with these protecting groups prior to synthesis, and then specific protecting groups are removed from the newly added amino acid (a step called deprotection) just after coupling to allow the next incoming amino acid to bind to the growing peptide chain in the proper orientation. Once peptide synthesis is completed, all remaining protecting groups are removed from the nascent peptides.

Three types of protecting groups are generally used in the art, depending on the method of peptide synthesis, and are described below.

The amino acid N-termini are protected by groups that are termed “temporary” protecting groups, because they are relatively easily removed to allow peptide bond formation. Two common N-terminal protecting groups are tert-butoxycarbonyl (Boc) and 9-fluorenylmethoxycarbonyl (Fmoc), and each group has distinct characteristics that determine their use. Boc requires a moderately strong acid such as trifluoracetic acid (TFA) to be removed from the newly added amino acid, while Fmoc is a base-labile protecting group that is removed with a mild base such as piperidine. Boc chemistry requires acidic conditions for deprotection, while Fmoc, which was not reported for another twenty years, is cleaved under mild, basic conditions. Because of the mild deprotection conditions, Fmoc chemistry is more commonly used in commercial settings because of the higher quality and greater yield, while Boc is preferred for complex peptide synthesis or when non-natural peptides or analogs that are base-sensitive are required.

The use of a C-terminal protecting group depends on the type of peptide synthesis used; while liquid-phase peptide synthesis requires protection of the C-terminus of the first amino acid (C-terminal amino acid), solid-phase peptide synthesis does not, because a solid support (e.g. resin) acts as the protecting group for the only C-terminal amino acid that requires protection.

Amino acid side chains represent a broad range of functional groups and are therefore a site of considerable side chain reactivity during peptide synthesis. Because of this, many different protecting groups are required, although they are usually based on the benzyl (Bzl) or tert-butyl (tBu) group. The specific protecting groups that can be used during the synthesis of a given peptide vary depending on the peptide sequence and the type of N-terminal protection used. Side chain protecting groups are known as permanent protecting groups, because they can withstand the multiple cycles of chemical treatment during the synthesis phase and are only removed during treatment with strong acids after the synthesis is complete.

In accordance with a further preferred embodiment of the first aspect of the invention at least some of the linear dithiol peptides comprise a primary or secondary amine and the method further comprises modifying the primary or secondary amine with a carboxylic acid, wherein the cyclic peptides comprising a primary or secondary amine and the carboxylic acids are preferably transferred by acoustic dispensing.

By modifying the primary or secondary amine with a carboxylic acid the complexity of a peptide library can be further increase; i.e. the number of different peptides in the library can be significantly increased. This in turn increases the chances to identify an ideal inhibitor or binding molecule in case the peptide library is screened for a binder to or inhibitor of a particular target molecule. This will be explained in more detail in connection with the second aspect of the invention herein below.

Acoustic dispensing is a liquid transfer technique which allows to carry out reactions in very little volume and as illustrated by Example 3 in a volume of only 80 nanoliters. Acoustic dispensing allows non-contact, high-precision, high-speed capability liquid handling by transferring liquids using acoustic ultrasound energy.

The modification of the primary or secondary amine with a carboxylic acid, wherein the cyclic peptides comprise a primary or secondary amine and the carboxylic acids is also the subject of the second aspect of the invention as described herein below. The preferred embodiments and definitions of the second aspect of the invention apply mutatis mutandis to the above further preferred embodiment of the first aspect of the invention.

In accordance with a yet further preferred embodiment of the first aspect of the invention the method further comprises (c) contacting the peptide library preferably without prior purification of the peptide library with a target molecule, and (d) screening the peptide library for a peptide binding to and preferably inhibiting the target molecule.

Means and methods for screening a peptide library for a peptide binding to and modulating the target molecule, and preferably inhibiting the target molecule are known in the art. In a preferred embodiment, the target is an amino acid-based target such as a protein. Generally, the target can also be a non-amino-acid based compound, e.g. an oligo- or polynucleotide.

In this connection the term “inhibiting the target molecule” means that the biological activity is inhibited and preferably completely abolished. Biological activity is the capacity of a specific molecular entity to achieve a defined biological effect on a target. It is measured in terms of potency or the concentration of the molecular entity needed to produce the effect. A biological activity is determined by means of a biological assay.

In accordance with a more preferred embodiment of the first aspect of the invention steps (c) and (d) are carried out in the same wells in which the primary or secondary amine has been modified with a carboxylic acid.

This approach saves material and time. The wells can, for example, be in the format of a multi-well plate, such as 96-well plate, 384-well plate or 1536-well plate.

In accordance with a further more preferred embodiment of the first aspect of the invention the target molecule is a protein or nucleic acid molecule, and is preferably a protein.

Among these options the target is preferably an amino acid-based target such as a protein.

In accordance with another preferred embodiment of the first aspect of the invention the solid phase comprises a resin, preferably an apolar resin and more preferably a polystyrene resin.

Several resins for peptide synthesis are known and commercially available. Non-limiting examples are PEGA resins that consist of 2-acrylamidoprop-1-yl-(2-aminoprop-1-yl) polyethylene glycol 800, resins of poly-ε-lysine cross-linked with sebacic acid, resins of cross-linked hydroxyethylpolystyrene and polyethylene glycol, polyethylene glycol-based resins. Any of the above mentioned resin matrices can be functionalized with reactive groups, including but not limited to aminomethyl-, thiomethyl-, thioethyl-, chloroalkyl-, trityl chloride, HMBA, and Rink amide. A polystyrene resin is preferred since it is used in the examples of the application.

In accordance with another preferred embodiment of the first aspect of the invention the linear dithiol peptides comprise linear dithiol peptides having a molecular weight of less than 1000 Da and preferably of less than 600 Da, and/or linear dithiol peptides comprising 3 or 4 amino acids or building blocks and preferably 3 amino acids or building blocks.

The dithiol peptides of this preferred embodiment are particularly suitable for the “reductive release” according to item (i) of the claimed method. This is because in such peptides the two thiol groups in the dithiol peptides are close to each other, so that they cannot be effectively released by a cyclative release due to conformational constraints.

In the cyclative release strategy as shown in FIG. 1a, peptides are synthesized on solid phase via a disulfide linker and are released via a cyclative disulfide exchange reaction. A major limitation of cyclative release is that particularly short dithiol peptides cannot efficiently be produced due to the path via conformationally constrained disulfide-cyclized peptides. Peptides composed of one amino acid and a thiol-containing building block on each side (three building blocks/amino acids in total), as shown in

FIG. 1a, are not efficiently released. Even some of the peptides containing two amino acids between the thiol-containing building blocks (4 building blocks/amino acids in total) were not efficiently released if the peptides were restrained in their conformational flexibility (e.g. through amino acids having constraints for Phi and Psi angles). The limitation of the cyclorelease approach to deliver only dithiol peptides longer than three or four building blocks prevents the generation of macrocyclic compounds with molecular weights below around 600 Da, and thus macrocyclic compounds that would be particularly attractive for developing oral or cell permeable drugs.

The dithiol peptides to be used for the “cyclative release” according to item (ii) of the claimed method therefore preferably have a molecular weight of above 600 Da, and/or comprise more than 3 amino acids, more preferably more than amino acids.

The present invention relates in a second aspect to a method for the diversification of a macrocyclic compound library, preferably a cyclic peptide library, wherein at least some macrocyclic compounds, preferably cyclic peptides comprise a primary or secondary amine, wherein the method comprises modifying the primary or secondary amine with one or more carboxylic acids.

The definitions and preferred embodiments of the first aspect of the invention as far as being amendable to the second aspect of the invention apply mutatis mutandis to the second aspect of the invention.

The term “macrocyclic molecule” refers to a molecule, wherein a series of 12 or more atoms is connected to form a macrocyclic ring or cycle. The macrocyclic molecule is preferably a cyclic peptide as defined in connection with the first aspect of the invention but the macrocyclic molecule is not necessarily based on a peptides and a macrocyclic molecule may also have a structure containing peptide and non-peptide components. Examples of non-peptide components include hydrocarbons, ethers, esters, amides, aryls, sugars, ketones, epoxides and amines. Examples of non-peptide macrocycles include rapamycin, macrolide antibiotics, lorlatinib and simeprevir.

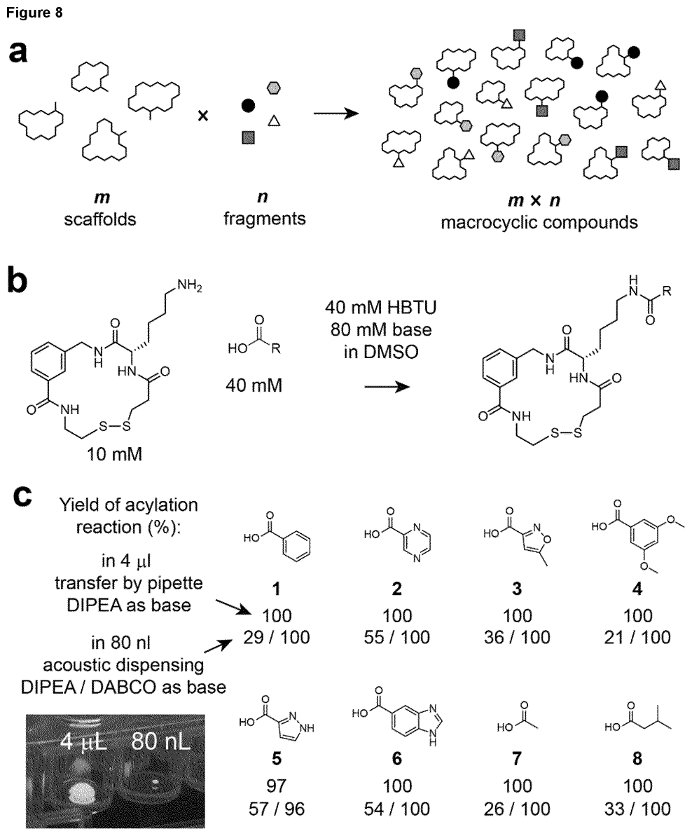

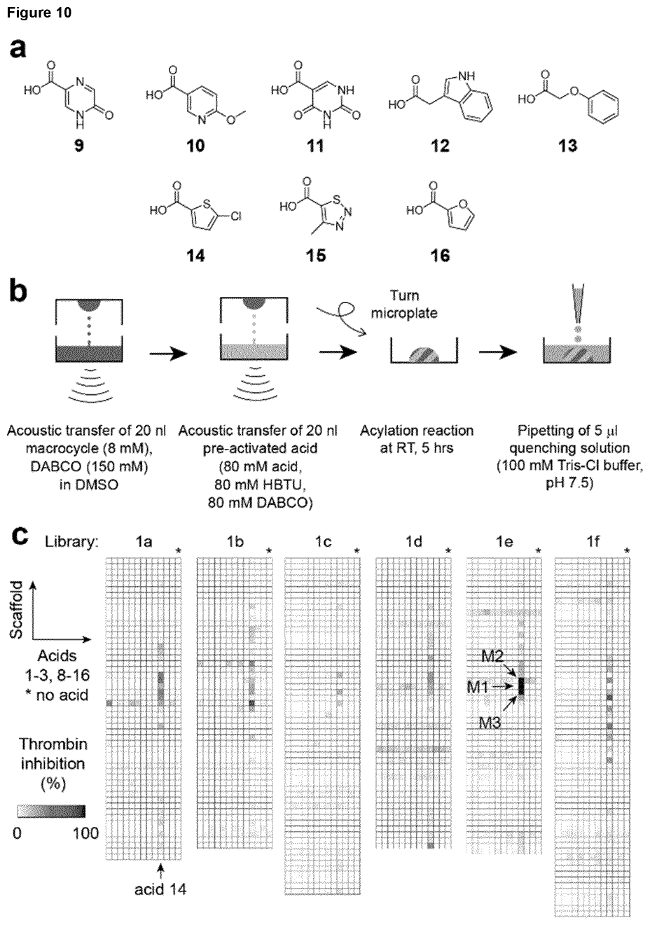

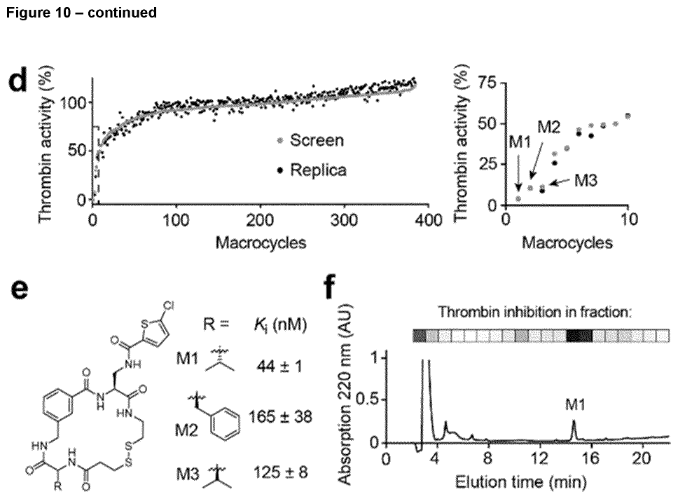

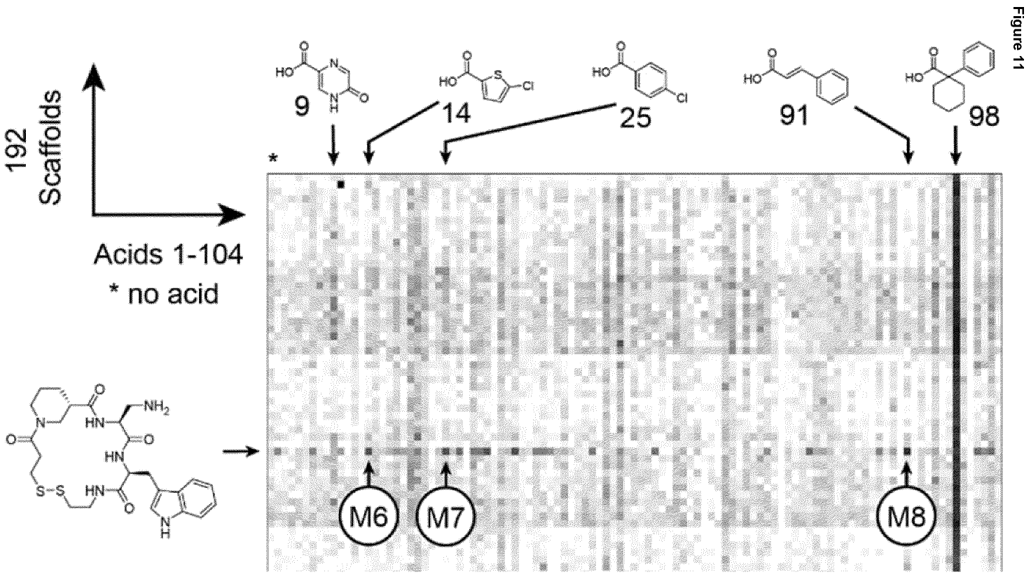

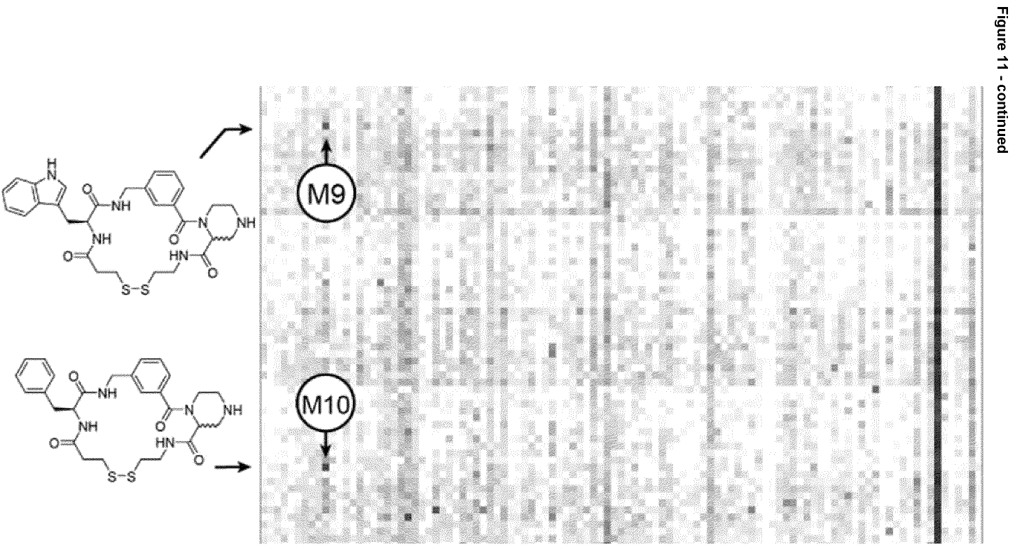

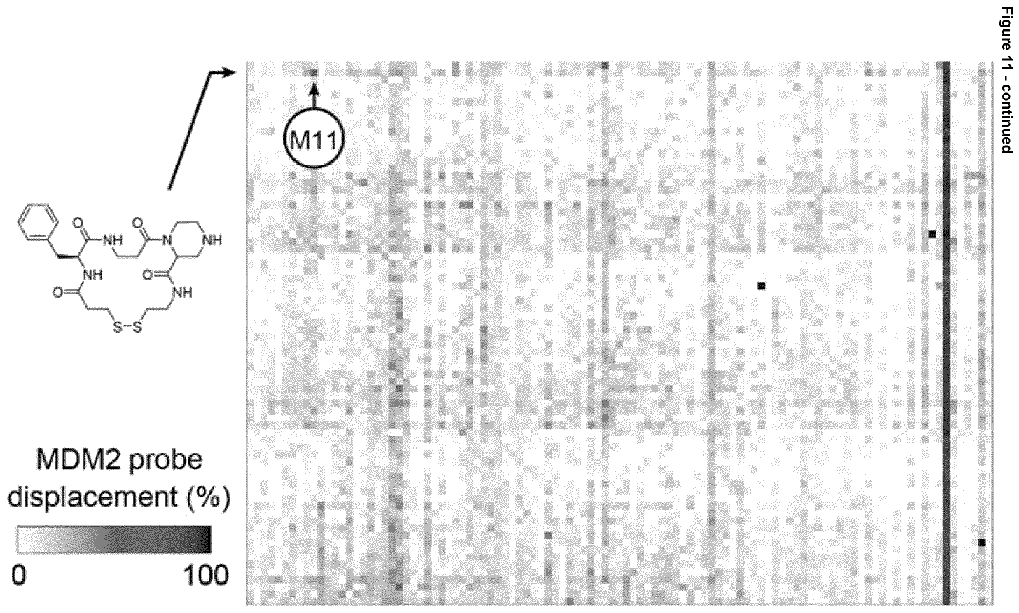

The nature of the carboxylic acids to be used is not particularly limited and preferred examples are shown in FIGS. 8c and 10a. The complexity of the library can be adjusted by the number of different carboxylic acids to be used.

The cyclic peptides or macrocyclic compounds to be modified are preferably contacted with a 2-fold to 20-fold, preferably 3-fold to 15-fold and most preferably 4-fold to 12-fold excess of carboxylic acids, since this excess increases the reaction efficiency.

The carboxylic acids are preferably activated carboxylic acids. An activated carboxylic acid is a derivative of a carboxyl group that is more susceptible to nucleophilic attack than a free carboxyl group, for example, acid anhydrides, acyl chlorides, thioesters and esters.

In order to activate carboxylic acids an acid-activation agent may be used. It follows that the method of the second aspect of the invention preferably comprises an acid-activation agent being capable of activating the one or more carboxylic acids

The acid-activation agent is preferably HBTU ((2-(1H-benzotriazol-1-yl)-1,1,3,3-tetramethyluronium hexafluorophosphate; Hexafluorophosphate Benzotriazole Tetramethyl Uronium) as used in the examples. Other suitable acid-activation agents that may be employed are HATU ((1-[bis(dimethylamino)methylene]-1H-1,2,3-triazolo[4,5-b]pyridinium 3-oxide hexafluorophosphate), HSTU (N,N,N′,N′-tetramethyl-O-(N-succinimidyl)uronium hexafluorophosphate), TSTU (N,N,N′,N′-Tetramethyl-O-(N-succinimidyl)uronium tetrafluoroborate), TPTU (O-(2-Oxo-1(2H)pyridyl)-N,N,N′,N′-tetramethyluronium tetrafluoroborate), and DMTMM BF₄ (4-(4,6-Dimethoxy-1,3,5-triazin-2-yl)-4-methylmorpholinium tetrafluoroborate).

The method of the second aspect of the invention preferably comprises a base. The base assists in activating and coupling of the carboxylic acid. Preferred examples of thereof will described herein below.

The cyclic peptides or macrocyclic compounds preferably comprise a primary or secondary amine as a peripheral group (as illustrated in FIG. 8) or a secondary amine within the backbone. In the case of cyclic peptides, the amino groups can be introduced through side chains of amino acids or through the N-terminal amino acids.

The generation of macrocycle-based ligands is currently hindered by the lack of large libraries of macrocycles, noting that the chances of isolating a macrocycle-based ligand binding with high affinity to a selected target from the library increase with the number of different potential binding partners in the library. The limitation of obtaining large libraries of macrocycles is overcome by the method of the second aspect of the invention. The approach of this method is based on tethering chemically diverse fragments to peripheral groups of structurally diverse macrocyclic scaffolds in a combinatorial fashion. In a proof-of-concept, the generation of a library of 19,968 macrocycles by conjugating >104 carboxylic acid fragments to 192 macrocyclic scaffolds is illustrated in Example 3. The high reaction efficiency and small number of side products of the acylation reactions allowed for high-throughput screening (HTS) of the library without prior purification. Example 3 also illustrates the successful isolation of a low nanomolar thrombin inhibitor (Kᵢ=44 nM) and a high nanomolar MDM2/p53 protein-protein interaction inhibitor (Kᵢ=390 nM) from the library.

The approach of the second aspect of the invention offers a dramatic increase in the rate at which macrocycles can be synthesized and screened and is generally applicable to any target.

In accordance with a preferred embodiment of the second aspect of the invention, the library is in the format of an article of manufacture as described in connection with the first aspect of the invention.

The preferred embodiments of an article of manufacture as described in connection with the first aspect apply mutatis mutandis to the second aspect. In accordance with this preferred embodiment of the second aspect of the invention, the cyclic peptides or macrocyclic compounds comprising a primary or secondary amine are reacted with the carboxylic acids in the wells of the article of manufacture.

In accordance with a more preferred embodiment of the second aspect of the invention one or more the cyclic peptides, the one or more carboxylic acids, the acid-activation agent, and or the base are transferred to the article of manufacture by acoustic dispending.

Further details on acoustic dispensing have been described herein above in connection with the first aspect. The use of acoustic dispensing is illustrated in Example 3.2 herein below.

Acoustic dispending uses acoustic waves and is preferably an acoustic droplet ejection technology. Acoustic dispensing has the great advantage that reagents in particular in a nanomolar volume can be transferred contact less, which does not require pipetting tips, accelerating the speed of dispensing and reducing waste and costs.

In accordance with a further preferred embodiment of the second aspect of the invention, the macrocyclic compounds, preferably cyclic peptides are screened after the diversification without prior purification.

The screening preferably comprises (a) contacting the macrocyclic compound, preferably cyclic peptide library with a target molecule, and (b) screening the macrocyclic compound, preferably cyclic peptide library for a compound, preferably a peptide binding to and preferably inhibiting the target molecule as described herein above in connection with the first aspect of the invention.

In connection with the second aspect of the invention and in particular the above preferred embodiment without prior purification it is preferred that the cyclic peptide library has been produced by the cyclative release of the first aspect of the invention or that the method of the second aspect comprises prior to the diversification of a cyclic peptide library the steps of producing a cyclic peptide library in accordance with the first aspect of the invention.

Also the cyclic peptide library that has been produced by the cyclative release of the first aspect of the invention can be screened without prior purification. Hence, in case the cyclic peptide library has been produced by the cyclative release of the first aspect of the invention or the method of the second aspect comprises prior to the diversification of a cyclic peptide library the steps of producing cyclic peptide library in accordance with the first aspect of the invention, the cyclic peptide library can be produced and diversified without the need of a purification step prior to the screening.

In accordance with a more preferred embodiment of the second aspect of the invention, the macrocyclic compounds, preferably the cyclic peptides are therefore modified with the one more carboxylic acids and screened in the same wells of the article of manufacture in which they were synthesized.

This may be achieved by dispensing to wells the target molecule (e.g. a protein) and optionally other required screening assay reagents and measuring the binding, for example, by a plate reader. Cyclization in this context is again preferably performed according to the cyclative release of the first aspect of the invention.

In accordance with a further preferred embodiment of the second aspect of the invention, the base is DABCO, the sodium salt of HEPES, or NMM and most preferably DABCO.

While also volatile bases such as DIPEA can be used, volatile bases may evaporate when being dispensed, in particular in 2.5 nl droplets by acoustic waves. The use of non-volatile bases, such as DABCO (1,4-diazabicyclo[2.2.2]octane), or the sodium salt of HEPES (4-(2-hydroxyethyl)-1-piperazineethanesulfonic acid) is therefore preferred herein. Among volatile bases, NMM (N-methylmorpholine) was found to be suited if applied at larger excess. The best results were obtained with DABCO; see Example 3.

Unless otherwise defined, all technical and scientific terms used herein have the same meaning as commonly understood by one of ordinary skill in the art to which this invention belongs. In case of conflict, the patent specification including definitions, will prevail.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The Figures show.

FIG. 1. Strategy for reductive release of peptides synthesized on solid phase via a disulfide bridge. (a) Recently developed “cyclative disulfide release” reaction. Short peptides such as those containing only one amino acid between the two flanking thiol-containing structures (three building blocks) are not efficiently released due to conformational constraints (indicated by dashed lines). (b) Release of a short dithiol peptides by reduction of the disulfide bridge.

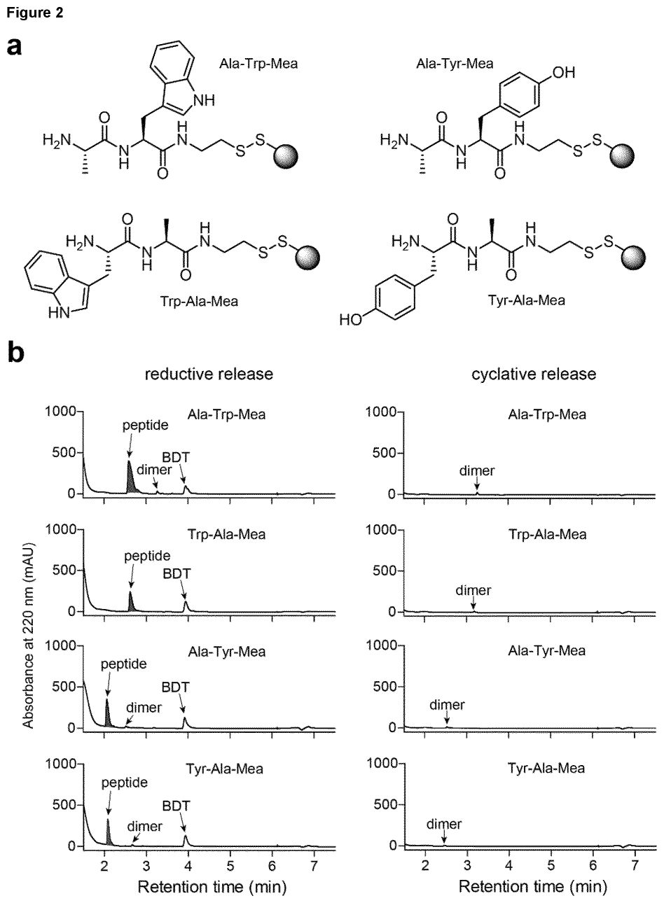

FIG. 2. Reductive release of peptides linked via a disulfide bridge to a solid phase. (a) Chemical structures of four peptides used to test the reductive release of peptides from solid phase. (b) HPLC chromatograms of the four peptides released by reduction of the disulfide-bridge using BDT (100 mM) in DMF containing the base TEA (100 mM TEA) (left). Exposure of the same immobilized peptides to TEA (100 mM) in DMSO, conditions previously used to detach dithiol peptides via cyclative release, led to elution of only small quantities of disulfide-dimerized peptide (right). The peaks of the desired peptides are highlighted in red. The only side product observed is the peptide dimerized via a disulfide bridge (dimer).

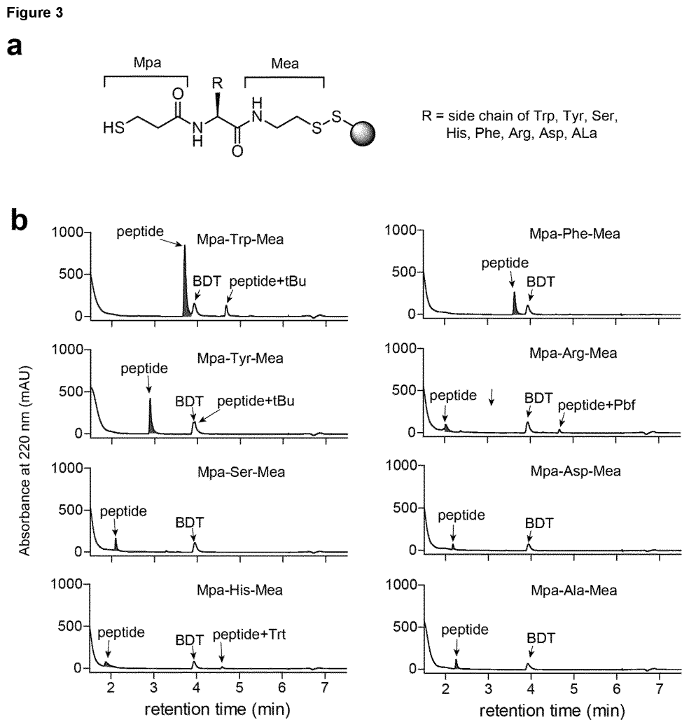

FIG. 3. Reductive release of dithiol peptides linked via a disulfide bridge to a solid phase. (a) Structure of dithiol peptides on solid phase. (b) HPLC chromatograms of eight dithiol peptides released by reduction of the disulfide-bridge using BDT (100 mM) in DMF containing the base TEA (100 mM). The desired peptides are highlighted in red. BDT and the side products are indicated.

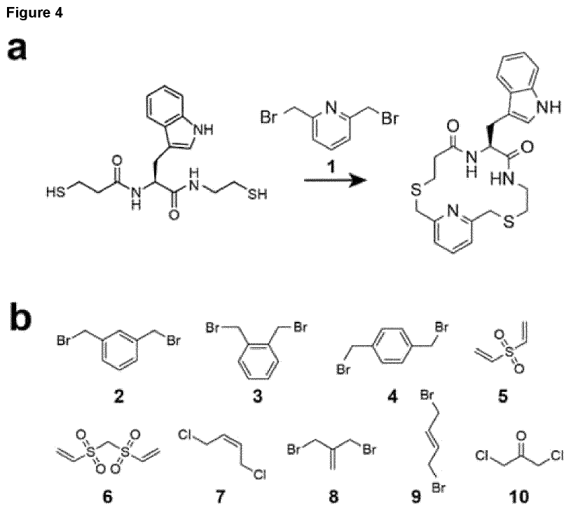

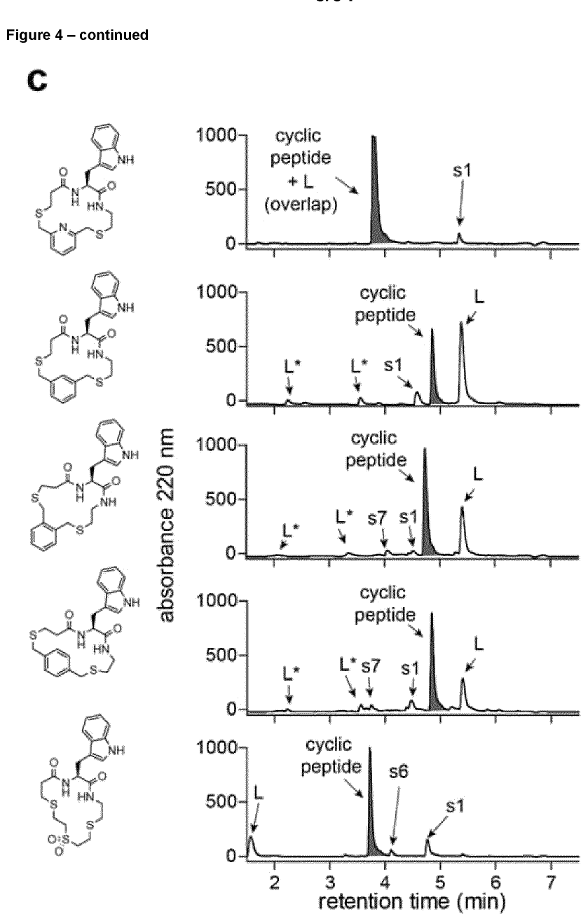

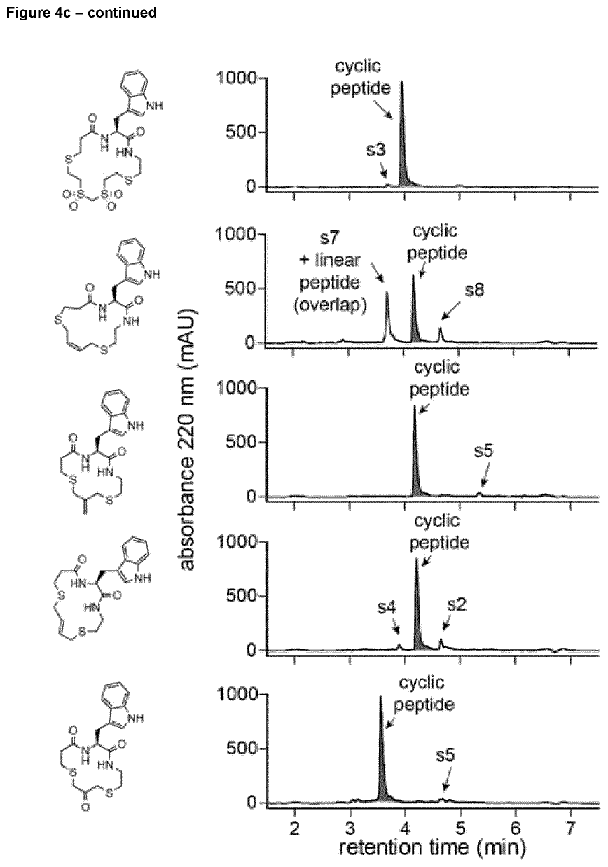

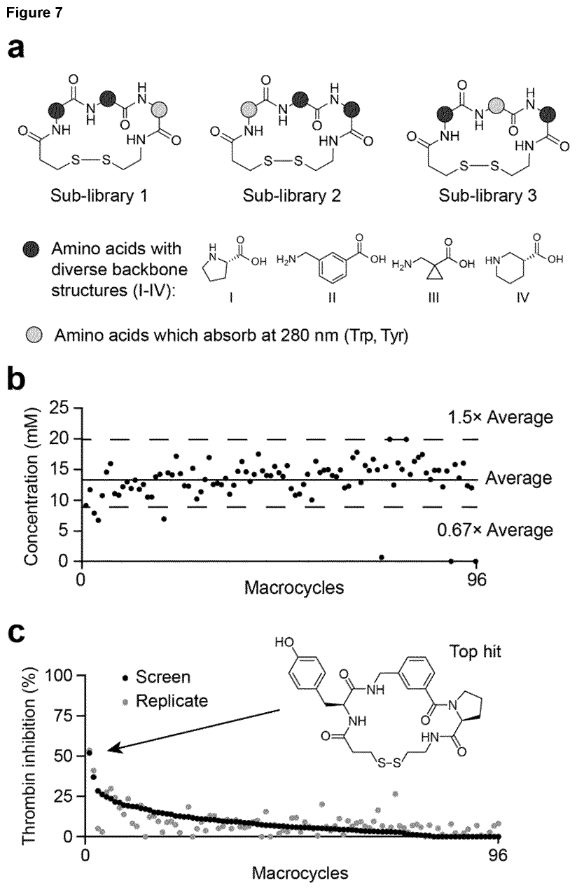

FIG. 4. Cyclization of dithiol peptides by bis-electrophile reagents. (a) Cyclization reaction illustrated with Mpa-Trp-Mea and the reagent 2,6-bis(bromomethyl)pyridine (1). (b) Bis-electrophile reagents 2-10. (c) Chemical structures of macrocycles and HPLC chromatographic analysis of the cyclization reactions. The desired cyclic products are highlighted in red. Side products s1 to s7 are shown in Supplementary FIG. 7. L=bis-electrophile cyclization reagent. L* is hydrolyzed L.

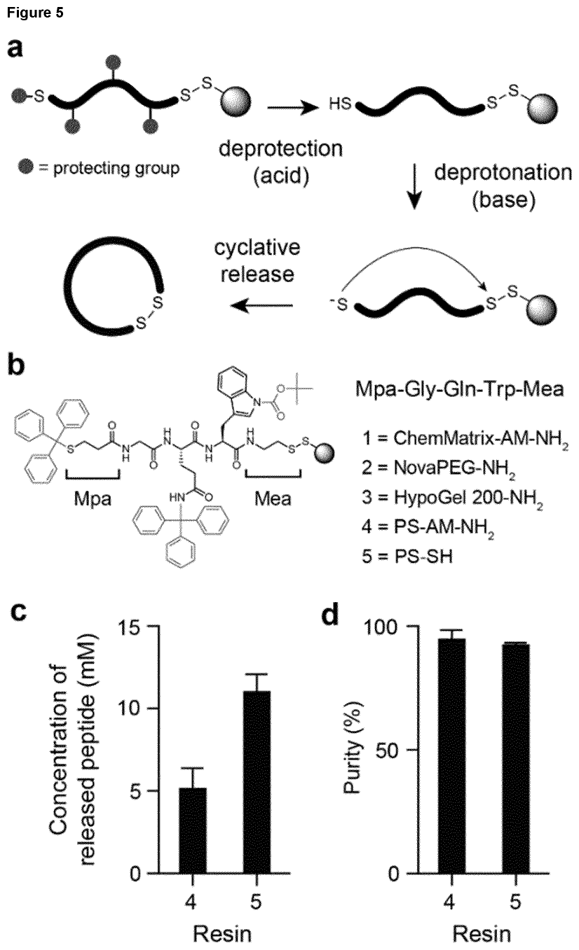

FIG. 5. Cyclative disulfide release strategy. (a) Schematic representation of the strategy. Short peptides are synthesized via a disulfide bridge on solid phase. Removal of protecting groups (red) on solid phase allows for an efficient removal. Treatment with base deprotonates the N-terminal thiol, which induces an intramolecular disulfide exchange to afford the cyclic product. (b) Chemical structure of test peptide Mpa-Gly-GIn-Trp-Mea disulfide-linked to solid support and commercial resins used. (c) Recovery of disulfide-cyclized peptide Mpa-Gly-GIn-Trp-Mea synthesized on resins 4 and 5 and released with 150 mM DIPEA in DMSO. Concentrations were determined by measuring absorbance at 280 nm. Reactions were performed in triplicate. (d) Purity of disulfide-cyclized peptide of panel d. Purity was determined by LCMS, measuring the AUC of all species at 220 nm UV absorbance.

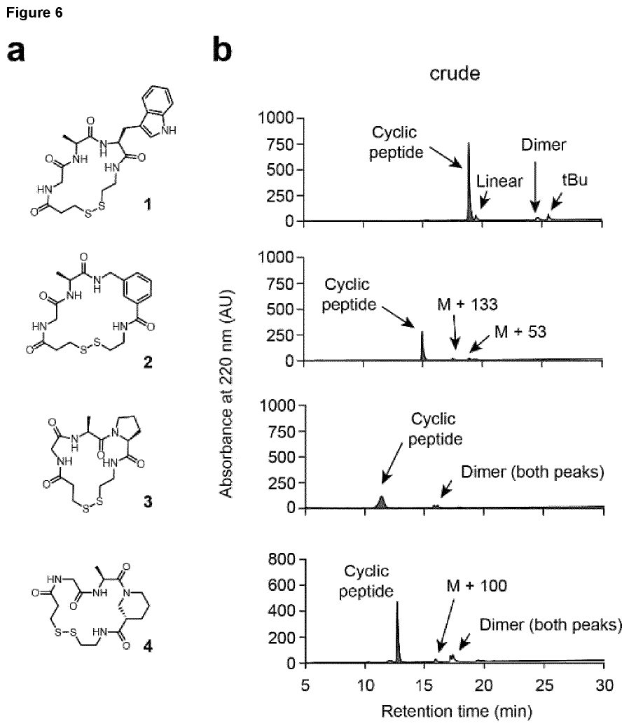

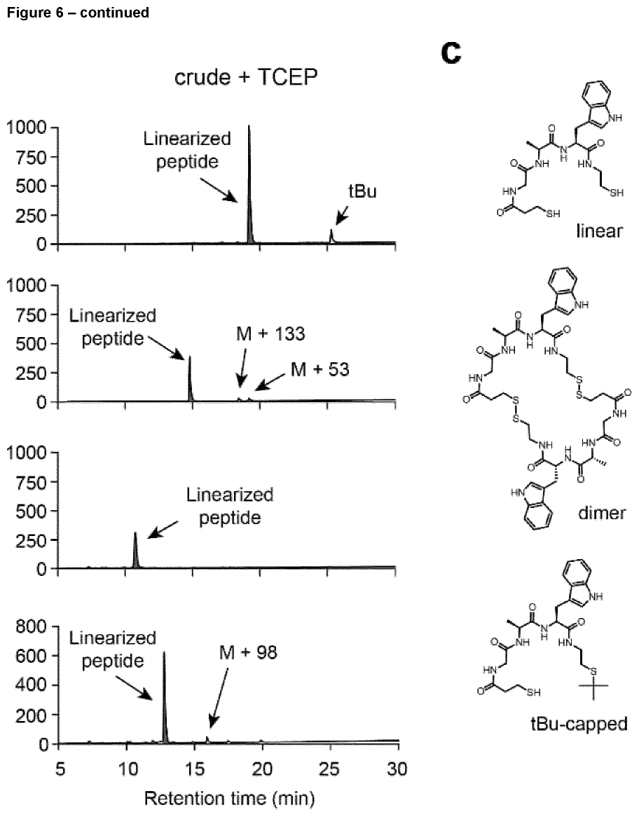

FIG. 6. Cyclative release of peptides with variable sequences. (a) Chemical structures of the desired four cyclic peptides. The peptides are based on the linear sequence Mpa-Gly-Ala-Xaa-Mea with Xaa being an amino acid with variable conformational flexibility in the backbone. (b) Analytical HPLC chromatograms of the crude peptide after cyclative release. The chromatograms on the right show the peptides after disulfide-bond reduction with TCEP. For impurities that were not identified, the mass is indicated (assuming that the species singly charged). (c) Examples of chemical structures that fit with the molecular masses of the identified impurities.

FIG. 7. Library design, peptide recovery quantification by absorption, and thrombin inhibition of the cyclic peptide library. (a) Design of library comprising 96 different peptides and structures of unnatural amino acids used in library. (b) Scatter plot of cyclic peptide concentrations (mM in DMSO) for the 96 synthesized cyclic peptides quantified by absorption at 280 nm. The average recovery, along with 1.5× and 0.67× this value are indicated on the chart. (c) Thrombin inhibition measured at an average cyclic peptide concentration of 11 μM. The peptides are ordered according to their thrombin inhibition activity in the first screen (black dots; highest to lowest activity). Green dots indicate thrombin inhibition for the same cyclic peptides measured in a second screen using the same conditions. The chemical structure of the most active inhibitor is shown (Kᵢ=13±1 μM).

FIG. 8. Diversification of macrocycles by combinatorially appending fragments to peripheral groups. a, General principle of approach. b, Model macrocycle containing a peripheral primary amine is modified by acylation. c, Reaction of model macrocycle with indicated acids. The upper number shows conversion in 4 μL volume by pipetting and the lower number in 80 nl and acoustic liquid transfer, the first number with DIPEA and the second one with DABCO. Images of two droplets in a 96-well plate are shown to demonstrate the difference in scale. The droplets contain fluorescein for and are exposed to UV light for visualization.

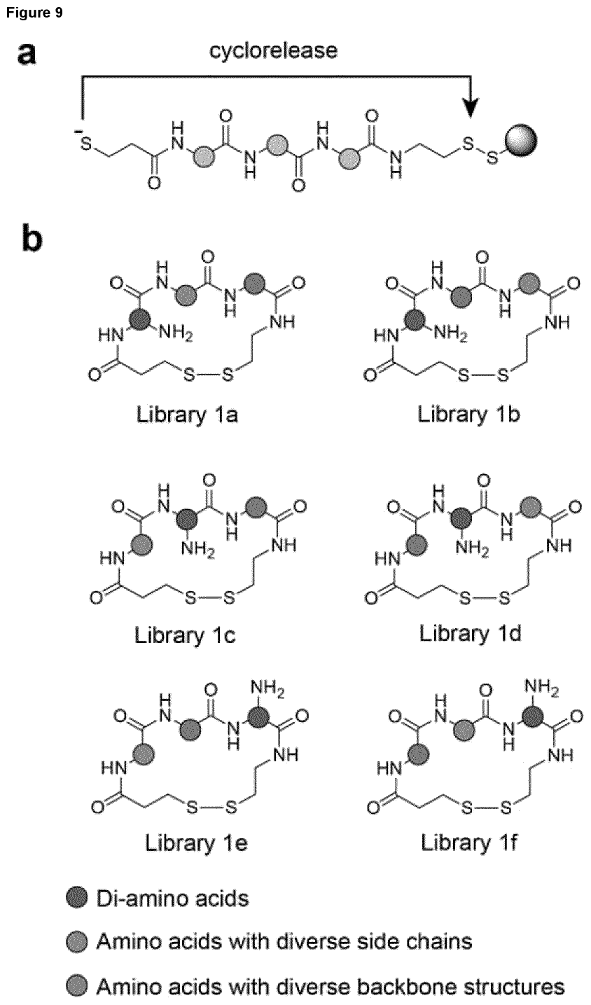

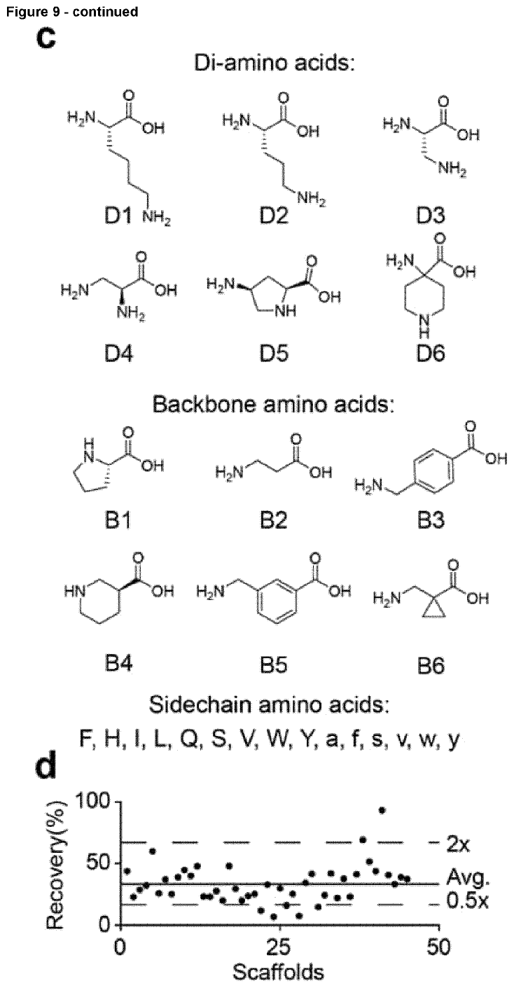

FIG. 9. Preparation of macrocycles containing a peripheral amino group. a, Cyclative disulfide release of side chain-deprotected peptides. b, Format of scaffolds in Library 1. c, Amino acids used for the scaffold library synthesis. d, Yields of 45 tryptophan-containing scaffolds determined by absorption measurement.

FIG. 10. Screening of macrocyclic compound library against thrombin and hit identification. a, Carboxylic acids 9 to 16 that were use along with acids 1 to 3 to diversify scaffold Library 1a-f (shown in FIG. 2). b, Schematic procedure for macrocycle library synthesis by acoustic liquid transfer. Reaction conditions are indicated. c, Heat map showing thrombin inhibition for each macrocycle. The amino acid composition of all macrocycles are shown in Supplementary Table 1. d, Replica reaction and screen of all compounds containing acid 14. e, Chemical structures and activities of top three hits M1 to M3. Mean values and SDs of three independent measurements are shown. f, Chromatographic separation acylation reaction yielding M1 and analysis of fractions for thrombin inhibiting species.

FIG. 11. Screen against Thrombin. 384 macrocycles were synthesized according to our previously described procedure (utilized Fmoc amino acids and their corresponding one-letter codes are shown in Supplementary FIG. 1b). Each macrocycle was reacted with 12 carboxylic acids. Following the reaction and quench, thrombin and fluorogenic substrate were added to the wells, and the increase in fluorescence was measured over 30 minutes. Final macrocycle concentration was 10 μM. Residual thrombin activity was determined by dividing the slope of fluorescence intensity over time for each well by the slope of control wells without macrocycle.

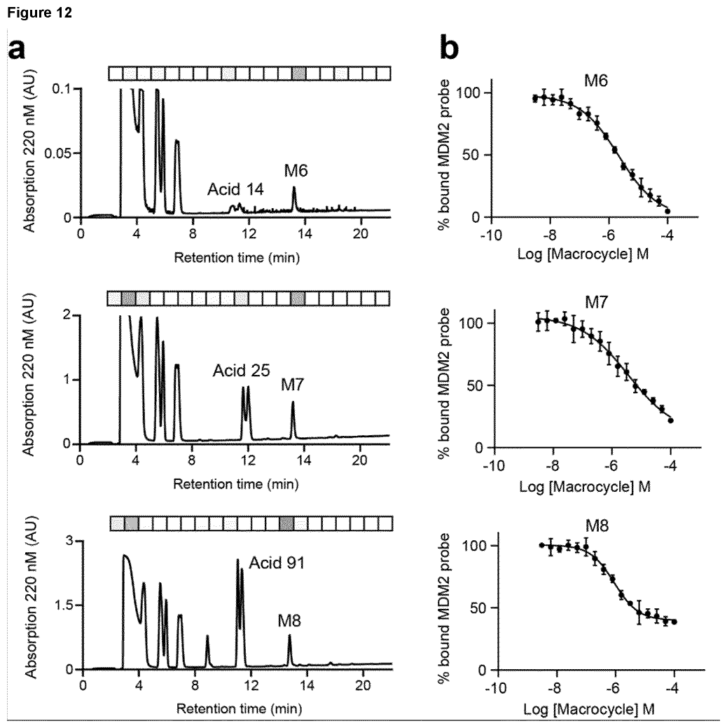

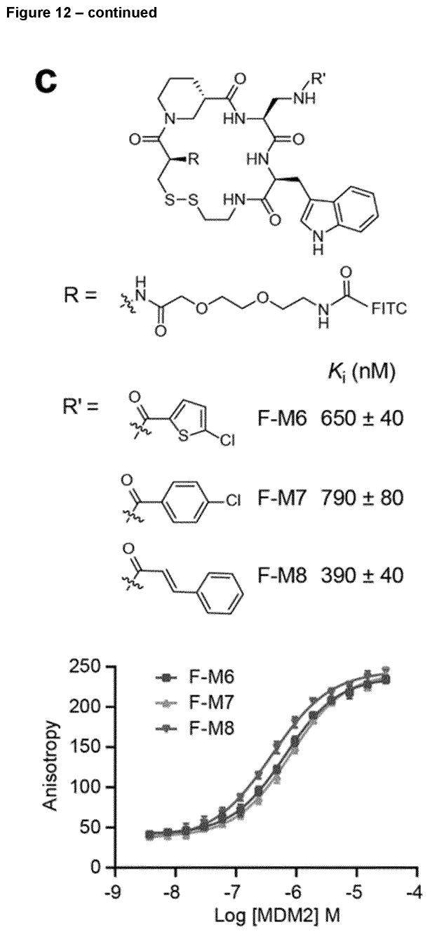

FIG. 12. MDM2 binders. a, Chromatographic separation acylation reaction yielding M6 to M8 and analysis of fractions for thrombin inhibiting species. b, Binding of purified macrocycles to MDM2 measured by testing the displacement of fluorescent MDM2 probe (linear peptide) using fluorescence polarization. Mean values and SDs of three independent measurements are shown. c, Chemical structures of macrocycles labeled with fluorescein (F) and binding to MDM2 measured by fluorescence polarization. Mean values and SDs of three independent measurements are shown.

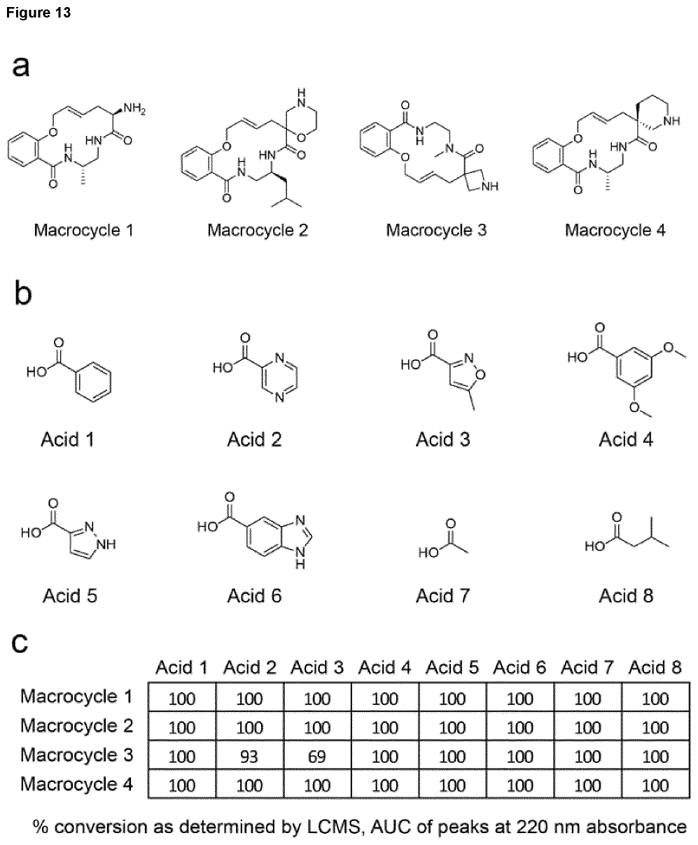

FIG. 13. Diversification of macrocycles by combinatorially appending fragments to peripheral groups. a, Chemical structures of macrocyclic compounds, all containing an amino group. b, Carboxylic acids used to diversify the macrocycles. c, Yields of reactions.

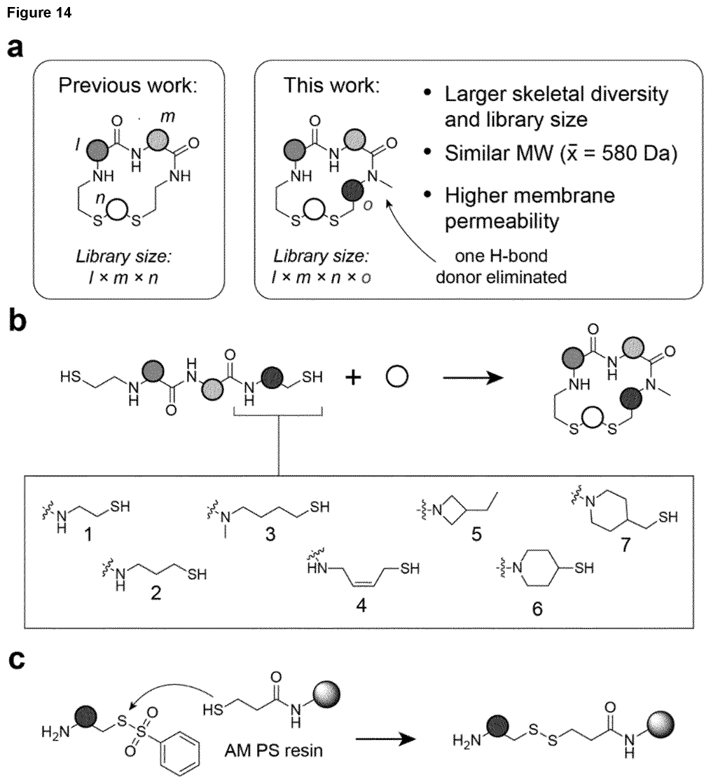

FIG. 14. Strategy for the synthesis of a macrocycle library on the edge of the Ro5. (a) The previous macrocycle strategy and the improved strategy reported in this paper. Circles represent the three groups of building blocks: amino acids (grey), cysteamine and analogs (red), and bis-electrophile linkers (white). (b) Schematic representation of the strategy to cyclize linear peptides by reacting terminal thiol groups by bis-electrophile reagents. Chemical structures of newly developed cysteamine analogs, used as C-terminal building blocks in the linear precursors, are shown. (c) Strategy for synthesizing polystyrene resin carrying cysteamine analogs linked via a disulfide bridge. The cysteamine analogs are loaded to the resin as activated thiosulfonate building blocks.

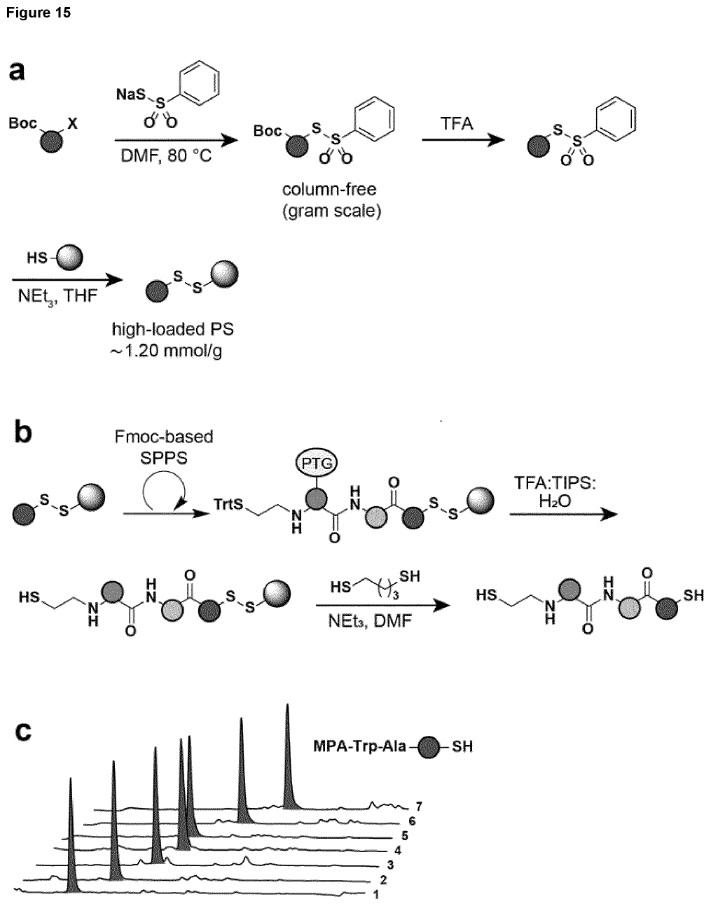

FIG. 15. Synthesis of cysteamine building blocks activated by phenylsulfone and purification without a chromatographic step. (a) Schematic representation of the synthesis of dithiol resins. X=Br or Cl (b) Simplified representation of the purification-free synthesis of dithiol peptides. (c) Stacked HPLC chromatograms (UV220) of the crude peptide quality of a model peptide (MPA-Trp-Ala) synthesized with the 7 different resin building blocks.

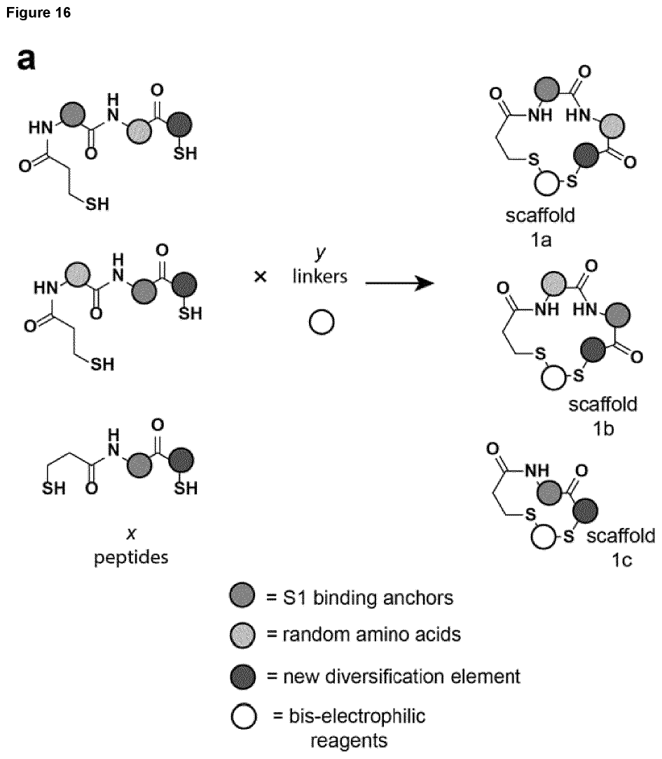

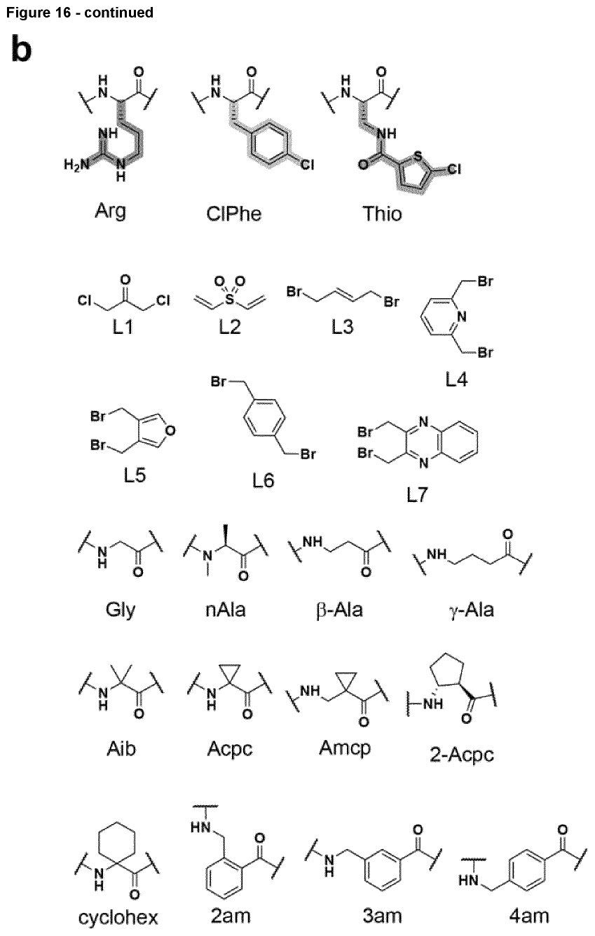

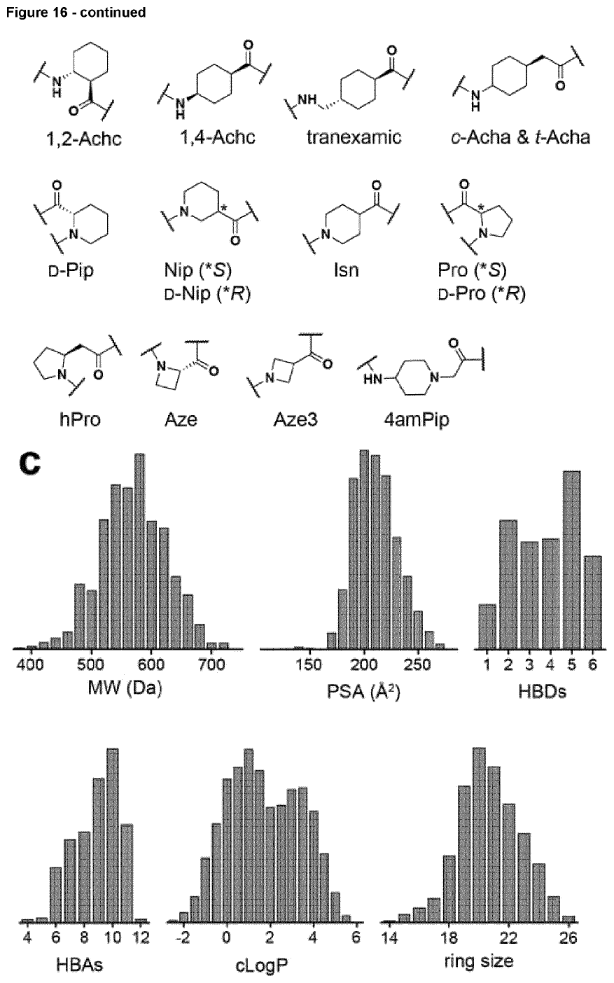

FIG. 16. Design of macrocycle library tailored for generating inhibitors of trypsin-like serine proteases. (a) Format of the designed cyclic peptide library. (b) Amino acids and linkers used for library synthesis. (c) Histograms of selected properties of the generated macrocycle library. Predicted physiochemical properties of the macrocyclic library calculated using DataWarrior (vers. 5.5.0). Marked in green is the area in accordance with ro5 and/or are within the range predicted to allow for cell-permeable macrocycles.

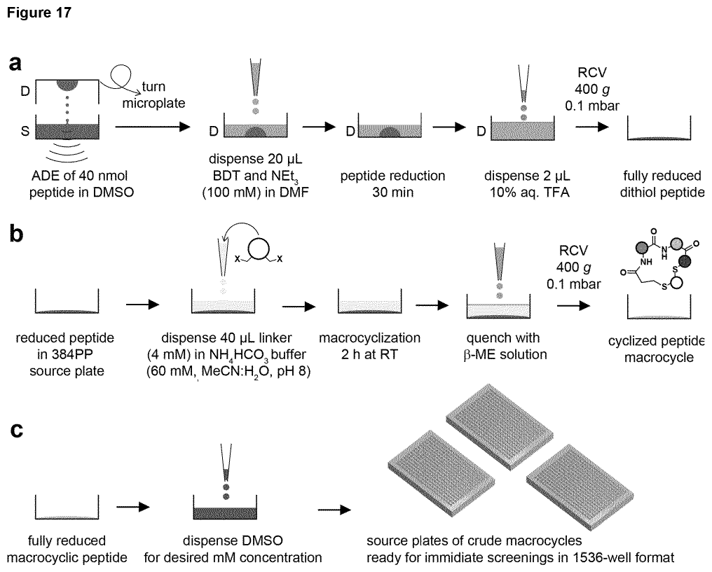

FIG. 17. Experimental steps for library generation. Workflow for the synthesis of macrocycle libraries in microtiter plates.

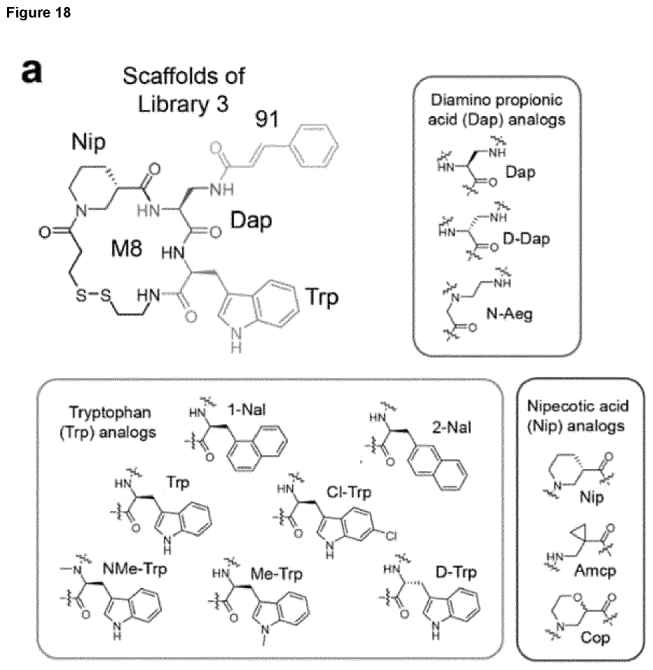

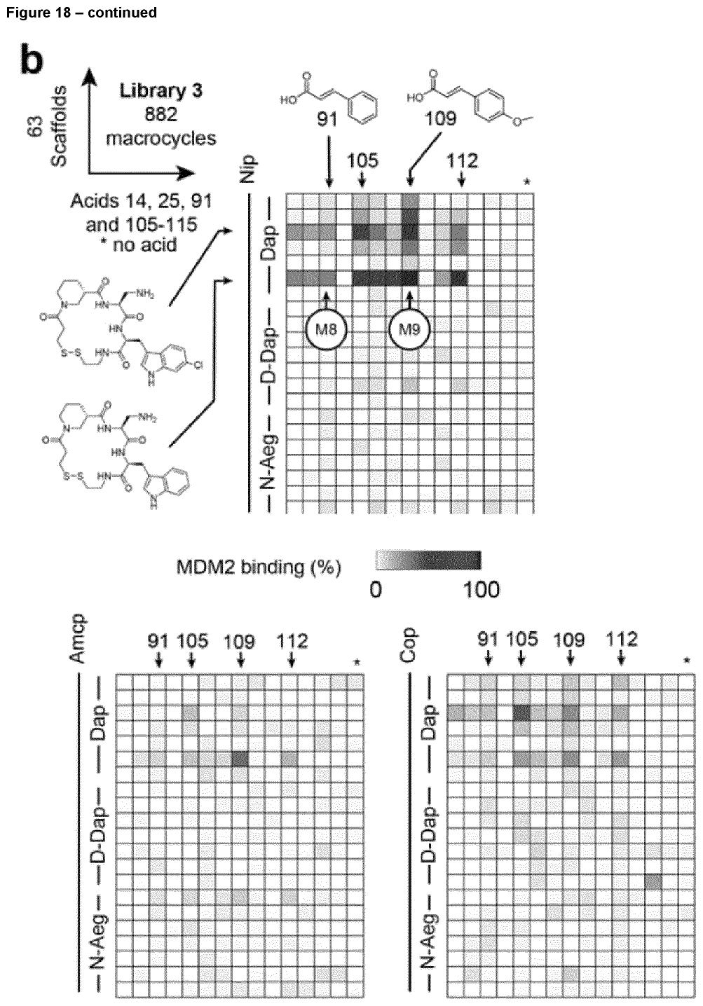

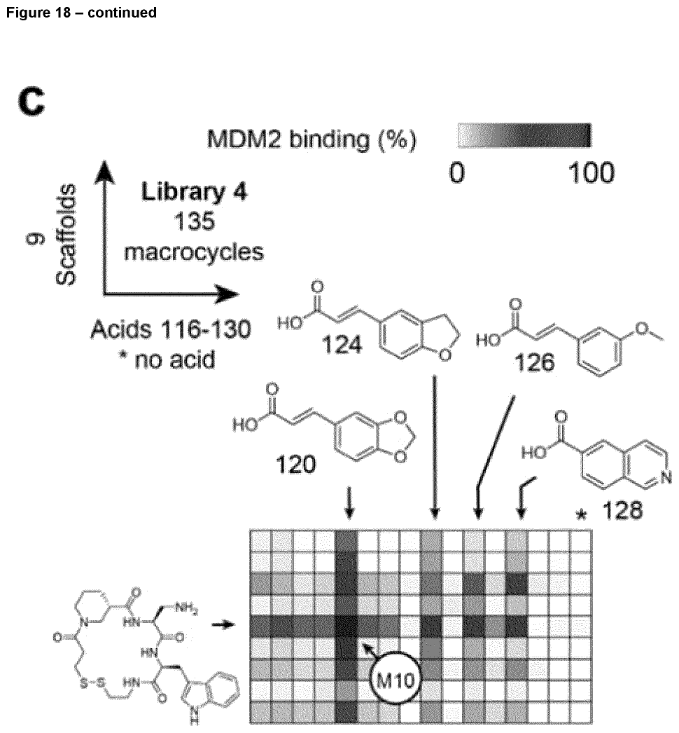

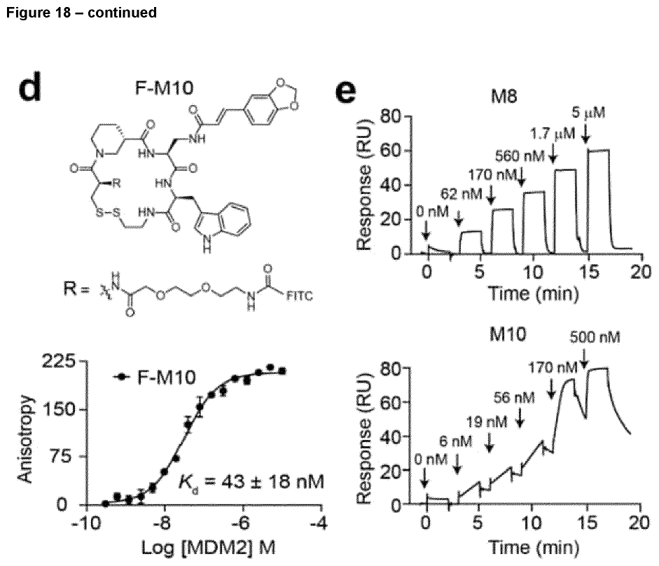

FIG. 18. Affinity optimization of an MDM2:p53 inhibitor. a, Scaffolds of Library 3 are based on M8 wherein the amino acids shown in blue colors are diversified. Amino acid building blocks are shown in the three frames. b, Heatmaps of MDM2 binding to the 63 scaffolds that were combinatorially acylated with 15 carboxylic acids at a 30 pmol scale. Binding to MDM2 was measured by displacement assay of the fluorescent peptide probe by macrocycles at a concentration of 750 nM. c, Screening of Library 4 based on the best nine scaffolds from the previous screens and 15 additional carboxylic acids. d, Binding of fluorescein-labeled and HPLC-purified macrocycle M10 (F-M10) to MDM2 as measured by FP. Mean values and SDs of three independent measurements are shown. e, Binding of unlabeled macrocycles M8 and M10 to MDM2 as measured by SPR.

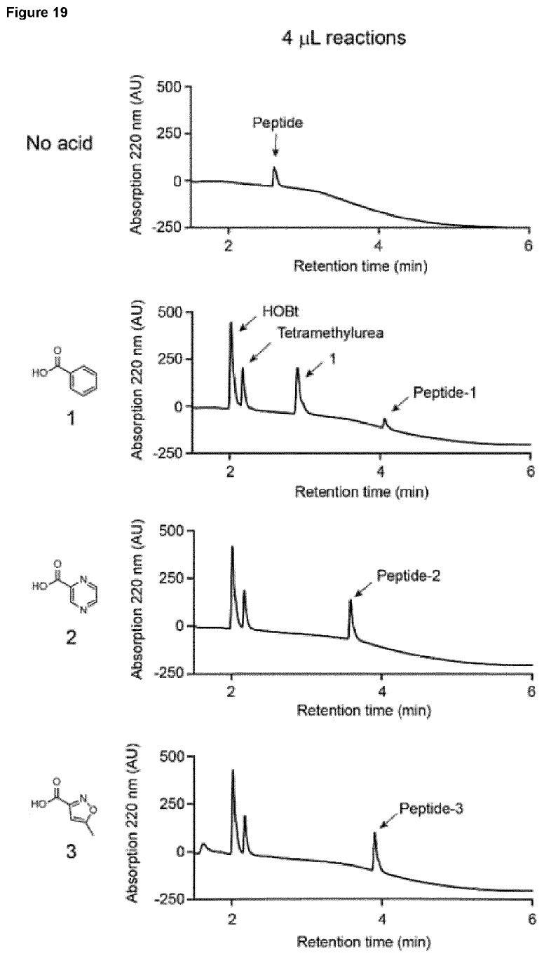

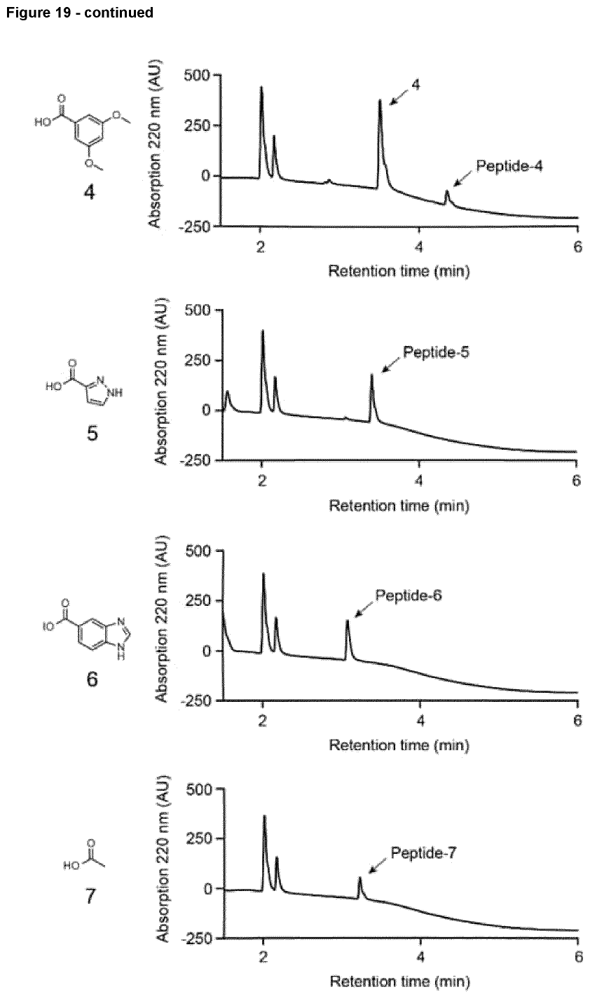

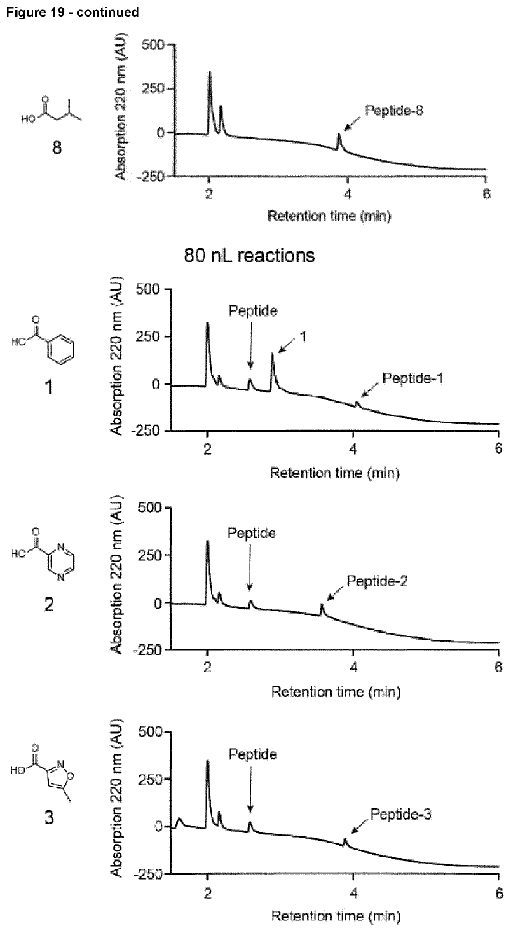

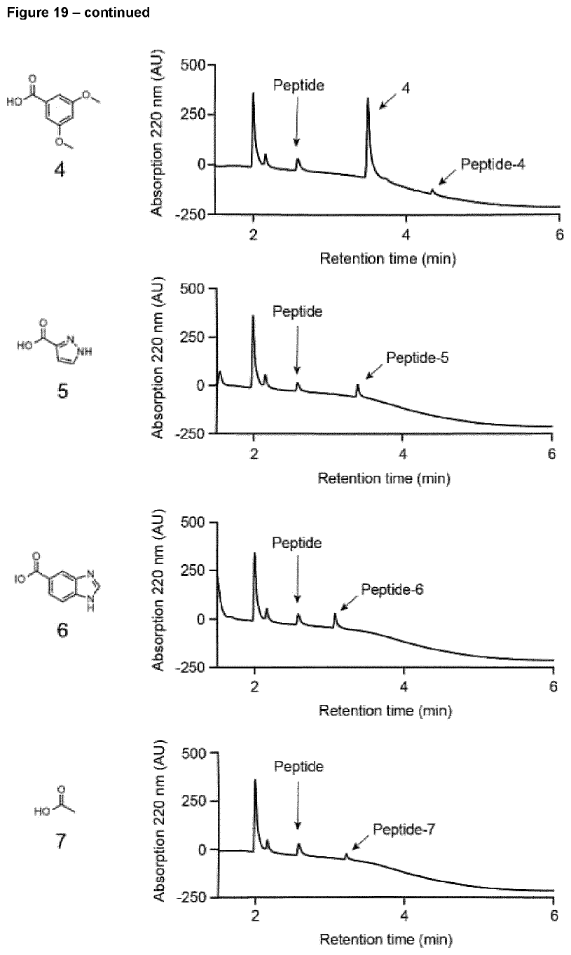

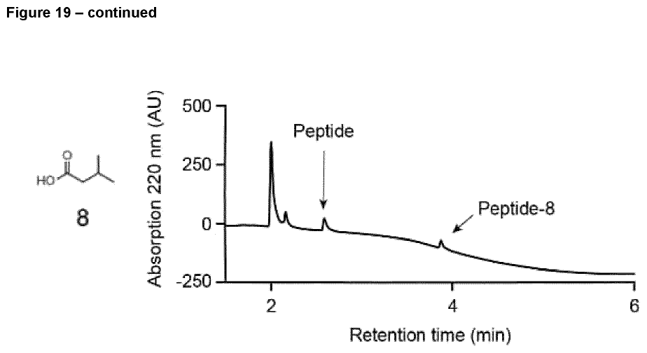

FIG. 19. Comparison of acylation reactions by pipetting (4 μl volume) and acoustic transfer (80 nl volume) using DIPEA as base. The reactions were diluted 100-fold with water and samples of 5 μl analyzed by LC-MS using a RP column and a 0-60% MeCN/H2O gradient over 5 minutes. In the case of the 80 nl reaction volume, two samples were pooled.

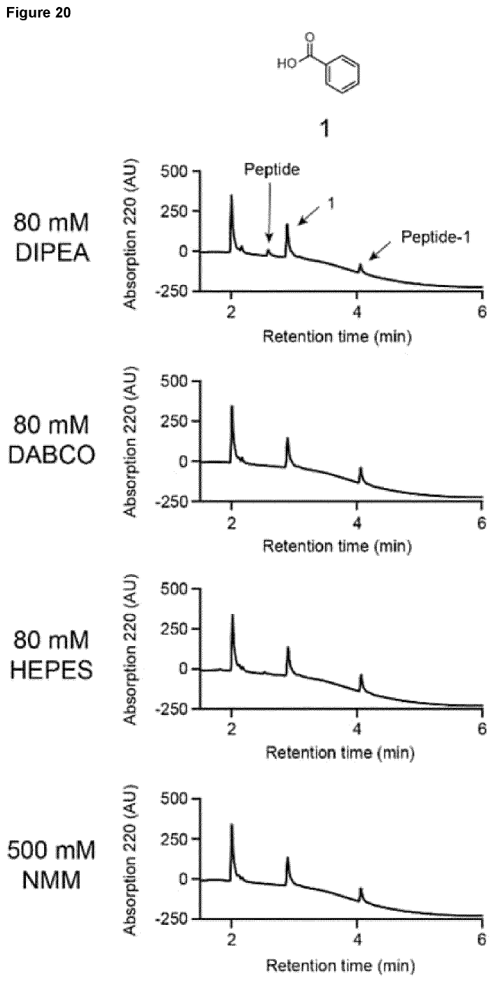

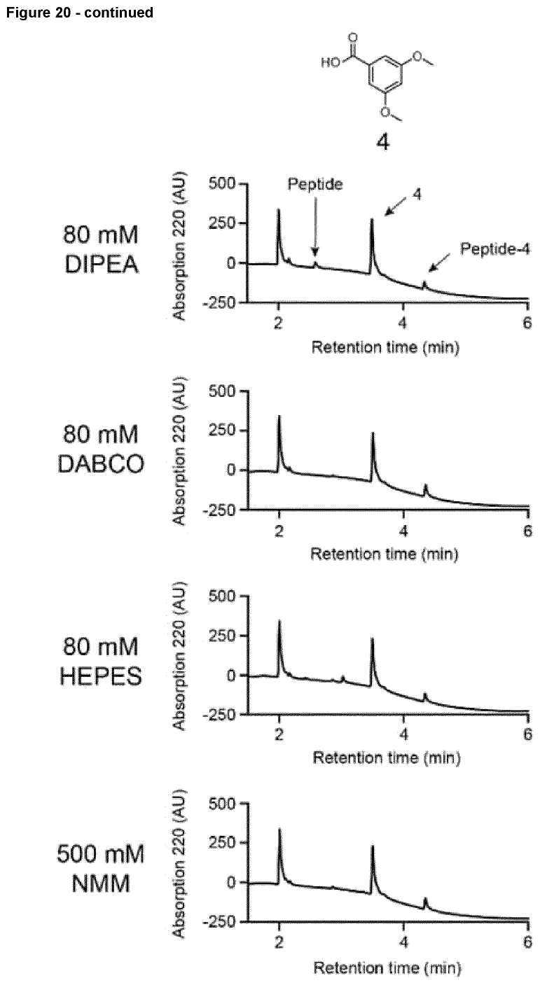

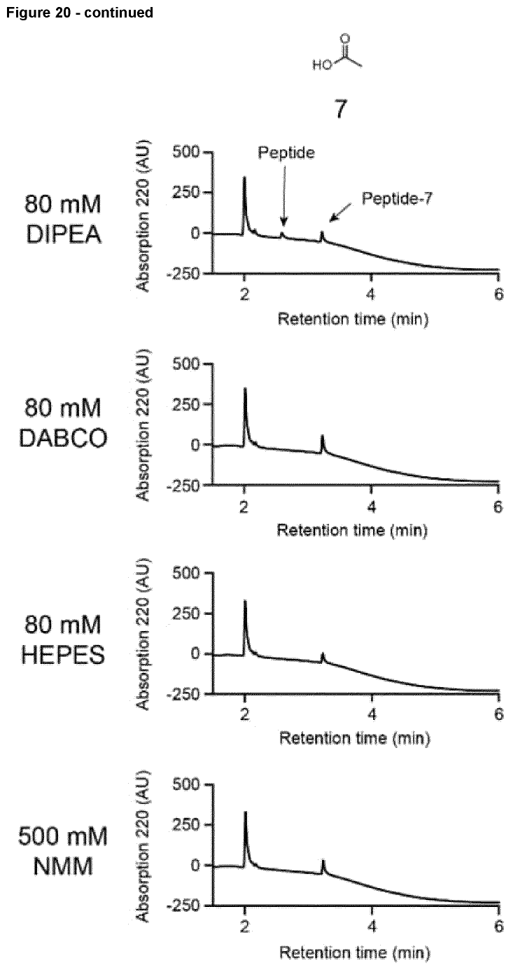

FIG. 20. Acylation reactions in 80 nl volumes using acoustic dispensing and different bases. Two non-volatile bases (DABCO and HEPES sodium salt) at 80 mM concentration, and a volatile base (NMM) at 500 mM concentration were tested as alternatives to 80 mM DIPEA for the acylation of a model scaffold by three carboxylic acids. The reactions were diluted 100-fold with water and samples of 5 μl analyzed (two pooled reactions) by LC-MS using a RP column and a 0-60% MeCN/H2O gradient over 5 minutes. While reactions with DIPEA did not go to completion, all other bases resulted in quantitative conversion to product.

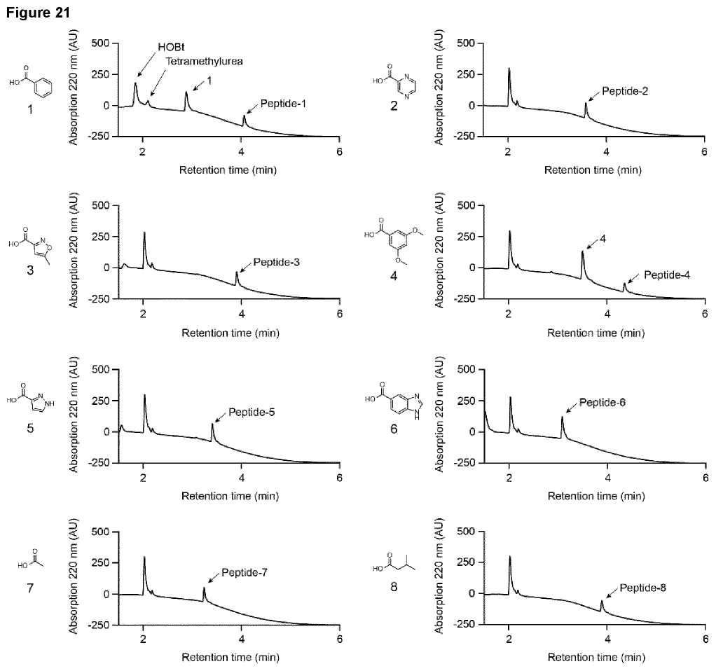

FIG. 21. Comparison of acylation reactions in 80 nL volumes using acoustic dispending and DABCO as base. The model scaffold 1 was reacted with carboxylic acids 1-8 in 80 nl volumes using DABCO as base (80 mM). The reactions were diluted 100-fold with water and samples of 5 μl analyzed (two pooled reactions) were analyzed by LC-MS using a RP column and a 0-60% MeCN/H2O gradient over 5 minutes.

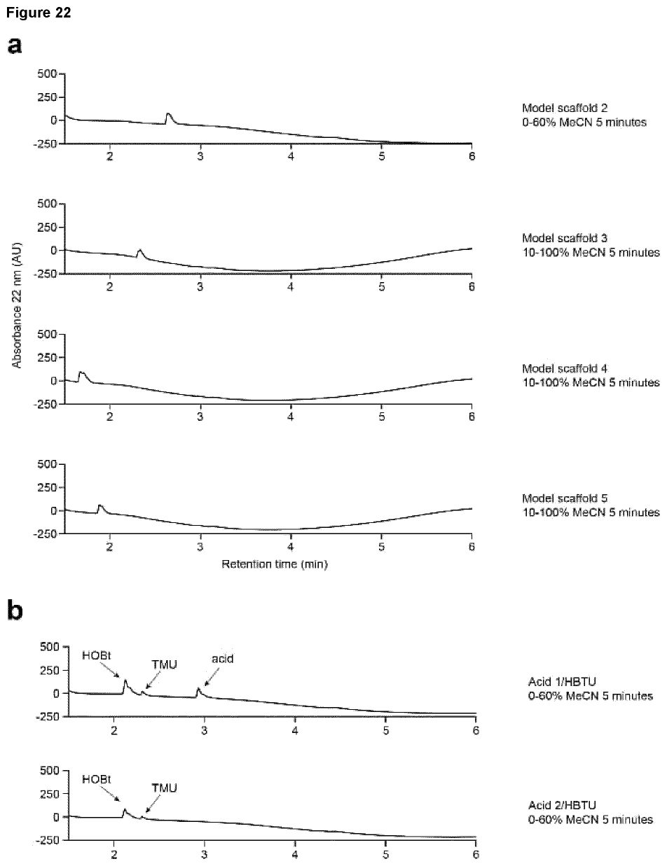

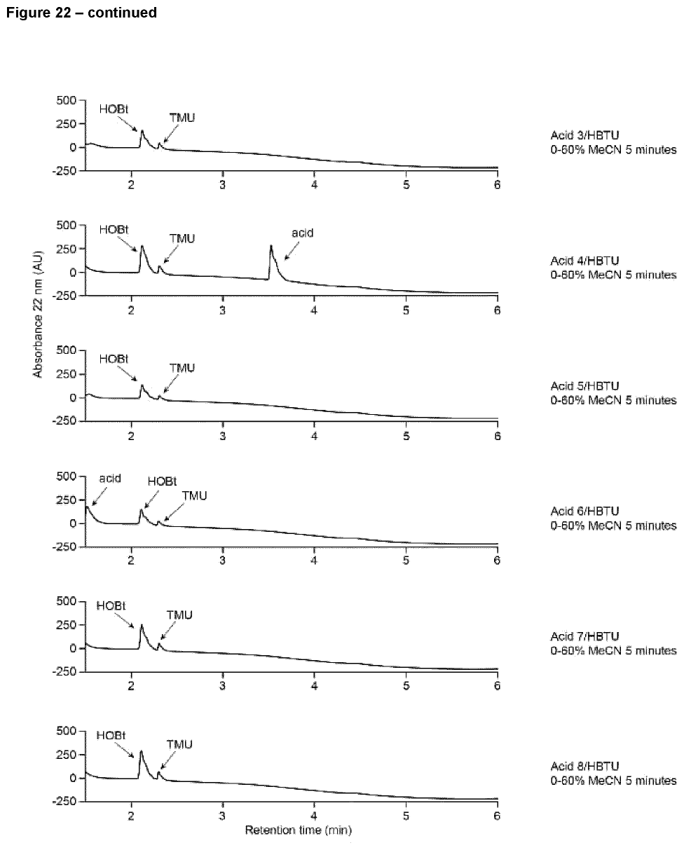

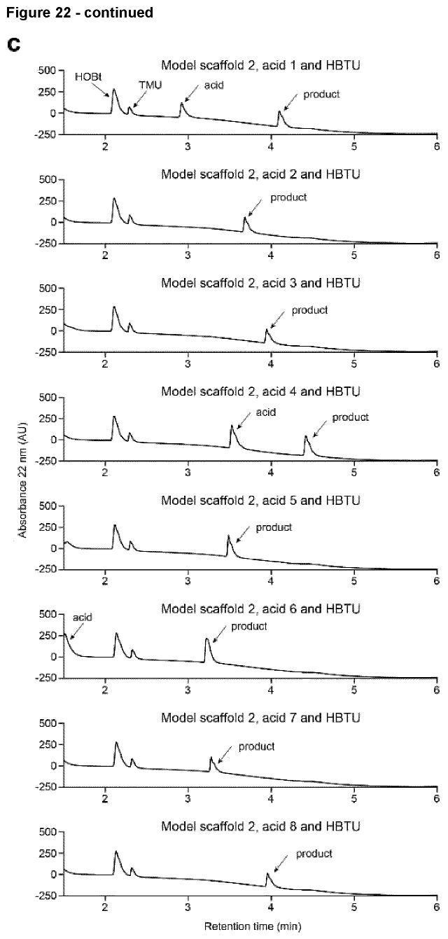

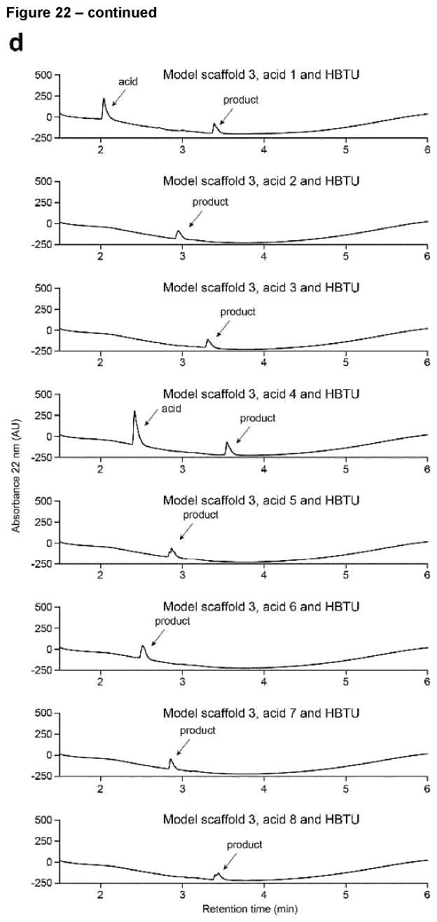

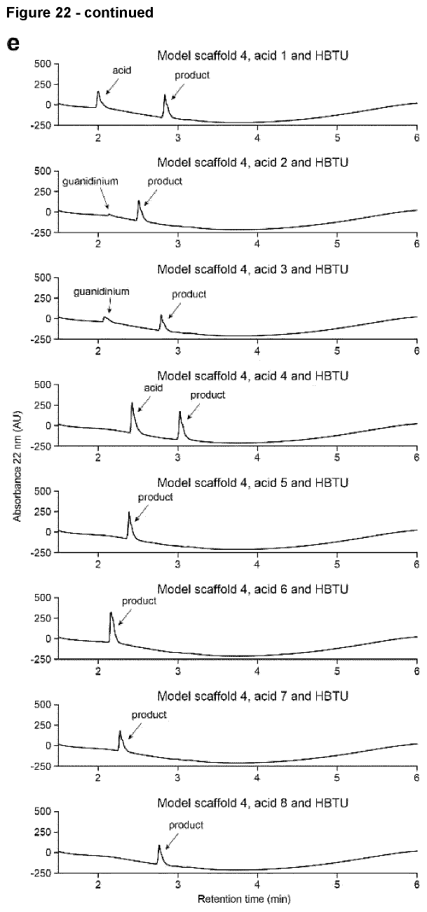

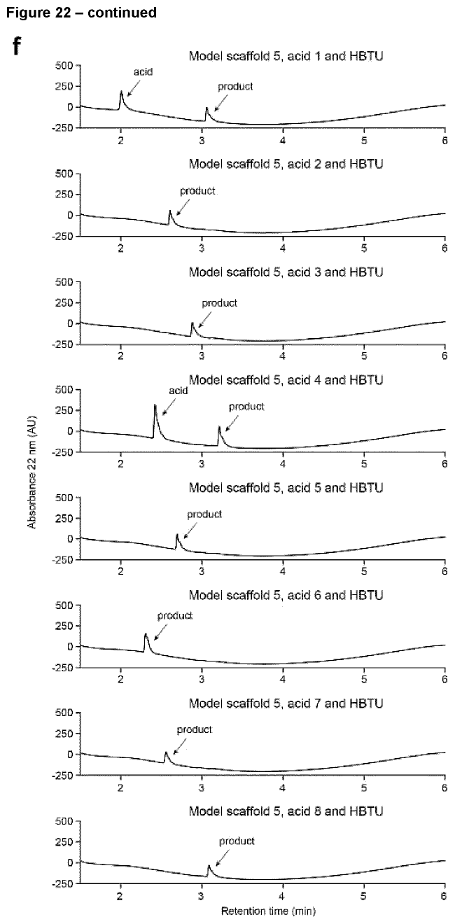

FIG. 22. Acylation of model scaffolds 2 to 5 in 80 nl volumes and acoustic dispensing. a, LC-MS analysis of model scaffolds. The reactions were diluted 100-fold with water and samples of 5 μl were analyzed. Solvent B gradient: 0-60% MeCN, 5 minutes for model scaffold 2; 10-100% MeCN, 5 minutes for model scaffolds 3-5. b, LC-MS analysis of acids. The reactions were diluted 100-fold with water and samples of 5 μl were analyzed. Solvent B gradient: 0-60% MeCN, 5 minutes. TMU=tetramethyl urea. c-f, UHPLC analysis of acylation reactions for model scaffolds 2 (c), 3 (d), 5 (e) and 5 (f). The reactions were diluted 100-fold with water and samples of 5 μL were analyzed. Solvent B gradient: 0-60% MeCN, 5 minutes for model scaffold 2; 10-100% MeCN, 5 minutes for model scaffolds 3-5.

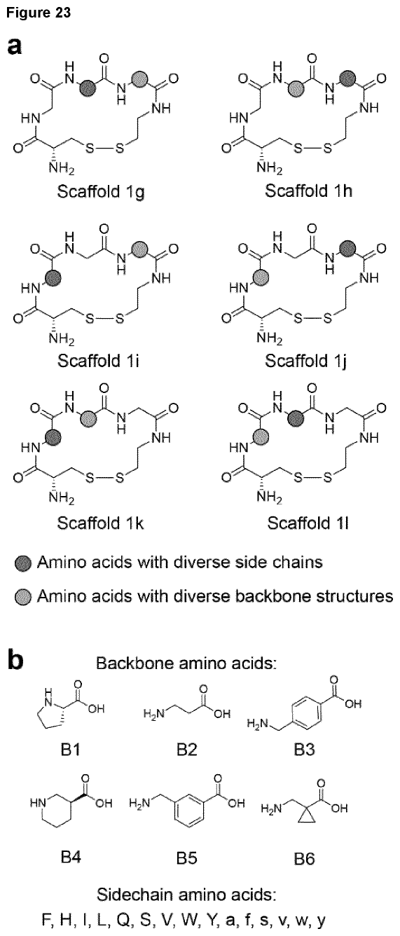

FIG. 23. Preparation of cyclic peptide scaffolds containing an N-terminal amino group. a, Six different scaffold formats. b, Amino acids used for scaffold synthesis.

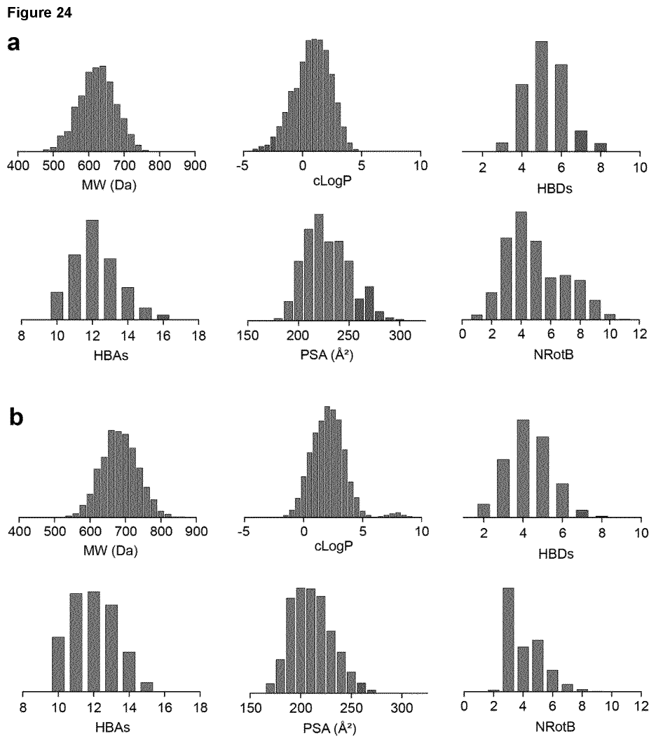

FIG. 24. Physicochemical properties of Library 1 (thrombin screen) and Library 2 (MDM2 screen). Properties were calculated using DataWarrior software. Regions compliant with Kihlberg's rules for permeability (P. Matsson et al., Adv. Drug Deliv. Rev. 2016, 101, 42-61; B. Doak et al., Chem. Biol. 2014, 21, 1115-1142) are colored green. The majority of both libraries fall in a space that is predicted to be cell permeable. MW=molecular weight, cLogP=calculated n-octanol/water partition coefficient, HBD=hydrogen bond donors, HBA=hydrogen bond acceptors, PSA=polar surface area, NRotB=number of rotatable bonds. a, Library 1 (for thrombin screen). b, Library 2 (for MDM2 screen).

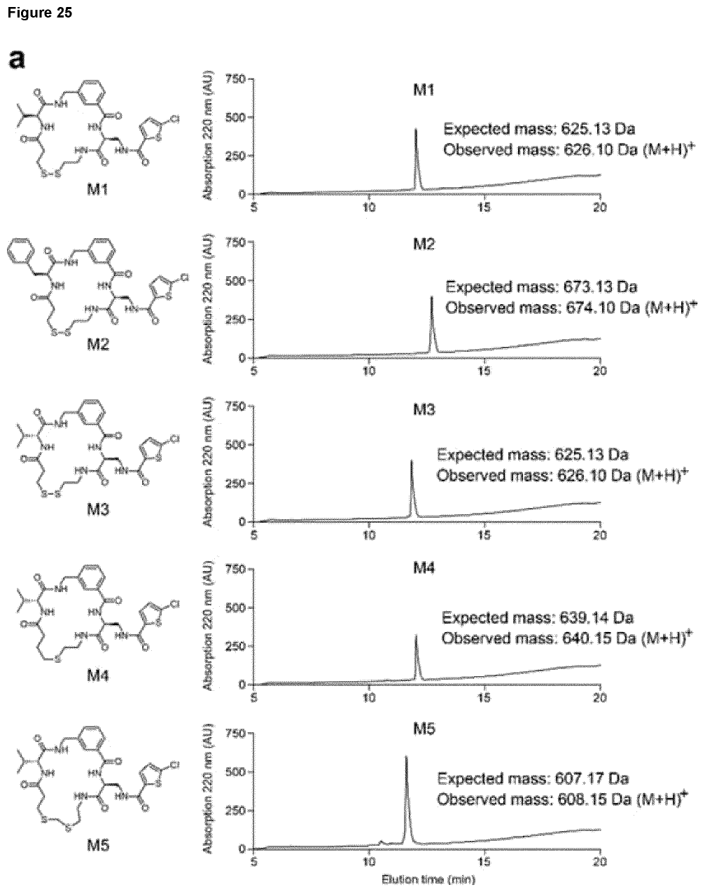

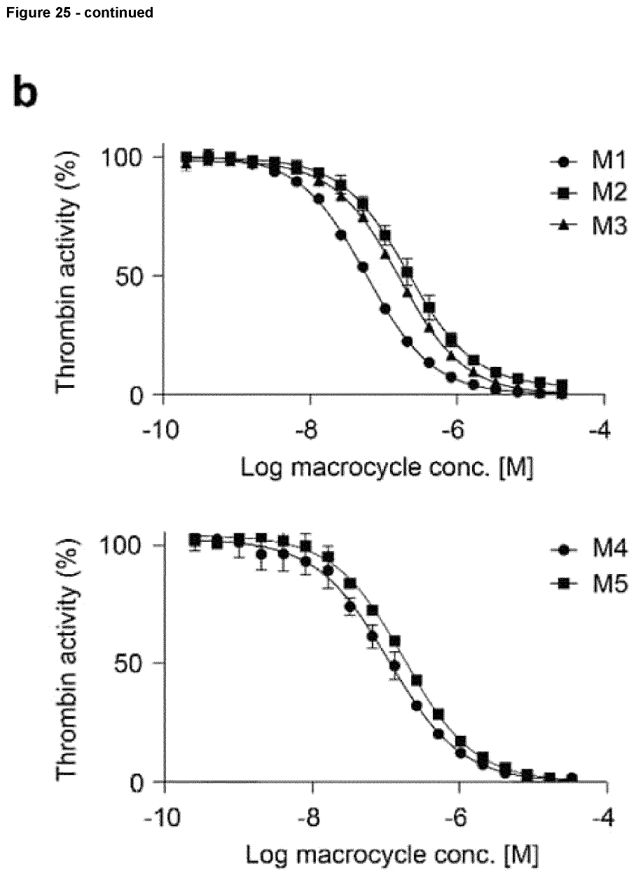

FIG. 25. Thrombin inhibitors M1 to M5. a, Chemical structures and analytical HPLC chromatograms obtained using a 0-100% MeCN/H2O gradient over 15 minutes. b, For all macrocycles, an 18-point, two-fold serial dilution was performed in 50 μl volumes. Thrombin was added (50 μl, 2 nM final conc.), followed 10 minutes later by fluorogenic substrate Z-Gly-Gly-Arg-AMC (50 μl, 50 μM final conc.). The increase in fluorescence was measured over 30 minutes. Residual thrombin activity was determined by dividing the slope of fluorescence intensity over time for each well by the slope of control wells without macrocycle. Mean values and SDs are indicated for three independent measurements.

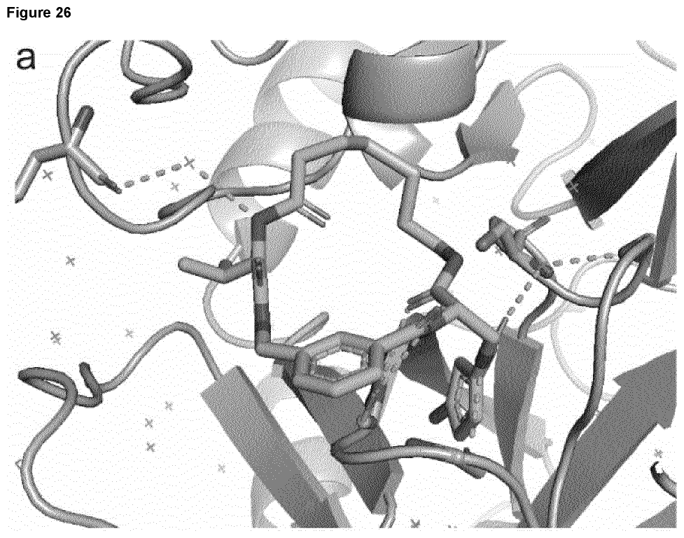

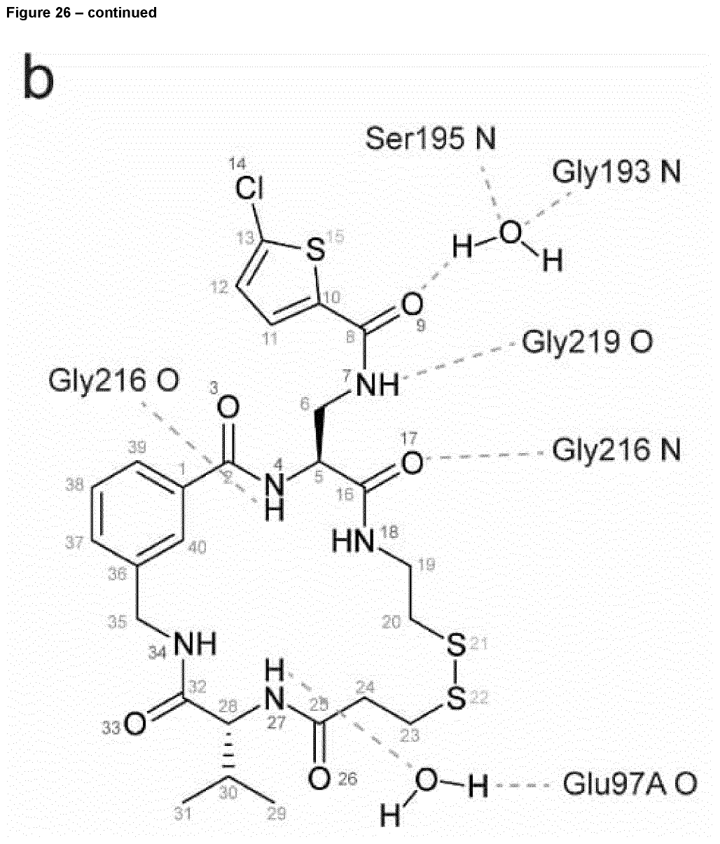

FIG. 26. Structure of M1 bound to thrombin. a, X-ray structure of M1 bound to thrombin. M1 is zoomed in and the H-bond interactions are indicated. b, Chemical structure of M1 and H-bond interactions formed with thrombin.

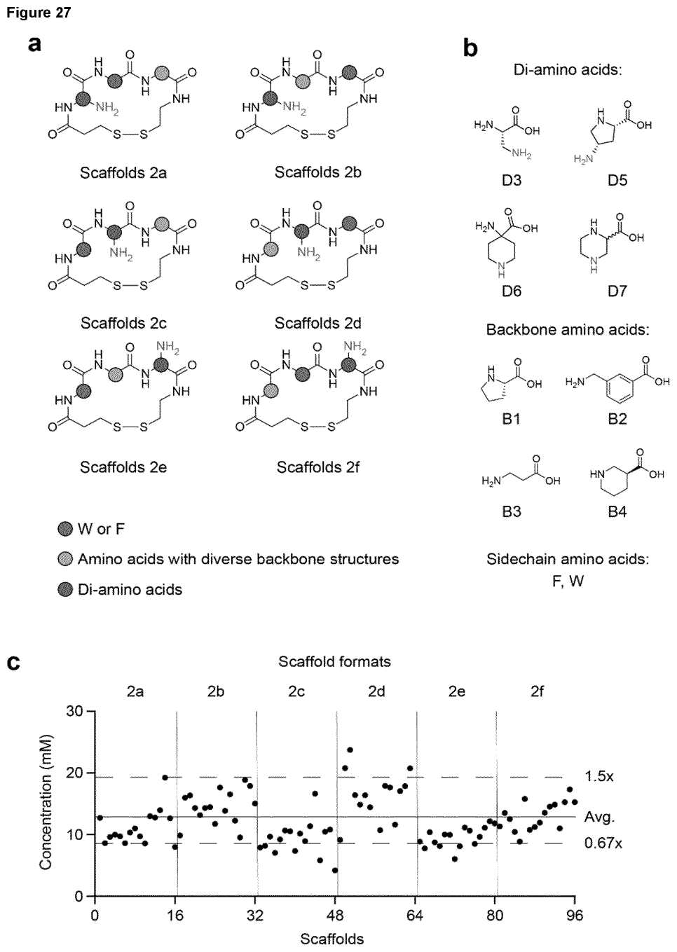

FIG. 27. Scaffolds synthesized for Library 2 (MDM2 screen). a, Format of scaffolds in Library 2. b, Amino acids used for the scaffold library synthesis. All combinations of four di-amino acids, four backbone amino acids, two sidechain amino acids, and six sub-library formats, were synthesized. c, Yields of tryptophan-containing scaffolds after cyclative release. The average concentration of scaffold was 12.9 mM as determined by nanodrop absorbance. The average purity measured by LC-MS was around 90%.

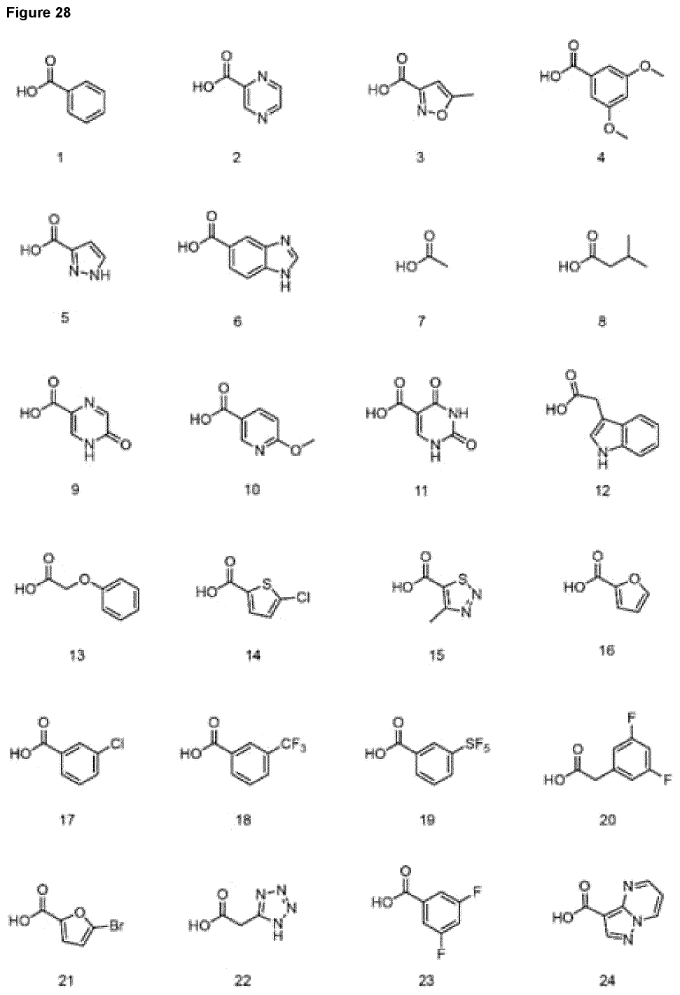

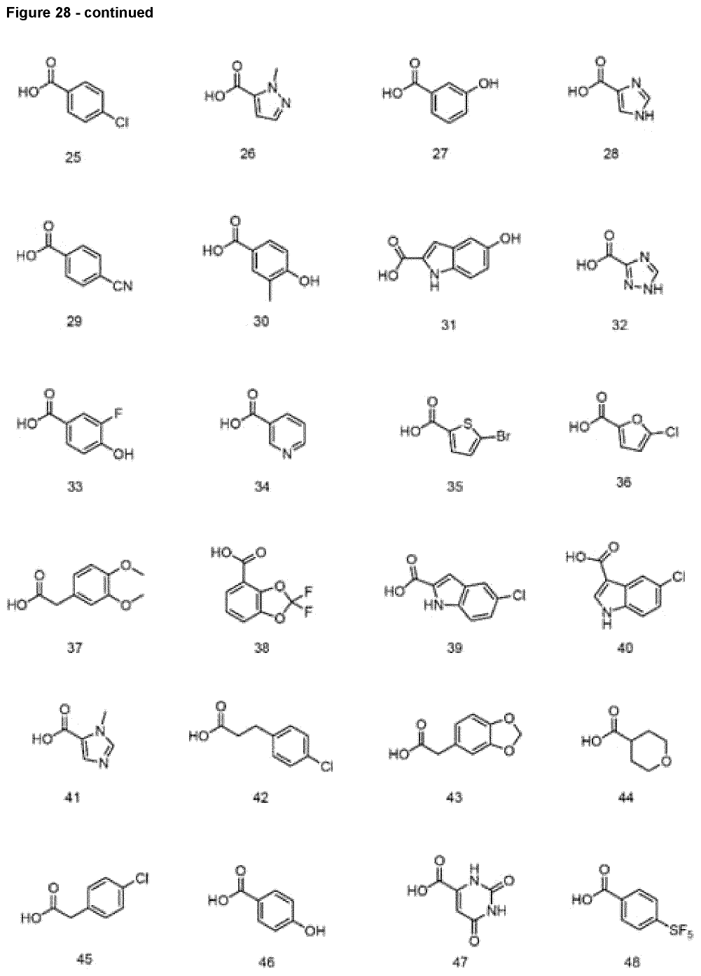

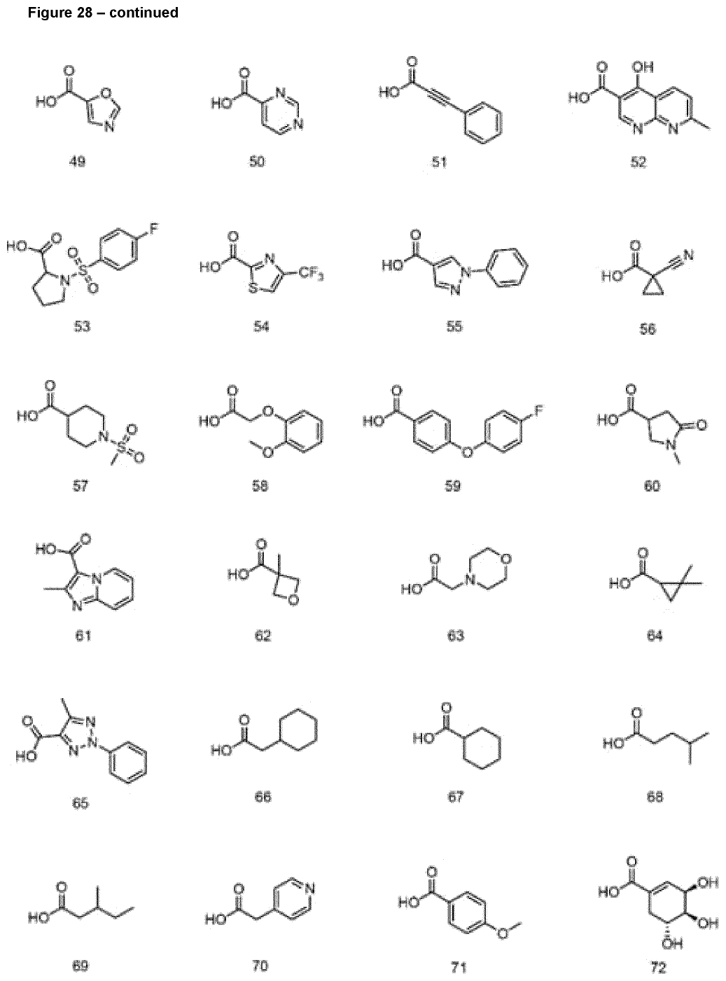

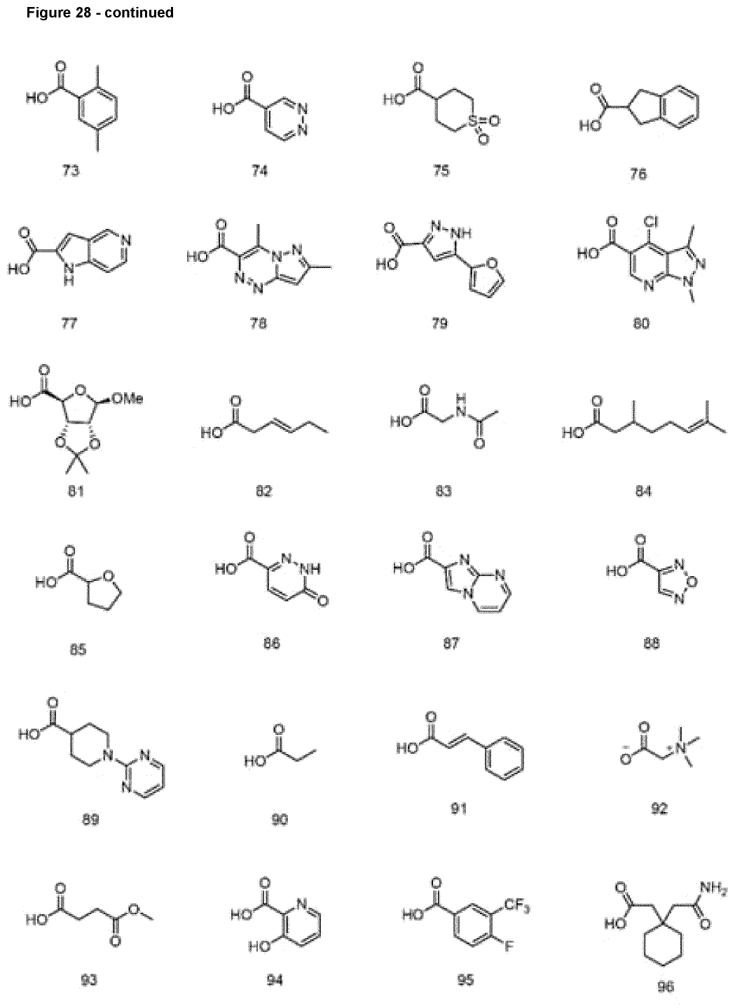

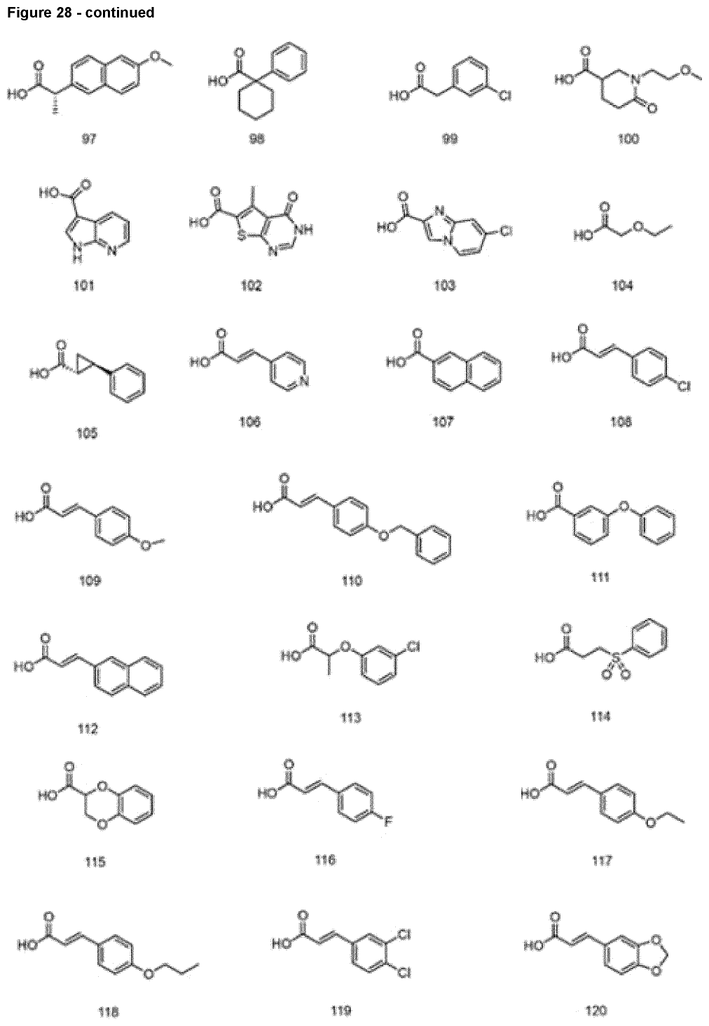

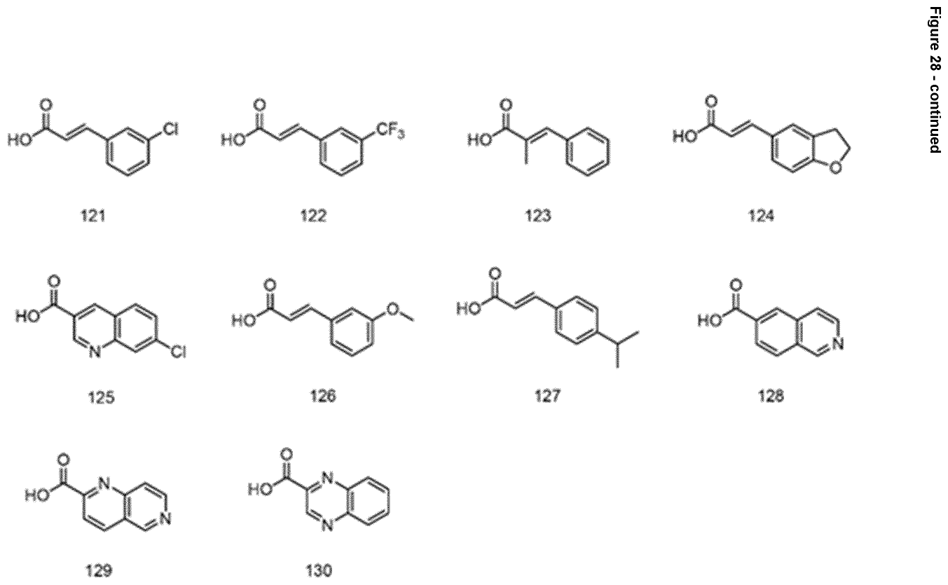

FIG. 28. Overview of carboxylic acids used to acylate peripheral amines in cyclic peptide scaffolds

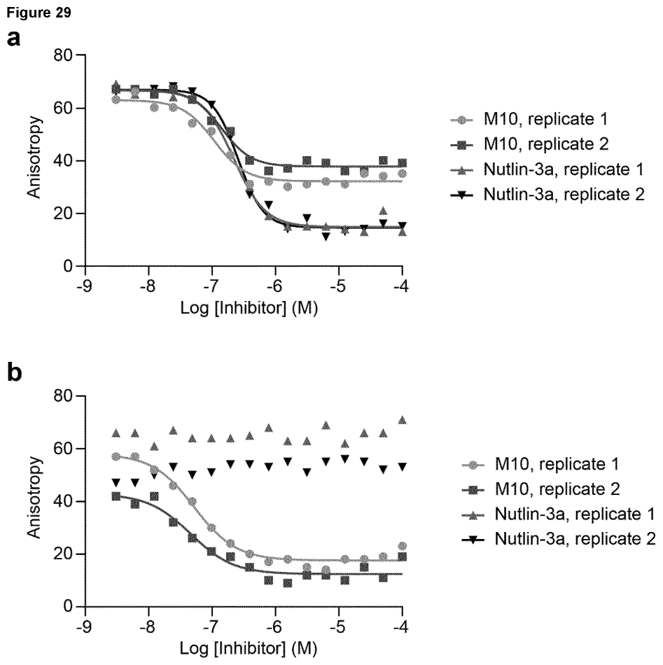

FIG. 29. Studying the binding site of M10 by competition binding experiments. a, Displacement of the FP53 linear peptide probe by M10 and nutlin-3a measured by fluorescence polarization. b, Displacement of the F-M10 macrocycle probe by M10 and nutlin-3a.

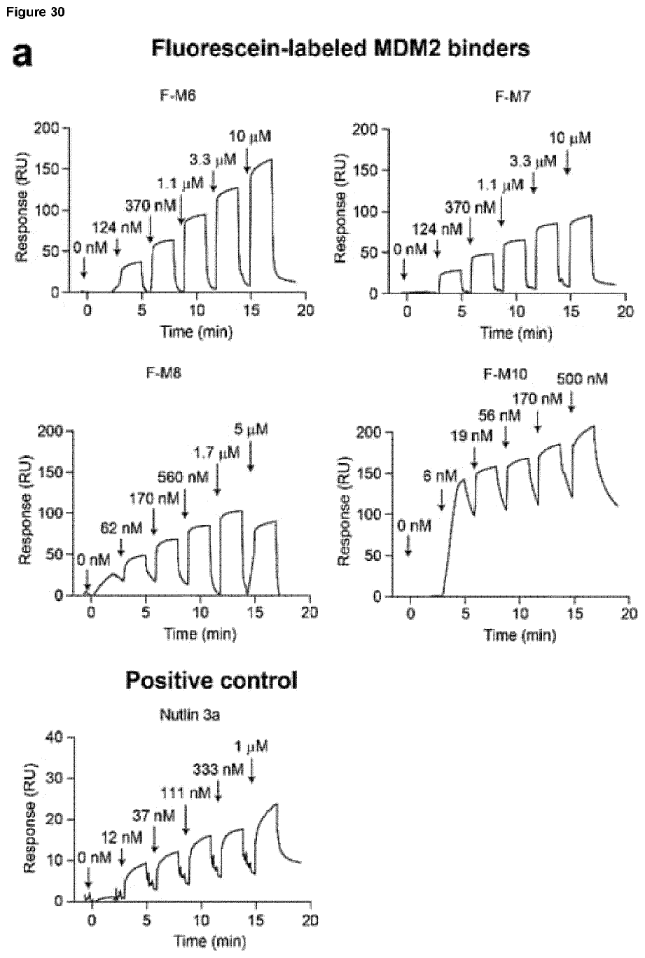

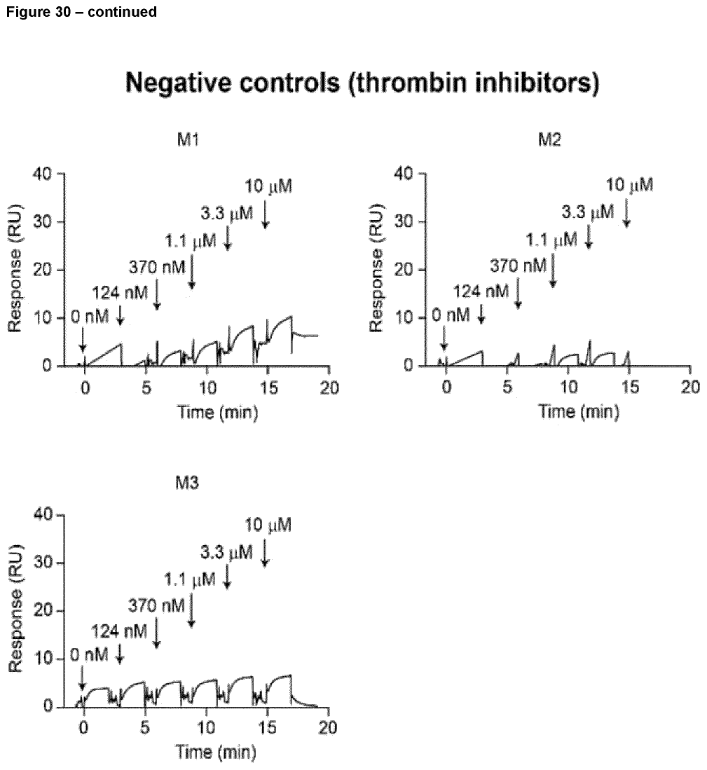

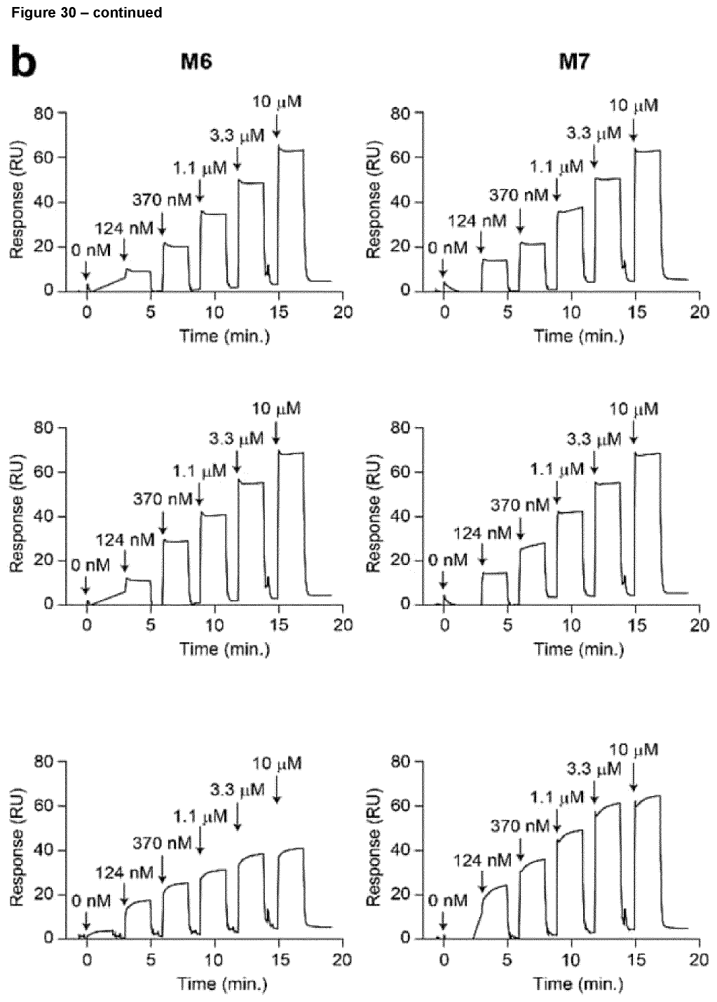

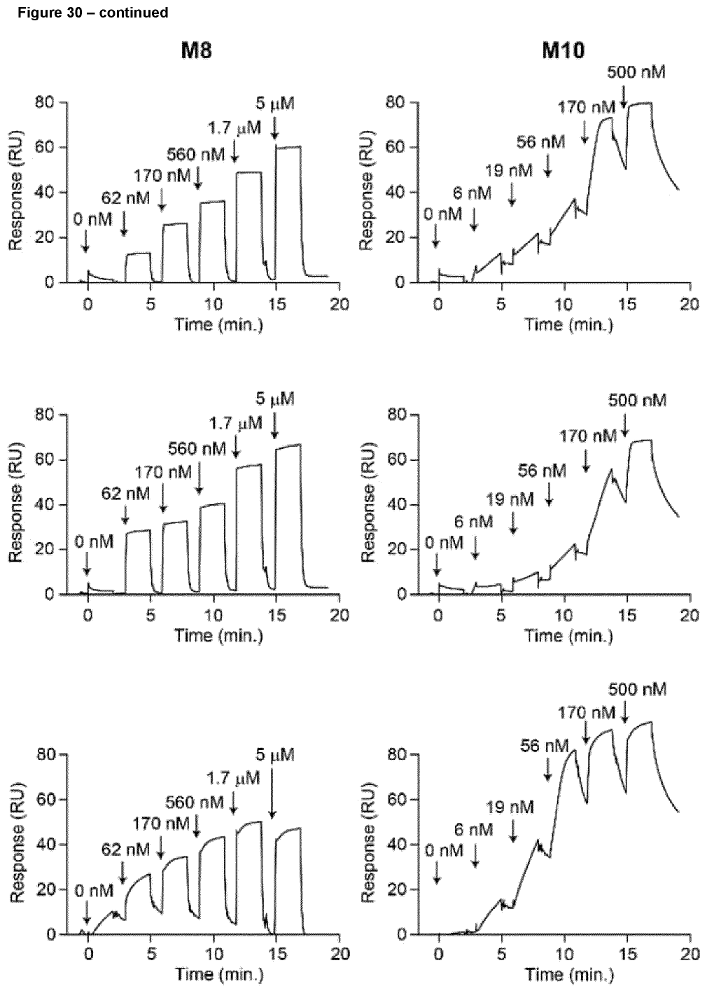

FIG. 30. Binding of macrocycles to MDM2 measured by SPR. a, Single-cycle SPR sensorgrams for fluorescein-labeled macrocycles, positive control nutlin-3a (not fluorescein labeled), and negative controls (thrombin inhibitors; not fluorescein labeled). RU, response unit. b, Single-cycle SPR sensorgrams for macrocycles M6, M7, M8 and M10 (not fluorescein-labeled), performed in triplicate.

The Examples illustrate the invention.

### Example 1—Reductive Release

### 1.1. Reductive Release of Disulfide-Immobilized Peptides from Solid Phase

It was first assessed if peptides immobilized via a disulfide bridge on solid supports could be released quantitatively with a volatile reducing agent. Towards this end, the four peptides Ala-Trp-Mea, Trp-Ala-Mea, Ala-Tyr-Mea, and Tyr-Ala-Mea (Mea=2-mercapto-ethylamine, also named cysteamine) shown in FIG. 2a were synthesized. The peptides contain a tryptophan or tyrosine residue to allow precise determination of the amount of released peptide by absorbance measurement at 220 or 280 nm. We omitted the N-terminal thiol group found in dithiol peptides to quantify peptides that were released by reduction of the disulfide-bridge and not through any other mechanism such as a cyclative disulfide exchange. The peptides were synthesized on polystyrene (PS) resin having a high loading capacity (around 1 mmol/gram) and being commonly used for peptide synthesis. First a disulfide bridge was established by incubating PS thiol resin with excess of pyridyldithioethylamine and synthesized the peptides by standard Fmoc chemistry. All peptides were synthesized in wells of a 96-well plate on a 5 μmol scale, to test the conditions at which it was planned to synthesize dithiol peptide libraries later at high-throughput.

The release of disulfide-immobilized peptide from PS resin was tested by incubating the beads overnight with 200 μl DMF containing 20 equiv. of β-Me (0.5 M) and 20 equiv. of trimethylamine (TEA; 0.5 M). LC-MS analysis of the peptides showed efficient release but also that around 40% of the product occurred as disulfide adduct with β-Me. It was reasoned that the extent of adduct could potentially be reduced by using a larger molar excess of β-Me and/or by repeating the reduction, but this would require additional working steps and thus was not the most attractive route. In order to release peptides from resins and eliminate disulfide adducts in a single step, it was proposed to use a reducing agent like dithiothreitol (DTT) which does not form disulfide adducts because it eliminates itself by forming cyclic DTT. Incubation of the resin carrying either peptide Ala-Trp-Mea or Tyr-Ala-Mea with 4 equiv. DTT efficiently released the peptides without forming peptide-reducing agent adducts, but DTT could not be removed by vacuum evaporation in a standard speedvac. A related reducing agent that evaporates at 195° C., and thus has is a lower boiling point, 1,4-butanedithiol (BDT) (PubChem, https://pubchem.ncbi.nlm.nih.gov/compound/1_4-Butanedithiol, Accessed 20.04.21). 5 μmol of the resin-linked peptides were incubated with 200 μl DMF containing 4 equiv. BDT (100 mM) and 4 equiv. TEA (100 mM). As a control, parallel reactions were performed in which the four resin-bound peptides were treated with the conditions for cyclative release, being 250 mM TEA in DMSO. The peptides were efficiently released by BDT as analyzed by LC-MS. For all the four peptides, a major peak corresponding to the desired product, was observed (FIG. 2b). The only side product found for two peptides was peptide dimer, which occurred in small quantities of less than 3%. The yields of the desired products were 3.4, 1.3, 4.2 and 4.3 μmol, respectively, which corresponded to 68, 25, 84 and 86% yield (assuming a quantity of 5 umol peptide synthesized on the beads).

### 1.2. Reductive Release of Short Dithiol Peptides from Solid Phase

Next a panel of eight short dithiol peptides of the format Mpa-Xaa-Mea (Mpa=mercaptopropanoic acid) was synthesized, wherein the Xaa amino acids were Trp, Tyr, Ser, His, Phe, Arg, Asp and Ala. For these peptides, it was expected that they are released by reduction of the disulfide bridge, but not efficiently via a disulfide cyclization mechanism. As a control, also the longer peptide Mpa-Lys-Trp-Gly-βAla-Mea was synthesized which was expected to be released efficiently via disulfide cyclization. Incubation of the resins with the reducing agent BDT led to efficient release of all peptides (FIG. 3). The main products were the desired dithiol peptides. Side products were found in only small quantities and were dithiol peptides that carried trityl and tert-butyl protecting groups (FIG. 3). Incubation of resin with TEA in DMSO for cyclative release yielded the short peptides in around 10 to 100-fold smaller quantities and lead to more side products. As expected, the longer peptide Mpa-Lys-Trp-Gly-βAla-Mea was efficiently released via the cyclative release mechanism, most likely due to the smaller conformational constraints. The yields of the peptides Mpa-Trp-Mea and Mpa-Trp-Mea that could be quantified by absorbance measurement at 280 nm were 3.6 and 4.3 μmol, respectively, which corresponded to 71% and 85% yield assuming that peptides were present on resin in a quantity of 5 μmol.

### 1.3. Evaporation of Solvent and Reducing Agent Under Vacuum

It was next tested if the reducing agent BDT could be removed by centrifugation under vacuum. Peptides synthesized at a scale of 5 μmol and released in 200 μl DMF containing 100 mM BDT and 100 mM TEA were centrifuged at 0.1 mbar and 30° C. and at 400×g in wells of 96-well plates. The solvent was efficiently removed in one hour if all wells of the microwell plate were filled. LC-MS analysis of the peptides showed that all BDT was removed. However, it was also found for some of the peptides that a fraction of up to 10% of the product was back-oxidized. It was speculated that the oxidation was enabled by the high pH, and thus 2 equiv. of TFA relative to TEA (200 mM TFA) were added to each well prior to the centrifugal vacuum evaporation. With this procedure, the fraction of oxidized peptide could be suppressed efficiently.

### 1.4. Cyclization of Dithiol Peptides by Bis-Electrophile Reagents

It was tested if the short dithiol peptides Mpa-Trp-Mea and Mpa-Tyr-Mea could be cyclized by bis-electrophile reagents as shown in FIG. 4a. The cyclization of peptides via two or three cysteines by electrophilic linker reagents is highly efficient and clean if performed at dilute concentrations, with the peptides around 1 mM (or lower) and the cyclization reagents applied at a small excess (P. Timmerman et al., ChemBioChem, 2005, 6, 821-824; S. S. Kale et al., Nat. Chem., 2018, 10, 715-723). The peptides were dissolved in 1 ml acetonitrile:water 1:1, added 3 ml of 90% NH₄HCO₃ buffer (100 mM, pH 8.0), 10% acetonitrile, and immediately added 1 ml bis-electrophile reagents in acetonitrile (10 mM). In total the ten bis-electrophile reagents shown in FIG. 4b were tested. The final concentrations of peptide and cyclization reagent were around 1 mM and 2 mM, respectively. The HPLC chromatograms of the cyclization reactions with the peptide Mpa-Trp-Mea are shown in FIG. 4c. In most of the reactions, the dithiol peptides were cyclized nearly quantitatively with yields higher than 90%. The small quantities of side product were mainly peptides that reacted with only one thiol group because one of them was protected by trityl. For quenching excess of bis-electrophile reagents, 6 equiv. (relative to peptide) of β-Me were added which reacted with these reagents but did not affect the macrocycles.

### 1.5. Discussion

Herein a solid phase peptide synthesis and elution strategy was established that delivers dithiol peptides in high purity and at a high concentration so that they can readily be cyclized with bis-electrophile reagents to synthesize macrocyclic compounds and libraries. Key elements in the strategy are i) the synthesis of the peptides via a disulfide linker, i) the deprotection of the side chains on solid phase, iii) the release of the peptides by disulfide reduction, and iv) the use of a volatile reducing reagent that can be removed by evaporation so that cyclization with bis-electrophiles is possible. The omission of purification steps after the synthesis of the dithiol peptides and the cyclization by bis-electrophile reagents enables access to large numbers of macrocyclic compounds and their application for high-throughput screening.

### 1.6. Materials and Methods

### Synthesis of 2-(2-pyridyldithio)ethylamine hydrochloride

To a stirring solution of 2,2′-dipyridyldisulfide (4.41 g, 20 mmol) in MeOH (16 ml with 2% [v/v] AcOH) was added cysteamine hydrochloride (1.14 g, 10 mmol) dissolved in MeOH (10 ml, with 2% [v/v] AcOH drop wise which is dissolved in 10 ml of MeOH with 2% (v/v) AcOH over 15 min. The reaction mixture was stirred overnight at room temperature (RT) and concentrated under reduced pressure to afford yellow color greasy oil. The residue was dissolved in MeOH (16 ml), distributed to eight 50-ml falcon tubes, and precipitated by addition of ice-cold diethyl ether (48 ml to each tube). The tubes were incubated at −20° C. for 30 min and centrifuged at 3400×g (4000 rpm on a Thermo Scientific Heraeus Multifuge 3L-R centrifuge with a Sorvall 75006445 Rotor, radius=19.2 cm; explosion proof) at 4° C. for 30 min. The product was afforded as a colorless product after repeating the precipitation 8 times (yield=90%).

### Preparation of Cysteamine-Polystyrene Resin

The following procedure describes the preparation of polystyrene resin carrying around 2 mmol cysteamine immobilized via a disulfide linker, which is sufficient for the synthesis of 4×96 peptides at a 5 μmol scale. Into each one of four 20 ml plastic syringes was added 563 mg resin (Rapp Polymere Polystyrene A SH resin, 200-400 mesh, 0.95 mmol/gram loading). The resin was washed with DCM (15 ml), then swelled in MeOH/DCM (7:3; 15 ml) for 20 min. Pyridyl-cysteamine disulfide (2.10 grams, 9.42 mmoles, 4.4 equiv.) was dissolved in MeOH (23 ml) and then DCM (53 ml). Then N,N-diisopropylethylamine (DIPEA; 410 μl) was added. A volume of 19 ml of this solution was pulled into each syringe and the syringes were then shaken at RT for 3 hours. The pyridyl-cysteamine solution was discarded and the resin was washed with MeOH/DCM (7:3; 2×20 ml), then with DMF (2×20 ml). The resin was combined into a single syringe as a suspension in DMF, washed with a solution of 1.2 M DIPEA in DMF (11.8 ml) for 5 min to ensure that all amines were neutralized. This solution was discarded, and the resin was washed with DMF (2×20 ml), then with DCM (4×20 ml), then kept under vacuum overnight to yield a free-flowing powder.

### Fmoc Peptide Synthesis in 96-Well Plates

Peptides were synthesized at a 5 μmol scale in 96-well peptide synthesis filter plates (Orochem, cat. #OF1100) using an automated peptide synthesizer (Intavis MultiPep RSi). Cysteamine-PS resin (around 5 mg, 0.95 mmol/g, 5 μmol scale) was distributed as powder to each well of the plate. The resin was washed with DMF (3×225 μl). In this and all the following washing steps, the resin was incubated for 1 min. The following reagents were transferred to tubes in the indicated order, mixed, incubated for 1 min, transferred to the resin in the microwell plate, and incubated for 45 min without shaking. Reagents: 50 μl HATU (500 mM in DMF, 5 equiv.), 5 μl N-methylpyrrolidone (NMP), 12.5 μl of N-methylmorpholine (NMM in DMF, 4 M, 10 equiv.) and 53 μl of amino acid (500 mM in DMF, 5.3 equiv.). The final volume of the coupling reaction was 120.5 μl and the final concentrations of reagents were 208 mM HATU, 415 mM NMM and 220 mM amino acid. Coupling was performed twice. The resin was washed with DMF (1×225 μl). Unreacted amino groups were capped by incubation with 5% acetic anhydride and 6% 2,6-lutidine in DMF (100 μl) without shaking for 5 min. The resin was washed with DMF (8×225 μl). Fmoc groups were removed by incubation twice DMF (120 μl) containing 20% (v/v) piperidine without shaking for 5 min each. For the synthesis of longer peptide sequences, the incubation time was reduced from 5 min to 2 min, in order to reduce exposure to the base. The resin was washed with DMF (8×225 μl). At the end of the peptide synthesis, the resin was washed with DCM (2×200 μl).

### Peptide Side Chain Deprotection in 96-Well Plates

For removing protecting groups from amino acid side chains as well as from Mea, the bottom of the 96-well synthesis plate was sealed by pressing the plate onto a soft 6 mm thick ethylene-vinyl acetate pad, and the resin in each well was incubated with a solution of TFA:TIPS:H2O (95:2.5:2.5 [v/v/v], around 300 μl). The plates were covered with an adhesive sealing film (IST scientific, QuickSeal Micro, cat. #IST-125-080-LS), then weighed down by placing a weight (1 kg) on top to prevent leakage. After 1 h incubation, the synthesis plate was placed onto a 2 ml deep-well plate, and the TFA mixture was allowed to drain. The synthesis plate was again sealed and the deprotection procedure was repeated. The wells were washed with DCM (3×500 μl; added with syringe) that was run through the wells by gravity flow. The resin was dried by placing the synthesis plate into a vacuum manifold for 5 min.

### Reductive Peptide Release by β-Me, DTT or BDT

For releasing the peptides from the resin, the bottom of the 96-well synthesis plate was sealed by pressing the plate onto a soft 6 mm thick ethylene-vinyl acetate pad, and the resin in each well was incubated with a solution of 200 μl DMF containing 500 mM of β-Me, or 100 mM DTT, or 100 mM BDT, and 100 mM TEA for 4 h at RT. After this time, the samples were collected in a 96-deep well plate by centrifugation at 250×g (1100 rpm on a Thermo Scientific Heraeus Multifuge 3L-R centrifuge with a Sorvall 75006445 Rotor, radius=19.2 cm rotor) for 2 min at RT.

### Cyclative Release of Peptides

The 96-well synthesis plate was sealed as described above and the peptides were released from the resin by incubation with a solution of 200 μl DMSO containing 250 mM TEA (10 equiv.) overnight at RT. After this time, the samples were collected in a 96-deep well plate by centrifugation at 250×g (1100 rpm on a Thermo Scientific Heraeus Multifuge 3L-R centrifuge with a Sorvall 75006445 Rotor, radius=19.2 cm rotor) for 2 min at RT.

### LC-MS Analysis of Peptides After Solid Phase Release or Cyclization

For peptides released from solid phase (concentration up to 25 mM in DMF or DMSO), 1 μl of peptide was diluted in 60 μl of milliQ H2O containing 0.05% formic acid. For peptides from cyclization reactions (concentration around 1 mM), 10 μl of the reaction mixture was mixed with 10 μl of milliQ H2O containing 0.05% formic acid. Samples (10 μl injection) were analyzed on a Shimadzu 2020 single quadrupole LC-MS system using a reverse phase C18 column (Phenomenex Kinetex®, 2.6 μm, 100 Å, 50×2.1 mm) and a linear gradient of solvent B (MeCN, 0.05% formic acid) over solvent A (H2O, 0.05% formic acid) from 0 to 60% in 5 min at a flowrate of 1 ml/min. Absorbance was recorded at 220 nm and masses were analyzed in the positive mode.

### Vacuum Centrifugal Evaporation of Reducing Agent and Solvent

The following example describes a peptide that had a concentration of 20 mM after reductive release. Of the 200 μl peptide released from the solid phase by reduction (in DMF containing 100 mM BDT and 100 mM TEA), 5 μl (0.1 μmol) were transferred to a well of a V-bottom 96-well plate (Ratiolab, 6018321, PP, unsterile). A volume of 7 μl of 1% TFA in water (v/v) was added to each well to reach 2 equiv. of TFA over TEA. This sample was subjected to vacuum centrifugal evaporation using Christ RVC 2-33 CDplus IR instrument to remove the solvent (DMF) and reducing agent (BDT). Samples were centrifuged at 0.1 mbar, 30° C. and at 400×g (1750 rpm in a Christ 124700 rotor with 124708 plate holder inserts, radius=10.5 cm). The peptides were not visible after this step.

### Cyclization of Peptides

The reduced and dried peptide (0.1 μmol) was dissolved in 20 μl of 50% acetonitrile, 50%H2O to reach a concentration of 5 mM. To this solution 60 μl reaction buffer (100 mM ammonium bicarbonate, pH 8.0, containing 10% acetonitrile [v/v]) was added followed by 20 μl of 10 mM cyclization linker in acetonitrile (2 equiv.). The final concentrations in the reaction were 1 mM peptide, 2 mM cyclization linker, 60 mM ammonium bicarbonate buffer and 35% acetonitrile. The plate was covered with a foil seal and the reaction incubated for 2 h at RT.

### Quenching of Linker Reagents in Cyclization Reactions

After completion of the cyclization reaction, 4 μl of 150 mM β-Me in acetonitrile (0.6 mol, 6 equiv. relative to the peptide) was added to the reaction mixture and incubated at for 1 h at RT. The solvent (MeCN), buffer (bicarbonate) and excess β-Me were removed by vacuum centrifugal evaporation using Christ RVC 2-33 CDplus IR instrument. Samples were centrifuged at 0.1 mbar, 30° C. and at 400×g (1750 rpm in a Christ 124700 rotor with 124708 plate holder inserts, radius=10.5 cm). The peptides were not visible after this step.

### Example 2—Cyclative Release

### 2.1. Results and Discussion

In a first experiment, different resins were tested for the synthesis of short peptides that were tethered via a disulfide bridge to a solid-phase. The peptide Mpa-Gly-GIn-Trp-Mea was synthesized on five different resins, wherein Mpa is mercaptopropanoic acid (cysteine without the amino group) and Mea is 2-mercapto-ethylamine (cysteamine; cysteine without carboxylic acid) (FIG. 5b). Two polyethylene glycol (PEG) resins that are polar (1, 2), one PEG-modified polystyrene (PS) resin that is polar too, and two PS resins that are apolar (4 and 5) were used. Resin 5 contained already a thiol group and for resins 1 to 4 a thiol group was introduced through appending trityl-protected Mpa through amidation. The disulfide-linked Mea was introduced by incubating the resins with excess of 2-(2-pyridinyldithio)-ethanamine. The disulfide-exchange reaction was tested in a methanol (MeOH)/dichoromethane (DCM) mixture and in DMF, either in presence of a base or an acid, and found that the condition in 30% MeOH, 70% DCM and one equiv. N,N-diisopropylethylamine (DIPEA; relative to 2-(2-pyridinyldithio)-ethanamine) worked best. The three amino acids Trp, Gln and Gly, and Mpa were appended using standard Fmoc chemistry, and the side chain protecting groups removed by incubation of the resins with 95% TFA, 2.5% TIS and 2.5% water for one hour.

Next the cyclative disulfide release was tested by deprotonating the sulfhydryl group at the N-terminal end of the peptide using DIPEA as a base. Incubation of the resins in DMSO with 150 mM DIPEA led to highly efficient release in case of the apolar resins 4 and 5 (FIG. 5c). The concentrations of the peptide in the eluate were 5.1 mM (resin 4) and 11.6 mM (resin 5), respectively. The quantities of released peptide were 21±5% (resin 4) and 44±4% (resin 5) of that expected to be synthesized based on the resin loadings. Given that the disulfide linker installation was most likely not quantitative, even for the resin that carried already thiol groups (resin 5), the percentage of peptide recovered by the cyclative release mechanism was likely higher than 44%. LC-MS analysis of the products showed high purities of 95±4% (resin 4) and 93±1% (resin 5) for the disulfide cyclized peptide (FIG. 5d). The only side product was cyclic peptide dimer and was found in only small quantities of 6% in average. The dimeric cyclic product was most likely formed by disulfide exchange-mediated transfer of one peptide to a neighboring one on resin and subsequent cyclative release of a cyclic dimer.

To assess the substrate scope of the cyclative disulfide release strategy, four peptides of the format Mpa-Gly-Ala-Xaa-Mea at a 25 μmol scale were synthesized (FIG. 6a). In the position “Xaa” of three of the four peptides, amino acid building blocks were inserted that imposed rigidity into the peptides' backbones. HPLC analysis of products obtained by base-induced cyclative release showed a dominant peak for each one of the four peptides, and MS analysis confirmed that these main products were the desired cyclic peptides. Absorbance measurement showed that all four peptides were obtained in double-digit millimolar concentrations. Quantification of the product by weighting after excessive lyophilization revealed yields ranging from 13.5 to 26 μmol, which corresponds to yields between 54% and 100% of those expected based on the resin loading. For all four peptides, only a limited number of side products were observed and they were found in small quantities (FIGS. 6b and 6c). The main side products were cyclic dimers, this time eluting as two close peaks, which corresponded most likely to the two possible dimers, one linked head-to-head/tail-to-tail and one head-to-tail. Linearization of the products with TCEP and HPLC analysis showed linear peptide products with purities of 96, 96, 100 and 92% for peptides 1-4, respectively (FIG. 6b, right chromatograms). The TCEP-linearization experiment suggested that dimeric cyclic peptide can be removed by reduction and subsequent oxidative re-circularization at concentrations that favor intramolecular cyclization. In order to test if even shorter peptides could be generated following the cyclative release strategy, the experiment was repeated with four peptides of the format Mpa-Ala-Xaa-Mea and thus being one amino acid shorter. The four peptides were efficiently released too and the main peak in the HPLC profile was the desired cyclic peptide. The fraction of cyclic dimer was slightly higher overall, most likely due to the less efficient circularization that was hindered by the short backbone and the resulting conformational constraints.

In order to assess if the cyclative disulfide release strategy can be applied for library synthesis and screening, cyclic peptides were designed and synthesized in a 96-well plate and at a 5 μmol scale. 96 random disulfide-cyclized peptides of the three formats were prepared shown in FIG. 7a. The peptides contain three random amino acids Xaa flanked by Mpa and Mea. Two of the random amino acids in each peptide were selected from four structurally diverse amino acids that lead to highly diverse cyclic peptide backbones. One of the random amino acids was Trp or Tyr that allowed quantification of peptide yields by absorption measurement at 280 nm. Cyclative release of the peptides by addition of 150 mM DIPEA in 200 μl DMSO and subsequent absorption measurement showed a high average peptide concentration of 13.3 mM and a narrow concentration distribution for 90 of the peptides that were between 8.9 mM (1.5-fold below average) and 20 mM (1.5-fold above average). Three of the peptides were not synthesized or released at all. Analysis of 12 randomly picked cyclic peptides by LC-MS showed a high purity of 84% in average.

Despite the relatively small number of only 96 cyclic peptides and thus small chance of finding an active compounds, we performed a screen against thrombin, an important target for developing anticoagulation therapeutics, using a compound concentration of around 10 μM. The most active peptide reduced the thrombin activity 52% which was remarkable considering that peptides did not contain the amino acids Arg and Lys that bind to the thrombin specificity pocket S1 (FIG. 7c). Repetition of the screen identified the same cyclic peptide as the most active hit (green dots in FIG. 7c). The HPLC-purified disulfide cyclized peptide Mpa-Tyr-II-Pro-Mea inhibited thrombin with a Kᵢ of 13±1 μM. The small-scale screen showed that most of the cyclic peptides did not affect the thrombin activity, suggesting that there was no component eluted with the peptides that interfered with the biological assay, including the DMSO and the DIPEA that are present in the peptide stocks after cyclative release. Biological screens are typically performed at compound concentrations of around 10 μM, which means that the 100% DMSO and 150 mM DIPEA in the around 10 mM peptide stocks get 1000-fold diluted to reach concentrations of 0.1% and 150 μM, respectively, that unlikely affect most biological assays.

### 2.2. Conclusion

In summary, a cyclative peptide release strategy was developed herein based on a disulfide exchange reaction that yields disulfide-cyclized peptides in high purity directly from the solid support. To our knowledge, this is the first approach in which cyclic peptide libraries are released in a high purity and with a cleavage reagent that can be removed by evaporation so that the peptides can readily be screened using bioassays without prior purification. Importantly, the yields of peptides with different sequences showed a narrow distribution, allowing the screening of the cyclic peptides even without determining or adjusting the concentrations. It is shown that the approach is applicable for the generation of libraries comprising hundreds of peptides.

### Example 3—Massive Expansion of Cyclic Peptides Library Size

### 3.1. Acylation of Cyclic Peptide Scaffolds Via Peripheral Amines

For combinatorially diversifying macrocycle libraries as shown in FIG. 8a, it was chosen to modify amines that serve as peripheral groups, and fragments that are carboxylic acids. N-acetylation reactions are efficient and selective, and have been broadly applied in the synthesis of DNA-encoded chemical libraries (P. R. Fitzgerald et al., Chem. Rev., 2020). N-acetylation was also used for diversifying lead structures by a panel of carboxylic acids in solution, followed by activity screening crude reactions (A. Brik et al., Chem. Biol., 2002, 9, 891-896) or even X-ray crystallographic analysis (M. R. Bentley et al., J. Med. Chem., 2020, 63, 6863-6875). The reaction was tested with the model scaffold cyclo(Mea-Lys-Xaa-Mpa), carrying a primary amine as a peripheral group (FIG. 8b), and eight structurally diverse carboxylic acids (FIG. 8c). In order to efficiently convert the scaffolds into the desired products, it was chosen to react them with a 4-fold molar excess of carboxylic acid. Near-quantitative conversion of scaffold into product was desired as non-modified scaffold could potentially bind weakly and interfere in the screen, in case it was more abundant than the acylated scaffold. The excess of carboxylic acid leads to non-reacted carboxylic acid that would be present in the screen, but it was reasoned that most carboxylic acids on their own would not bind to the target due to their small size. The only byproducts of the acylation reaction expected were excess carboxylic acid, HOBt, and tetramethylurea, none of which should be incompatible with biochemical assays. The cyclic peptide scaffold (10 mM final conc.) was incubated with a 4-fold molar excess of the eight acids in volumes of 4 μl using HBTU as activating agent and DIPEA as base for three hours, and found full conversion of the scaffold with nearly all acids (top numbers in FIG. 8c).

### 3.2. Macrocycle Library Synthesis in Nanoliter Volumes

The combinatorial diversification of the same cyclic peptide scaffold was subsequently tested in 80 nl, and thus a 50-fold smaller volume. This step was essential as it was planned to generate the libraries at a nanomole scale in small volumes so that micromole quantities of scaffold, that could easily be synthesized in wells of 96-well plates (5 μmol scale), was sufficient to synthesized more than 100 macrocycles from one scaffold. In addition, it was aimed at applying acoustic dispensing technology for transferring reagents, which is suited to transfer nanoliter volumes but not microliter ones. Acoustic dispensing has the great advantage that reagents can be transferred contact less, which does not require pipetting tips, accelerating the speed of dispensing and reducing waste and cost. Application of the same reaction conditions led to much lower yields (first of the two lower numbers in FIG. 8c) and called for optimization of the acylation reaction. It was hypothesized that the low yields were related to the use of DIPEA, as the base is relatively volatile and got lost partially when being dispensed in 2.5 nl droplets by acoustic waves. Tests were conducted with the non-volatile bases DABCO and the sodium salt of HEPES, and the volatile NMM that has a high solubility in the solvent system used and could be applied at higher concentration. With all three bases, the macrocycles were quantitatively acylated with three acids tested, and application of DABCO to further acids showed that the conditions were suited for efficient modification of peripheral amines in cyclic peptides (second of the two lower numbers in FIG. 8c).

In a next step, the acylation reactions were tested with other macrocyclic compounds. Specifically macrocyclic scaffolds were chosen in which the amino groups are less exposed than in the one of the model scaffold cyclo(Mpa-Lys-Xaa-Mea). Towards this end, 4 macrocyclic compounds offered by the company Enamine were ordered (FIG. 13a). All these compounds were non-peptide macrocyclic compounds, containing other building blocks than amino acids. Acylation reactions using the same reaction conditions and acids (FIG. 13b) showed efficient conversion of macrocycle scaffolds to the macrocycles carrying carboxylic acids (FIG. 13c).

### 3.3. Synthesis of Cyclic Peptide Scaffolds Carrying Amine Groups

Next cyclic peptide scaffolds were synthesized having random structures and one peripheral amino group, using a recently developed approach to efficiently produce large numbers of small cyclic peptides in 96-well plates. In brief, short peptides are synthesized on solid phase and released though a disulfide-cyclization reaction to yield essentially pure scaffolds that do not need further purification (FIG. 9a). A first library of cyclic peptide scaffold was generated containing three amino acids that were varied, one being an amino acid with a primary amine in the side chain (chosen from seven aa), one being an a-amino acid with a random side chain (chosen from 15 aa), and one having a random backbone structure (chosen from six aa) (FIGS. 9b and 9c; Sub-libraries 1a-f). Also a second library in which primary amino groups were introduced through cysteine residues was synthesized. Of the 3,240 (Library 1) and 540 (Library 2) different scaffolds that could theoretically be assembled in a combinatorial fashion using the indicated amino acids, 384 were randomly chosen and they were synthesized in four 96-well plates. Quantification of 45 of the cyclic peptides that contained a Trp residue by absorption showed that most molecules were obtained in good quantity (average conc.=8.1 mM; FIG. 9d). Given a relatively narrow distribution of the yields, the concentrations were not normalize for further use.

### 3.4. Combinatorial Macrocycle Library Synthesis and Thrombin Screen

The 384 scaffolds were combinatorially reacted with 12 carboxylic acids (FIG. 10a), yielding 4,608 different macrocycles and screened this library against the coagulation protease and therapeutic target thrombin. An inhibitor of thrombin is already used in the clinic as an anti-thrombosis drug but suffers from low oral availability. As carboxylic acids, structurally diverse molecules were chosen, including several fragments that could potentially bind into the S1 (H-bonding) and S2 (hydrophobic interactions) specificity pockets of thrombin (FIG. 10a). Groups containing positive charges such as guanidines are known to bind particularly well to the S1 sub-site, but they were omitted because the interest was in developing macrocycles with a limited polar surface and no charge, that could potentially be applied orally. In fact, the active form of the approved thrombin inhibitor contains such a positively charged group and needs to be applied as pro-drug, which may account for the limited oral availability. Using the acoustic dispenser, 20 nl of scaffold (8.1 mM in average) and 20 nl of pre-activated acid (80 mM) were combined to reach final concentrations of around 4 mM scaffold and 40 mM carboxylic acid (FIG. 10b). It was chosen to apply a 10-fold molar excess of carboxylic acid (versus 4-fold before) because some of the amino groups in the scaffolds are less well accessible than the α-amino group in the model peptide above. After five hours, the reaction overnight was quenched by the addition of 5 μl of 100 mM Tris buffer, added thrombin in a volume of 5 μl (2 nM final conc.) and measured the remaining thrombin activity using a fluorogenic substrate (5 μl). The concentration of macrocyclic compounds in the screen was around 10 μM. A fraction of 0.2% (9/4277) of the reactions inhibited thrombin >50%, all of them being macrocycles containing chlorothiophene acid (14) (FIG. 10c). Repetition of all macrocycle syntheses reactions that involved chlorothiophene acid (384 scaffolds×14) and the thrombin in an independent experiment identified essentially the same top hits and thus showed a high reproducibility for both, the diversification reactions, and the activity screen (FIG. 10d).

The chlorothiophene acid (14) that gave most of the hits was previously reported to serve as S1 sub-site binding group in the trypsin-like serine protease FXa (P. M. Fischer, J. Med. Chem., 2018, 61, 3799-3822), and it was thus expected that the hits were macrocycles that point the chlorothiophene group into the S1 pocket. By far not all macrocycles modified with acid 14 inhibited thrombin, indicating that the macrocycle contributed substantially to the binding. Given the 10-fold excess of carboxylic acid used in the acylation reactions, unreacted 14 was present at a concentration of around 100 μM in all wells of the thrombin screen. At this concentration, it did not inhibit thrombin, as shown by the control reaction in which 14 but no macrocyclic scaffold was added, and the numerous of the 384 wells that contained 14 and a scaffold but showed no thrombin inhibition (FIG. 10c). As 14 occurs as carboxamide after reaction with the amino groups, the inhibition of thrombin was tested by chlorothiopheneamide and found of Kᵢ=380 μM. The weak inhibition confirmed that the macrocyclic scaffolds were substantially contributing to the activity of macrocyclic compounds identified. The best three hits, M1, M2, and M3, were highly related in structure, all being based on scaffolds of the format cyclo(Mpa-D3-B5-Xaa-Mea) wherein the amino acids Xaa were all a-amino acids with hydrophobic side chains (D-Val, L-Phe, L-Val; FIG. 10e).

### 3.5. Acylated Scaffolds are the Active Species

It was next assessed if the activity observed in the screens derived from the anticipated macrocyclic compounds, or side products as for example adducts of the carboxylic acids or macrocycle dimers. Towards this end, the reactions of scaffold and carboxylic acid 14 were repeated for the top two hits M1 and M2 at a 250-fold larger scale (identical concentrations but larger volume), and the reactions run over a RP-HPLC column to separate 20 fractions each combining products that eluted in one minute, lyophilized the fractions and measured the thrombin inhibition activity (FIG. 10f). For both reactions, the fractions containing the desired macrocyclic products showed by far the highest activity, indicating that the hits were identified based on the activities of the macrocycles M1 and M2.

The purified macrocycles M1, M2, and M3 inhibited thrombin with Kᵢs of 44, 165 and 125 nM, respectively (FIG. 10e). Depending on the therapeutic application, the reducible disulfide bonds present in all scaffolds screened herein are not desired, and it was thus tested if they could be replaced by more stable bonds. M4 and M5 were synthesized that contained dithioacetal or thioether linkers. M4 and M5 inhibited thrombin with Kᵢs of 83±8 nM and 135±16 nM, and thus with only 2- and 3-fold weaker affinity.

### 3.6. Development of Protein-Protein Interaction Inhibitors

Macrocycles have received much interest for the inhibition of protein-protein interactions (PPIs), the prototype PPI disease target being MDM2:p53 for which many attempts were made to develop inhibitors (M. Konopleva et al., Leukemia, 2020, 34, 2858-2874; L. Skalniak et al., Expert Opin. Ther. Pat., 2019, 29, 151-170). Overexpression of MDM2 inhibits the activity of the tumor repressor p53, and MDM2 binders blocking the MDM2-P53 interaction are of interest for developing new anti-cancer therapies (P. Chène, Nat. Rev. Cancer, 2003, 3, 102-109). To test the new approach with this challenging PPI target, 192 structurally diverse cyclic peptide scaffolds were synthesized, all based on three random amino acids of which one contained an amino group for lateral diversification. In order to increase the chances of identifying binders, in all scaffolds either tryptophan or phenylalanine was included, two amino acids that form key interactions in stapled peptides that bind MDM2 and inhibit the MDM2:p53 interaction. The cyclic peptide scaffolds were synthesized in 96-well plates as described for the Library 1 above and were obtained in an average concentration of 12.9 mM and an average purity of 90%. As before, the scaffolds were modified by acylation with fragments in a combinatorial fashion, this time using 104 carboxylic acids. The size of the scaffold library was thus expanded from 192 structures to 19,968 macrocyclic compounds, and thus by more than a factor 100.

The library was screened by dispensing to the reactions in the 384-mictrowell plates the target protein MDM2 and a reporter peptide to measure binding of the macrocycles. The fluorescent reporter peptide binds to the PPI interface on MDM2 (K_{d}=0.5 μM) and its displacement by macrocycles can be followed by measuring fluorescence polarization. The screening result was displayed again in an array of scaffold (vertical) and fragment (horizontal) combinations with the color indicating the extent of reporter peptide displacement from MDM2 (FIG. 11). Two groups of hits appeared most interesting, one found on a horizontal and thus macrocycles sharing the same scaffold, cyclo(Mpa-Trp-D3-B4-Mea), and one found on a vertical line, and thus macrocycles sharing the same carboxylic acid (9). Repetition of the acylation reaction and screening, using all 192 scaffolds and four carboxylic acids that gave the best hits (9, 14, 25, 91), confirmed the results of the initial screen. An analysis of the active species in three reactions of each of the two hit groups revealed that for the first group (hits M6, M7 and M8), the active species were the anticipated macrocyclic structures, but not for the second group (FIG. 12a).

### 3.7. Identification of Nanomolar MDM2 Binders

The purified macrocycles M6, M7 and M8 displaced the fluorescent peptide probe from MDM2 efficiently and with similar IC₅₀ values around 1 μM (FIG. 12b), but the competition assay was not suited to determine binding constants in the low micromolar or nanomolar range due to the high MDM2 concentrations needed in the assay (1.2 μM). The three macrocycles were, thus, synthesized as conjugates with fluorescein that was linked to the N-terminal region of the peptide scaffold and measured the binding affinities in a direct fluorescence polarization assay (FIG. 12c). The conjugates showed K_{d} values of 650±50 nM (F-M6), 790±80 nM (F-M7), and 340±40 nM (F-M8).

### 3.8 Iterative Picomole-Scale Synthesis and Screening

The facile synthesis of macrocyclic compounds allows for the iterative synthesis of sub-libraries based on hit compounds that can improve binding affinity. To enhance the potency of macrocycle M8 that we identified in the initial screen, we synthesized 63 scaffolds (FIG. 18a) using similar amino acids to those in M8, which were three analogs of nipecotic acid (Nip), three analogs of diamino propionic acid (Dap), and seven analogs of tryptophan (Trp) (3×3×7=63). We then diversified the 63 scaffolds with 14 carboxylic acids, which included repeats from the initial library hits (carboxylic acids 14, 25, 91), as well as related structures that were analogs of cinnamic acid 91, the carboxylic acid of the macrocycle hit M8 (105-115; FIG. 28). To identify binders with nanomolar affinity, we screened the 882 macrocycles (63×14) at a 13-fold lower concentration (750 nM) than in the first screen, which corresponded to a 30 pmol scale (FIG. 18b). While the most active macrocycles were based on the original scaffold, we identified carboxylic acids in this screen that yielded more potent macrocycles, namely 109 that displaced the reporter peptide by 68% at 750 nM (macrocycle M9), compared to 21% for M8 (acid 91; FIG. 18b). Although we did not improve upon the scaffold, we gleaned meaningful structure-activity relationship data for the macrocyclic ring from the screen with 63 scaffold and showed that all building blocks were essential.

We subsequently performed a third cycle of library synthesis and screening, where we acylated the nine most promising scaffolds of the previous screens with 15 additional carboxylic acids, which were mostly cinnamic acid derivatives with larger substituents. We subsequently identified M10, which is a macrocycle based on the original scaffold and acylated with acid 120 that displaced the fluorescent probe used, F-M8, to 84% from MDM2 at 750 nM, and more efficiently than the parent compounds, M8 and M9 (FIG. 18c). We conjugated the macrocycle M10 to fluorescein and measured its binding to MDM2 using FP for a Kd of 43±18 nM (FIG. 18d). With the fluorescein conjugate, we also performed a competition experiment with nutlin-3a that binds to a defined hydrophobic pocked of MDM2 (B. Anil et al., Acta Crystallogr. Sect. D Biol. Crystallogr. 2013, 69, 1358-1366) and is a precursor of nutlin-based clinical candidates. The two ligands did not compete, indicating that the macrocycle binds to a different site and potentially has a new inhibition mechanism (FIG. 29). Repetition of the binding measurements of the macrocycles by surface plasmon resonance (SPR) as an orthogonal method showed Kds of 600±300 nM (M6), 550±190 nM (M7), 169±93 nM (M8), and 29±14 nM (M10), which confirmed the affinity range found with the FP assay (FIG. 18e and FIG. 30).

### 3.9 Conclusion

Herein a method for the diversification of macrocyclic backbones on picomolar scale using acoustic dispensing of activated carboxylic acids is described. Starting from SPPS, in just four days thousands of diverse macrocycles as crude reaction mixtures were obtained. These mixtures were then directly screened against protein targets, and inhibitors were successfully identified. The nanoliter volume acylation reaction was robust and resulted in high conversion to product across many different carboxylic acids and macrocycles. Unexpected byproducts were not observed. Our automated platform allowed for this final diversification step to be performed in under one hour. This is in contrast to the syntheses of traditional macrocycle libraries that require laborious purification. While acoustic dispensing has been used for synthesis in the past, it has been limited to the elaboration of synthetic reaction scopes, rather than for the direct synthesis of screening compounds. The small scale of our reactions, along with their relative purity, allowed for direct screening of macrocycles against protein targets in the same microtiter plate. It was demonstrated that inhibitors could be identified from these screens, and that the observed activity was due to the desired products rather than impurities. The screening of crude mixtures remains limited in the literature. The inventors are not aware of any existing efforts on the same scale, or specifically related to macrocycles. Given the facile nature of our method, it should be applicable to the screening of many protein targets. In addition, many different chemical reactions could be utilized for the ADE-based diversification; amide bond formation was chosen as an initial example due to its simplicity, and its previous use as a reaction for crude screening. Though the largest library synthesized by the inventors was 20,000 macrocycles, it could be expanded to hundreds of thousands with relative ease. To produce more lead-like compounds in the future, the method can be applied to backbone structures cyclized via non-reducible bonds. Building block sets could be expanded beyond amino acids in order to further increase the drug-like properties of the library. The method can be used to screen more therapeutically relevant targets for which it is desirable to develop clinical candidates.

### 3.10 Supplementary Results

### Overall structure of human α-thrombin in complex with M1

Human α-thrombin consists of two polypeptide chains of 36 (light chain) and 259 amino acid residues (heavy chain) covalently linked via a disulfide bridge (Cys122 of H-chain with Cys1 of L-chain). X-ray structure analysis of crystals formed by x-thrombin (light- and heavy-chain) and macrocycle M1 showed four nearly identical copies of heavy- and light-chains of human cx-thrombin in the asymmetric unit. The four light/heavy chains of xx-thrombin are named A/B, C/D, E/F, and H/L. The structure of H/L was used for all calculations and for preparing the structure figures. The light-chain of human α-thrombin can be traced unambiguously from Glu1C to Ile14K. The amino terminal residues (Thr1H to Gly1D) and the carboxyl-terminal residues Asp14L (except for light-chain C and E), Gly24M and Arg15 are undefined and not visible in the Fourier map. The electron density of the heavy-chain is clearly visible for all residues with the exception of few amino acids that are part of the surface flexible autolysis loop (Trp148 to Val149C). The carboxyl-terminal residue Glu247 lacks adequate electron density. Minor differences occur at the level of flexible and less defined loops or in the orientation of exposed peripheral side chains. The overall structure of human cx-thrombin bound to the macrocycle does not show any striking rearrangements of the main backbone if compared to other human α-thrombin structures, neither in the apo form, nor in complex with inhibitors.

### Overall Structure of Macrocycle M1

The electron density of the macrocycle M1 is well-defined allowing an unambiguous assignment of group orientations for all the four protein complexes present in the asymmetric unit. The numbering of the atoms in M1 is shown in FIG. 26b. No classical secondary structure elements and no non-covalent intra-molecular interactions are found in the macrocycle. The molecule appears to adopt a chair-like conformation that fits well the shape of the catalytic pocket.

### Interactions Between Human α-Thrombin and M1

The M1 macrocycle fits well into the cleft formed by the active site and the surrounding substrate pockets covering a protein surface of 400.5 Å² (N. Voss et al., Nucleic Acids Res. 2010, 38, W555-W562). The macrocycles' conformations and interactions are equivalent in the four active sites of the four-thrombin molecules present in the asymmetric unit. A large portion of interactions of M1 with human α-thrombin are mediated by the 5-chlorothiophene-2-carboxamide functional group that accommodates in the primary specificity S1 pocket. This group is trapped in the pocket by a hydrogen bond with the main chain of Gly219 (M1 N7 with Gly219 O) and a molecule of H2O that bridges the oxygen O9 of M1 with the main chain nitrogen of Glyl93 N and the main chain nitrogen of Ser195. 5-chlorothiophene-2-carboxamide is further involved in a network of polar contacts with the main chain of the nearby Cys191 (M1 09 with Cys191 O), Glu192 (M1 O9 with Glu192 N), Gly216 (M1 N7 with Gly216 O and M1 S15 with Gly216 N), Trp215 (M1 S15 with Trp215 N) and the side chain of Cys220 (M1 N7 with Cys220 S). The chlorine atom 5-chlorothiophene-2-carboxamide functional group points toward the bottom of the S1 pocket where it forms likely a halogen-aromatic π interaction (4.0 Å) with the aromatic ring of Tyr228. The main chain nitrogen N4 and oxygen O17 of M1 form hydrogen bonds with the main chain oxygen of Gly216 (Gly216 O) and nitrogen of Gly216 (Gly216 N), respectively. Additionally, the main chain nitrogen N4 and oxygen O17 of M1 can form polar contacts with the main chain nitrogen of Gly219 (Gly219 N) and oxygen of Gly216 (Gly216 O), respectively. Similarly, the main chain nitrogen N18 of M1 can form two polar contacts with the side chain carboxylic group of Glu192 (Glu192 OE1 and OE2). Finally, a molecule of H2O bridges the main chain nitrogen N27 of M1 with the main chain oxygen of Glu97A (Glu97A O). Importantly, the binding of M1 to human α-thrombin is mediated by multiple hydrophobic contacts by main and side chains of adjacent enzyme residues. The macrocycle backbone (C20-C24), including the disulfide bridge S21-S22, lays towards the hydrophobic cage shaped by the side chains of residues His57, Tyr60A, Trp60D (proximal S2 pocket) and Leu99 (distal S3 pocket). The valine side chain (C28-C31) bends the other side of the ring toward the hydrophobic pocket formed by Ile174 and Trp215. Finally, the C35-C40 phenyl ring run on top of a thrombin loop (Gly216-Cys220).

### 3.11 Supplementary Materials and Methods

### General Considerations

Unless otherwise noted, all reagents were purchased from commercial sources and used with no further purification. Solvents were not anhydrous, nor were they dried prior to use. The following abbreviations are used: DIPEA (N,N-diisopropylethylamine), DABCO (1,4-diazabicyclo[2.2.2]octane), NMM (4-methylmorpholine), HBTU (N,N,N′,N′-tetramethyl-O-(1H-benzotriazol-1-yl)-uronium-hexafluorophosphate), HATU (N,N,N′,N′-tetramethyl-O-(7-azabenzotriazol-1-yl)uronium-hexafluorphosphate), HEPES (4-(2-hydroxyethyl)piperazine-1-ethanesulfonic acid)

### Synthesis of Model Scaffold 1

The cyclic peptide model scaffold 1 was synthesized using the cyclative disulfide release strategy (CDR) previously described (S. Habeshian et al., ACS Chem. Biol. 2022, 17, 181-186). The linear peptide precursor was synthesized on a 25 μmol scale in a 5 ml polypropylene synthesis column (MultiSyntech GmbH, V051PE076) using Rapp Polymere HA40004.0 Polystyrene A SH resin (200-400 mesh), 0.95 mmol/gram loading resin and following the procedure described in Habeshian, S. et al. 2022². The peptide was released as follows. For deprotection of the side chains, the resin was incubated with 2 ml of 38:1:1 TFA/TIS/ddH₂O v/v/v for 1 hour and then washed with 5×4 ml of DCM. For cyclative peptide release, the resin was treated with 1 ml of DMSO containing 150 mM DIPEA (6 equiv.) overnight. The resin was removed by filtration. The crude mixture was purified by RP-HPLC using a Waters HPLC system (2489 UV detector, 2535 pump, Fraction Collector III), a 19 mm×250 mm Waters XTerra MS C18 OBD Prep Column C18 column (125 Å pore, 10 μm particle), solvent systems A (H₂O, 0.1% v/v TFA) and B (MeCN, 0.1% v/v TFA), and a gradient of 0-25% solvent B over 30 minutes. The fraction containing the model scaffold was lyophilized and dissolved in DMSO to reach a concentration of 40 mM.

### Acylation of Model Scaffold 1 by Pipetting of Reagents

The model scaffold was acylated at a 40 nmol scale in volumes of 4 μl as follows. The scaffold (20 μl of a 40 mM stock in DMSO) was supplemented with base (20 μl of 160 mM DIPEA dissolved in DMSO), and 2 μl of the mixture were transferred to wells of a PCR plate. The carboxylic acids were prepared as 160 mM stocks in DMSO containing 160 mM DIPEA. Equal volumes of HBTU (160 mM in DMSO) were added to each acid stock, and 2 μl of the resulting active esters (80 mM) were added to the same PCR plate. The reactions were allowed to proceed for 3 hours at room temperature. After this time, 1 μl of the reaction was transferred into 99 μl of 100 mM Tris-HCl in water pH 7.5, incubated for 6 hours to allow quenching of activated acids with Tris, and the reactions analyzed by LC-MS.

### Acylation of Model Scaffold 1 by Acoustic Reagent Transfer

The model scaffold was acylated at an 800 pmol scale in volumes of 80 nl as follows. Scaffold 1 (20 μl of a 40 mM stock in DMSO) was supplemented with base (20 μl of 160 mM DIPEA, 20 μl of 160 mM DABCO, 20 μl of 160 mM HEPES sodium salt or 20 μl of 1 M NMM dissolved in DMSO) and 10 μl of the mixtures were transferred to an ECHO source plate (Labcyte Echo Qualified 384-well Low dead volume microplate). The concentrations in the source plate were 20 mM model scaffold and 80 mM DIPEA (4 equiv.), or 80 mM DABCO (4 equiv.), or 800 mM NMM (40 equiv.). The carboxylic acids were prepared as 160 mM stocks in DMSO containing either 160 mM DIPEA, 160 mM DABCO, or 1 M NMM. An equal volume of HBTU (160 mM in DMSO) was added to each acid stock and the active esters (80 mM) were added to the same source plate. The source plate was centrifuged at 950 g (2,000 rpm with a Thermo Heraeus Multifuge 3L-R centrifuge) for 3 minutes to remove potential bubbles. Using a Labcyte Echo 650 acoustic dispenser, 40 nl of the model scaffold 1 (800 pmol) were transferred to a Nunc 384 well low volume polystyrene plate, followed by 40 nl of the active esters (3.2 nmol, 4 equiv.). The transfers were performed in duplicate in order to have enough material for LC-MS analysis. The plates were sealed, and the reactions were allowed to proceed for 6 hours at room temperature. After this time, 8 μl of 100 mM Tris-HCl in water pH 7.5 were added to each one of the duplicate reactions, the duplicates pooled, incubated for 3 hours to allow quenching of activated acids with Tris, and the reactions analyzed by LC-MS.

### Acylation of Model Scaffolds 2-5 by Acoustic Reagent Transfer

The model scaffolds 2-5 purchased from Enamine were obtained as 1.1 to 1.2 mg powders. The scaffolds were dissolved in 62 to 88 μl DMSO to obtain 40 mM stocks. The scaffolds were acylated using DABCO as a base, as described for the model scaffold 1 above, with the following differences: 6 hours reaction time. Before LC-MS analysis, 720 nl of DMSO was dispensed to each well, then 7.2 μl of 100 mM Tris-HCl in water pH 7.5 was dispensed. Quenching took place overnight.

### Design of Scaffolds and Amino Acid Sequences

The cyclic peptide scaffolds used for Library 1 were prepared by randomly choosing amino acid sequences. The number of different sequences that could theoretically be generated based on the chosen scaffold formats and amino acid building blocks was much larger than the number of scaffolds that were synthesized for Library 1 (384), as described in the following:

Theoretical number of scaffolds for Library 1:
Scaffolds containing di-amino acids: 3,240
   # scaffold formats (6)×# di-aa (6)×# backbone aa (6)×# side chain aa (15)
Scaffolds containing cysteine: 540
   # scaffold formats (6)×#backbone aa (6)×# side chain aa (15)

For randomly choosing 384 amino acid sequences, all building blocks were assigned an alphanumeric identifier, and every possible permutation was enumerated manually. The peptides were assigned numbers from 1 to 3,780. A random sequence generator (https://www.random.org/sequences/) was then used to re-order the numbers, and the first 384 were chosen for synthesis.

### Preparation of Polystyrene-S-S-Cysteamine Resin for Library Synthesis

The following procedure was applied to prepare polystyrene-S-S-cysteamine resin for the synthesis of 4×96 peptides at a 5 μmol scale in four 96-well plates, as needed for the synthesis of the scaffolds for Library 1 (thrombin screen). Into each of four 20 ml plastic syringes (CEM, 99.278) was added 589 mg resin (Rapp Polymere HA40004.0 Polystyrene A SH resin, 200-400 mesh), 0.85 mmol/gram loading, corresponding to a 0.5 mmol scale. The resin was washed with 15 mL of DCM, then swelled in 15 ml of 3:7 MeOH/DCM v/v for 20 minutes. 2-(2-pyridinyldithio)-ethanamine hydrochloride (1.96 grams, 8.8 mmoles, 4.4 equiv.) was dissolved in 21.12 ml of MeOH, then 49.28 ml of DCM and 1.53 ml of DIPEA were added. 17.7 ml of this solution was pulled into each syringe, which were then shaken at room temperature for 3 hours. After this time, the 2-(2-pyridinyldithio)-ethanamine solutions were discarded, and the resins were washed with 2×20 ml 3:7 MeOH/DCM v/v, then 2×20 ml DMF. The resins were combined into a single syringe as a suspension in DMF, then washed with 11.8 ml of 1.2 M DIPEA solution in DMF for 5 minutes to ensure that all amines were neutral. This solution was discarded, and the resin was washed with 2×20 ml DMF, 4×20 ml DCM, then kept under vacuum overnight to yield a free-flowing powder. For the synthesis of scaffolds for Library (MDM2 screen), the resin loading was 0.95 mmoles/gram, and thus 526 mg of resin was added to each of two syringes.

### Peptide Library Synthesis in 96-Well Plates

Automated solid-phase peptide synthesis was performed on an Intavis Multipep RSi synthesizer. For the thrombin library, to a 50 ml tube was added 565 mg of polystyrene-S-S-cysteamine resin (0.48 mmol cysteamine assuming that thiol groups were quantitatively modified with cysteamine) and 20 ml of DMF. For the MDM2 library, 505 mg of functionalized resin was added instead. The tube was shaken to ensure the resin was uniformly suspended, and 200 μl (5.88 mg resin, 5 μmoles) were transferred to each well of a 96-well solid phase synthesis plate (Orochem OF 1100). The resin was washed with 6×150 μl DMF. Coupling was performed with 53 μl of amino acids (500 mM, 5.3 equiv.), 50 μl HATU (500 mM, 5 equiv.), 12.5 μl of N-methylmorpholine (4 M, 10 equiv.), and 5 μl N-methylpyrrolidone. All components were premixed for 1 minute, then added to the resin (1 hour reaction, no shaking). The final volume of the coupling reaction was 120.5 μl and the final concentrations of reagents were 220 mM amino acid, 208 mM HATU and 415 N-methylmorpholine. Coupling was performed twice, then the resin was washed with 6×225 μl of DMF. Fmoc deprotection was performed using 120 μl of 1:5 piperidine/DMF v/v for 5 minutes, and was performed twice. The resin was washed with 8×225 μl DMF. At the end of the peptide synthesis, the resin was washed with 2×200 μl of DCM.

### Library Side Chain Protecting Group Removal

For side chain protecting group removal, the bottom of the 96-well synthesis plate was sealed by pressing the plate onto a soft 6 mm thick ethylene-vinyl acetate foam pad (Rayher Hobby GmbH, 78 263 01), and the resin in each well was incubated with around 500 μl of 38:1:1 TFA/TIS/ddH₂O v/v/v for 1 hour. The plates were covered with a polypropylene adhesive seal, then weighed down by placing a weight (1 kg) on top to ensure that no leakage occurred. After 1.5 hours, the synthesis plates were placed onto 2 ml deep-well plates (Thermo Scientific, 278752), and the TFA mixture was allowed to drain. The wells were washed with 3×500 μl of DCM (added with syringe), then allowed to air dry for 3 hours.

### Library Cyclative Release of Library Peptides in 96-Well Plates

Plates were pressed into foam pads as described above to plug the openings, and 200 μl of 150 mM DABCO in DMSO (6 equiv.) were added to each well. The plates were sealed with an adhesive foil and weighed down (1 kg), and left overnight. The next day, the synthesis plates were placed onto 2 ml deep-well plates (Thermo Scientific, 278752) and centrifuged at around 200 g (1,000 rpm with a Thermo Heraeus Multifuge 3L-R centrifuge) for 1 minute to collect the cleaved macrocycles in DMSO.

### Library Peptide Quantification by Absorption

Absorbance measurements were performed with a Nanodrop 8000 spectrophotometer (Thermo Scientific) at a wavelength of 280 nm using a 10 mm path length. Cleaved peptides containing Trp and D-Trp were diluted 250 fold into water for the thrombin library, and 125 fold into water for the MDM2 library. The Beer-Lambert law was used to calculate the concentration of the peptides. Extinctions coefficient Trp ε₂₈₀=5,500 M⁻¹cm⁻¹ was used.

### LC-MS Analysis

Peptides were analyzed by LC-MS analysis with a UHPLC and single quadrupole MS system (Shimadzu LCMS-2020) using a C18 reversed phase column (Phenomenex Kinetex 2.1 mm×50 mm C18 column, 100 Å pore, 2.6 μm particle) and a linear gradient of solvent B (acetonitrile, 0.05% formic acid) over solvent A (H₂O, 0.05% formic acid) at a flow rate of 1 ml/minute. Mass analysis was performed in positive ion mode.

For the LC-MS analysis, the samples of the various experiments were prepared as follows. For analyzing the acylation proof of concept reactions, 160 nl of reaction mixtures were diluted into 16 μl of Tris-HCl buffer pH 7.5 to give a peptide concentration of 100 μM. For analyzing the scaffolds synthesized for Library 1, 1 μl of the DMSO/DABCO eluates were diluted into 80 μl of water to give a cyclic peptide concentration of around 120 M. For analyzing the scaffolds synthesized for Library 2, 1 μl of the DMSO/DABCO eluates were diluted into 128 μl of water to give cyclic peptide concentration of around 120 μM. For all analyses, 5 μl of the samples were injected, typically using a 0 to 60% gradient of solvent B over 5 minutes.

### Calculation of Physicochemical Properties of Macrocycles

The physicochemical properties molecular weight, calculated water/n-octanol partition coefficient (cLogP), number of hydrogen bond donors (HBDs), number of hydrogen bond acceptors (HBAs), polar surface area (PSA), and number of rotatable bonds (NRotB) were calculated using DataWarrior software (www.openmolecules.org). The structures of the scaffolds and the carboxylic acids were drawn in ChemDraw and saved as SMILES strings in SD files, one for the scaffolds and one for the acids. Both SD files were opened in DataWarrior. The “enumerate combinatorial library” functionality was used to define the desired amide bond forming reaction between the macrocycle scaffolds and the carboxylic acids. The following definitions were made: amide was defined as “excluded group”. Nitrogen atom was defined as not being part of an aromatic ring, and having a hydrogen atom count greater than 0. The carbon atom next to the amine was defined as being not aromatic, and not containing pi electrons. The starting material and product atoms were then mapped. Following combinatorial enumeration, the desired properties were calculated from the structures.

### Acylation of Scaffolds to Generate Library 1

Cyclic peptide scaffolds, in the solvent used to release the peptides from resin (DMSO containing 150 mM DABCO), were transferred to a Labcyte Echo Qualified 384-well Low dead volume microplate (10 μl per well). The concentrations of the cyclic peptide scaffolds were around 8.1 mM in average. Carboxylic acids were dissolved to 160 mM in DMSO containing 160 mM DABCO. An equal volume of HBTU (160 mM in DMSO) was added to each acid stock. The active esters (80 mM) were added to another low-dead-volume source plate. The source plates were centrifuged at 850 g (2,000 rpm with a Thermo Heraeus Multifuge 3L-R centrifuge) for 3 minutes to remove potential bubbles. Using a Labcyte Echo 650 acoustic dispenser, 20 nl of the scaffolds (160 pmol) were transferred to 384 well low volume polystyrene plates (Nunc, 264705), followed by 20 nl of the active esters (1.6 nmol, 10 equiv.). The plates were sealed, and the reaction was allowed to proceed for 6 hours at room temperature. After this time, 5 μl of Tris buffer (100 mM Tris-Cl, pH 7.5, 150 mM NaCl, 10 mM MgCl₂, 1 mM CaCl₂, 0.1% w/v BSA, 0.01% v/v Triton-X100) was dispensed into each well using a BioTek MultiFlo microplate dispenser. The reactions were quenched overnight at room temperature.

### Thrombin Inhibition Screen

Thrombin inhibition by the macrocycles of the Library 1 was assessed by measuring residual activity of thrombin in presence of the cyclic peptides at 11 μM average final concentration. The assays were performed in 384-well plates using Tris buffer at pH 7.4 (100 mM Tris-Cl, 150 mM NaCl, 10 mM MgCl₂, 1 mM CaCl₂, 0.1% w/v BSA, 0.01% v/v Triton-X100, and 0.6% v/v DMSO) using thrombin at a final concentration of 2 nM and the fluorogenic substrate Z-Gly-Gly-Arg-AMC at a final concentration of 50 μM. Thrombin (5 μl, 6 nM) in the Tris-Cl buffer described above was added to each peptide using a BioTek MultiFlo microplate dispenser, and incubated for 10 minutes at room temperature. The fluorogenic substrate (5 μl, 150 μM) in the same buffer was added using the BioTek MultiFlo microplate dispenser, and the florescence intensity measured with a Tecan Infinite M200 Pro fluorescence plate reader (excitation at 360 nm, emission at 465 nm) at 25° C. for a period of 30 minutes with a read every 3 minutes. The slope of each activity measurement curve was calculated by Excel. For the negative controls (20 wells containing DMSO but no macrocycle), an average slope was calculated. The percent of thrombin inhibition was calculated by dividing the slopes and multiplying the results by 100.

### Acylation of Scaffolds to Generate Library 2

Cyclic peptides scaffolds, in the solvent used to release the peptides from resin (DMSO containing 150 mM DABCO), were transferred to a Labcyte Echo Qualified 384-well polypropylene microplate (40 μL per well). The concentrations of the cyclic peptide scaffolds were around 12.9 mM in average. Carboxylic acids were dissolved to 184 mM in a 184 mM DMSO solution of DABCO. An equal volume of HBTU (184 mM in DMSO) was added to each acid stock. The active esters (92 mM) were added to the same polypropylene source plate. The source plates were centrifuged at 950 g (2,000 rpm with a Thermo Heraeus Multifuge 3L-R centrifuge) for 3 minutes to remove potential bubbles. Using a Labcyte Echo 650 acoustic dispenser, 12.5 nl of macrocycles (161 pmol) were transferred to 384 well low volume polystyrene plates (Nunc, 264705), followed by 17.5 nl of the active esters (1.61 nmol, 10 equiv.). The plates were sealed, and the reaction was allowed to proceed for 6 hours at room temperature. After this time, 5 μl of Tris buffer was dispensed into each well using a Gyger Certus Flex liquid dispenser, and the reactions were quenched overnight at room temperature.

### MDM2 Binding Screen

MDM2 binding by cyclic peptides was assessed by measuring displacement of a fluorescent p53 peptide probe in presence of the cyclic peptides at 11 μM average final concentration. The assays were performed in 384-well plates using PBS buffer at pH 7.4 (100 mM Na₂HPO₄, 18 mM KH₂PO₄, 137 mM NaCl, 2.7 mM KCl, 0.01% v/v Tween-20, and 3% v/v DMSO), MDM2 at a final concentration of 1.2 μM, and the fluorescent p53 peptide probe (FP53, sequence =5(6)-FAM-GSGSSQETFSDLWKLLPEN) at a final concentration of 25 nM. Premixed MDM2 and FP53 (10 μl, 1.8 mM MDM2, 37.5 nM FP53) in the PBS buffer described above was added to each peptide using a Gyger Certus Flex liquid bulk dispenser, and incubated for 30 minutes in the dark at room temperature. One fluorescence anisotropy reading was taken with a Tecan Infinite F200 Pro fluorescence plate reader (excitation at 485 nm, emission at 535 nm) at 25° C.The percentage of probe displacement was calculated using to the following formula,

$$\% probe displacement = \frac{N - X}{N - P} \times 100$$

where N is the average anisotropy of the negative controls (no inhibition), X is the value obtained for each well, and P is the average anisotropy of only the probe.

### Identification of Active Species in Reactions from Hits

The macrocycles identified as hits in the thrombin screen were resynthesized at a 40 nmol scale by reacting 5 μl of 8 mM cyclic peptide scaffolds in DMSO containing 150 mM DABCO with 5 μl of 80 mM carboxylic acid, 80 mM HBTU and 80 mM DABCO for 5 hours at room temperature. Remaining activated ester was quenched by addition of 1.25 ml of Tris buffer (100 mM Tris-Cl, 150 mM NaCl, 10 mM MgCl₂, 1 mM CaCl₂) and incubation overnight. The next day, 240 μl of MeCN and 1 ml of water were added, and the reactions were run over a C18 column (7.8 mm×300 mm Waters NovaPak C-18 column, 60 Å pore, 6 μm particle) on a Thermo Dionex HPLC using solvent A (H₂O, 0.1% v/v TFA) and a 10-80% gradient of solvent B (MeCN, 0.1% v/v TFA) over 20 minutes, and fractions were collected every minute. Fractions were lyophilized, dissolved in 120 μl of 2% DMSO in water. The activities of products in the fractions were measured using the same assays as described above, but in 96-well plates. 50 μl of each fraction was transferred to a 96-well assay plate (Greiner, 655101), followed by 50 μl of thrombin (6 nM in buffer). After 10 minutes of incubation, 50 μl of fluorogenic thrombin substrate (Z-Gly-Gly-Arg-AMC, 150 μM in buffer, 1% DMSO) was added and the plates were read and the data processed as described above. Compounds in active fractions were identified by mass spectrometry.

For hits from the MDM2 screen, reactions were performed in the same way but at a 50 nmol scale, and purified with the same method but a 10-80% gradient of solvent B and over 22 minutes. Fractions were lyophilized and dissolved in 40 μl of DMSO, 160 μl of water was added, and 5 μl of each fraction was transferred to a 384-well plate (Nunc, 264705), and 15 μl of premixed MDM2/FP53 peptide were added (final concentrations: 1.2 μM MDM2, 50 nM FP53, 5% DMSO).

### Crystallization of Thrombin with M1

Human α-thrombin was purchased from Haematologic Technologies (Catalogue number: HCT-0020). Protein-stabilizing agent was removed using a PD-10 desalting column (GE Healthcare) equilibrated with 20 mM Tris-HCl, 200 mM NaCl, pH 8.0 and the same buffer as solvent. Buffer exchanged human α-thrombin was incubated with the macrocycle M1 at a molar ratio of 1:3 and subsequently concentrated to 7.5 mg/ml by using a 3,000 MWCO Vivaspin ultrafiltration device (Sartorius-Stedim Biotech GmbH). Further M1 macrocycle was added during the concentration to ensure that a 3-fold molar excess is preserved. Crystallization trials of the complex were carried out at 293 K in a 96-well 2-drop MRC plate (Hampton Research, CA, USA) using the sitting-drop vapor-diffusion method and the Morpheus and LMB crystallization screens (Molecular Dimensions Ltd, Suffolk, UK). Droplets of 600 nl volume (with a 1:1 protein:precipitant ratio) were set up using an Oryx 8 crystallization robot (Douglas Instruments Ltd, Berkshire, UK) and equilibrated against 80 μl reservoir solution. Best crystals were obtained by applying micro-seeding to fresh drops that had been allowed to equilibrate for 2-3 days using the following mixture as precipitant agent: 20 mM sodium formate, 20 mM ammonium acetate, 20 mM sodium citrate tribasic dihydrate, 20 mM potassium sodium tartrate tetrahydrate, 20 mM sodium oxamate, 100 mM MOPS/sodium HEPES pH 7.5, 12.5% w/v PEG 1000, 12.5% w/v PEG 3350, 12.5% v/v MPD.

### Crystallization, Data Collection and Structure Determination

For X-ray data collection, crystals were mounted on LithoLoops (Molecular Dimensions Ltd, Suffolk, UK) and flash-cooled in liquid nitrogen. X-ray diffraction data of human α-thrombin in complex with M1 were collected at the i04 beamline of Diamond Light Source Ltd (DLS, Oxfordshire, UK). The best crystals diffracted to 2.27 Å maximum resolution. Crystals belong to the P21 space group, with unit cell dimensions a=56.25 Å, b=100.57 Å, c=108.90 Å and α=90°, β=90.11°, γ=90°. The asymmetric unit contains four molecules, corresponding to a Matthews coefficient of 2.78 Å³/Da and a solvent content of about 48% of the crystal volume. Frames were indexed and integrated with software XIA2, merged and scaled with AIMLESS (CCP4i2 crystallographic package) (M. Winn et al., Acta Crystallogr. D 2011, 67, 235-242). The structure was solved by molecular replacement with software PHASER (A. McCoy et al., J. Appl. Crystallogr. 2007, 40, 658-674) using as a template the model 6GWE (S. Kale et al., Sci. Adv. 2019, 5, eaaw2851). Refinement was carried on using REFMAC (A. Vagin et al. Acta Crystallogr. D 2004, 60, 2184-2195) and PHENIX (P. Adams et al., Acta Crystallogr. D 2010, 66, 213-221). Since the first cycles of refinement, a wide electron density corresponding to the bound ligand was clearly visible in the electron density map. Building of the macrocycle was performed by Molview, restraint file generated and optimized by Phenix ELBOW. The macrocycle was fitted manually by graphic software COOT (P. Emsley et al., Acta Crystallogr. D 2010, 66, 486-501). The final model contains 9,098 protein atoms, 160 macrocycle atoms, 4 Na⁺ atoms, and 399 water molecules. The final crystallographic R factor reached 0.189 (R_{free} 0.243). Geometrical parameters of the model are as expected or better for this resolution. The solvent excluded volume and the corresponding buried surface were calculated using PISA software and a spherical probe of 1.4 Å radius. Intra-molecular and inter-molecular hydrogen bond interactions were analyzed by PROFUNC (R. Laskowski et al., Nucleic Acids Res. 2005, 33, W89-W93), LIGPLOT+ (R. Laskowski et al., J. Chem. Inf. Model. 2011, 51, 2778-2786), and PYMOL software.

### Acylation of Scaffolds to Generate Libraries 3 and 4

The cyclic peptide scaffolds required for Libraries 3 and 4 were synthesized as described for those used in Library 2. Due to the presence of many N-methylated amino acids which are more difficult to couple, 200 mM HOAt was applied together with HATU. The scaffolds were diluted to 2 mM in DMSO, and 15 nL were transferred using acoustic dispensing, followed by 15 nl DMSO containing carboxylic acids (40 mM, 20 equiv.), HBTU (40 mM) and DABCO (40 mM). After 6 hours reaction at room temperature, 370 nl of DMSO was added to each well, followed by 5 μl of 100 mM Tris-Cl pH 7.4 containing 0.01% v/v Tween 20, for quenching overnight. For the MDM2 binding screen, F-M8 was used as a fluorescent probe because of its higher affinity for MDM2, allowing to use the target protein at a lower concentration (720 nM, leading to around 55% bound probe). A volume of 35 μl of PBS buffer at pH 7.4 (100 mM Na₂HPO₄, 18 mM KH₂PO₄, 137 mM NaCl, 2.7 mM KCl, 0.01% v/v Tween-20 containing 28.6 nM F-M8 and 823 nM MDM2 were added to each well and the displacement of reporter probe determined as described above. The concentrations of the macrocycles were 750 nM.

### Synthesis of Macrocycles at mg Scale

Automated solid-phase peptide synthesis was performed on an Intavis Multipep RSi synthesizer. To a 5 ml syringe (MultiSyntech GmbH, V051PE076) was added 25 μmoles of polystyrene-S-S-cysteamine resin. The resin was washed with 6×150 μl DMF. Coupling was performed with 210 μl of amino acids (500 mM, 4.2 equiv.), 200 μl HATU (500 mM, 4 equiv.), 50 μl of N-methylmorpholine (4 M, 8 equiv.), and 5 μl N-methylpyrrolidone. All components were premixed for 1 minute, then added to the resin (1 hour reaction, no shaking). The final volume of the coupling reaction was 465 μl and the final concentrations of reagents were 226 mM amino acid, 215 mM HATU and 430 N-methylmorpholine. Coupling was performed twice, then the resin was washed with 2×600 μl of DMF. Fmoc deprotection was performed using 450 μl of 1:5 piperidine/DMF v/v for 5 minutes, and was performed twice. The resin was washed with 7×600 μl DMF. At the end of the peptide synthesis, the resin was washed with 2×600 μl of DCM.

After SPPS, the resin was incubated with 2 ml of 38:1:1 TFA/TIS/ddH2O v/v/v for 1 hour. The TFA solution was discarded, and the resin was washed with 5×4 ml DCM. After air drying for 3 hours, 1 ml of 150 mM DIPEA in DMSO was pulled in, and the syringes were shaken overnight at room temperature. The following day, the DMSO solutions were pushed into 50 ml conical tubes.

Carboxylic acids were typically coupled by adding 500 μl of premixed acids (100 mM, 2 equiv.), HBTU (100 mM) and DABCO (100 mM) in DMSO. After 3 hours at room temperature, 8 ml of water was added and the tubes were frozen, then lyophilized for two days to remove DMSO. The contents of the tubes were dissolved in 3 ml of MeCN, followed by addition of 7 ml of water.

The crude mixtures were purified by RP-HPLC using a Waters HPLC system (2489 UV detector, 2535 pump, Fraction Collector III), a 19 mm×250 mm Waters XTerra MS C18 OBD Prep Column (125 Å pore, 10 μm particle), solvent systems A (H₂O, 0.1% v/v TFA) and B (MeCN, 0.1% v/v TFA), and typically a gradient of 30-70% solvent B over 30 minutes.

Determination Kᵢs of Thrombin Inhibitors

Purified thrombin inhibitors (10 mM in DMSO) were diluted to 80 μM in 125 μl of Tris buffer (100 mM Tris-Cl, 150 mM NaCl, 10 mM MgCl₂, 1 mM CaCl₂) containing 0.1% w/v BSA, 0.01% v/v Triton-X100 and 0.2% DMSO. The macrocycles were diluted two-fold in Tris buffer containing 0.1% w/v BSA, 0.01% v/v Triton-X100 and 1% DMSO in buffer. The thrombin activity was measured in 96-well plates (Greiner, 655101) and the residual activity calculated as described above in the assay used to measure activities of HPLC-separated fractions of screening hits. The residual activity was plotted against the Log of the corresponding macrocycle concentrations, and sigmoidal curves were fitted using the following four-parameter equation in GraphPad Prism 6:

$$Y = Bottom + \frac{Top - Bottom}{( 1 + {10}^{( LogIC 50 - X ) * HillSlope} )}$$

Kᵢ values were determined from the IC₅₀ values using the Cheng-Prusoff equation (Kₘ=168 μM for thrombin and the applied substrate):

$${K}_{i} = \frac{{IC}_{50}}{1 + \frac{[ S ]}{{K}_{m}}}$$

### Determination of IC₅₀ of MDM2-Binding Macrocycles

The concentrations at which MDM2 macrocycles displaced the reporter peptide for 50% of the protein (IC₅₀) were determined with the above described fluorescence polarization competition assay. Volumes of 5 μL of purified macrocycles (20 mM in DMSO) were serial diluted two-fold in 100% DMSO in a low dead-volume ECHO source plate. Using acoustic droplet transfer, 150 nl of each dilution was transferred to a 384 well low volume polystyrene plate (Nunc, 264705). A volume of 15 μL of MDM2/FP53 probe premix (1.2 μM MDM2, 25 nM FP53 probe) in PBS buffer pH 7.4 (100 mM Na₂HPO₄, 18 mM KH₂PO₄, 137 mM NaCl, 2.7 mM KCl, 0.01% v/v Tween-20) containing 1% v/v DMSO was added to each well, and incubated for 30 minutes in the dark. Fluorescence anisotropy was measured as described above. The percentage of bound inhibitor was calculated using the following equation

$$\% bound inhibitor = \frac{N - X}{N - P} \times 100$$

where N is the average anisotropy of the DMSO controls, X is the anisotropy value obtained for each well, and P is the average anisotropy of the unbound probe. The ICsos were determined by plotting the percent of bound inhibitor against the logarithm of the corresponding macrocycle concentration, and the curves were fitted in GraphPad Prism 6 as described above.

### Synthesis of Fluorescein-Labeled Macrocycles

Fluorescein-labeled macrocycles were synthesized essentially as described in the mg-scale macrocycle synthesis procedure. For 5(6)-FAM, manual coupling was performed using 4 equiv. of acid (180 mM, 556 μl), 4 equiv. HATU (500 mM, 200 μl), 10 equiv. NMM (4 M, 62.5 μl), all in DMF. The coupling was performed 1×2 hours, then washed as previously described.

### Determining K_{d}s of Fluorescein-Labeled MDM2 Binders by FP

Fluorescein labeled macrocycle stocks (20 mM in DMSO) were diluted to a concentration of 10 μM by adding 0.5 μl into 999.5 μl of PBS. These dilutions were further diluted to a concentration of 100 nM by transferring 10 μl to 990 μl PBS, and 7.5 ul were transferred to wells of a 384 well low volume polystyrene plate (Nunc, 264705). Volumes of 7.5 μl of 2-fold dilutions of MDM2 in PBS were pipetted to the wells. The final concentrations of fluorescent macrocycles were 50 nM. After incubation of the plate for 30 minutes in the dark at room temperature, the fluorescence anisotropy was measured with a Tecan Infinite F200 Pro fluorescence plate reader (excitation at 485 nm, emission at 535 nm) at 25° C. Anisotropy was plotted against the logarithm of the corresponding MDM2 concentrations and sigmoidal curves were fitted as described above.

### Synthesis of Thrombin Inhibitors Containing Thioether Bonds

Linear peptides containing the three amino acids and the C-terminal cysteamine were synthesized by automated SPPS as described above for the synthesis of mg scale cyclic peptide, but at a 50 μmol scale and using cysteamine 4-methoxytrityl resin (Novabiochem 856087, 200-400 mesh, 1% DVB, 0.92 mmol/gram). To this peptide still on resin, 4-bromobutyric acid (500 μl, 500 mM, 10 equiv.) was coupled manually using N,N′-diisopropylcarbodiiimide (DIC, 500 μl, 500 mM, 10 equiv.) as an activating reagent and DMF as solvent. The acid and coupling reagent were premixed for 1 minute, then added to the resin (1 hour reaction with shaking). The final volume of the coupling reaction was 1 ml and the final concentrations of reagents were 250 mM amino acid, 250 mM DIC. Coupling was performed twice, then the resin was washed with 4×4 ml of DMF, then 2×4 ml DCM.

Side chain protecting group removal and cleavage was performed by incubating the resin with 2 ml of 38:1:1 TFA/TIS/ddH2O v/v/v for one hour with shaking. After this time, 50 ml of cold diethyl ether was added to the solution to precipitate the peptide. The mixture was stored at −20° C. for 30 minutes, then centrifuged for 30 minutes at 3,800 g (4,000 rpm on a Thermo Heraeus Multifuge 3L-R centrifuge) at 4° C. The ether was decanted, and the peptide pellet allowed to air dry for 15 minutes.

The peptide was dissolved in 50 ml of freshly de-gassed 1:4 water/acetonitrile and 200 μl (1.15 mmol, 23 equiv.) of neat DIPEA was added. The cyclization reaction was allowed to proceed at room temperature for 90 minutes, then frozen and lyophilized.

Carboxylic acid 14 was coupled as follows. The macrocycle was redissolved in 1 ml of DMSO containing 100 mM DABCO. Carboxylic acids were typically coupled by adding 500 μl of premixed acids (100 mM, 2 equiv.), HBTU (100 mM) and DABCO (100 mM) in DMSO. After 3 hours at room temperature, 8 ml of water was added and the tubes were frozen and lyophilized for 2 days to remove DMSO. The contents of the tubes were dissolved in 3 ml of MeCN followed by addition of 7 ml of water. The crude mixtures were purified by RP-HPLC as described above.

### Determining K_{d}s of MDM2 Binders by SPR

Experiments were performed using a GE Healthcare Biacore 8K instrument. MDM2 (10 μg/mL) was dissolved in 10 mM MES buffer (pH 6.0) and immobilized on three channels of a CM5 series S chip (Cytiva, 29104988) using EDC/NHS amine coupling conditions in running buffer (10 mM PBS pH 7.4, 150 mM NaCl, 3 mM KCI, and 0.005% v/v Tween-20). Typical immobilization level was 6,000 to 7,000 resonance units (RUs). The reference cell was treated the same way without MDM2. For the measurement of binding kinetics and dissociation constants, five serial dilutions (3-fold) of macrocycles plus a DMSO blank were prepared in running buffer (10 mM PBS PH 7.4, 150 mM NaCl, 3 mM KCl, and 0.005% v/v Tween-20, and 0.5% v/v DMSO) and analyzed in single cycle kinetics mode with contact and dissociation times of 120 seconds and 60 seconds, respectively.

### Example 4—Synthesis and Screening of a Large Macrocyclic Compound Library on the Edge of the Rule-of-Five

### 4.1. Results

In Example 1 1 (FIG. 14b; also named cysteamine) was conjugated onto thiol-functionalized resin via a dithiol exchange reaction with excess pyridyldithioethylamine. Here, activated thiosulfonates were used instead, as they could be synthesized without a chromatographic purification step and thus more easily in larger quantities. Towards this end, commercial vendors were searched for possible building blocks to afford N-Boc alkyl halogens, which could undergo a substitution reaction with sodium benzenethionosulfonate to afford Boc protected precursors in excellent yield in gram-scale (87%-quant., see the materials and methods section for synthesis). Despite limited commercial availability of amino-halogens, six new diversification building blocks where synthesized without the need for silica column purification. These could be taken further and be directly loaded onto high-loaded SH PS resin to afford 2-7 immobilized by a dithiol bridge on resin (FIG. 15a).

To test whether the building blocks were compatible with automated Fmoc-based SPPS, the model dithiol peptide was synthesized: MPA-Trp-Ala-(1-7) (MPA=3-mercaptopropionic acid) in a 96-well plate format (FIG. 15b). After synthesis and removal of protecting groups by TFA-treatment, the resin-linked peptides were incubated with 2×200 μl reductive release cleavage cocktail (1,4-butanedithiol (BDT) and NEt3, both 100 mM in DMF). The peptides were liberated from the resin and analyzed by HPLC-MS after removal of DMF and the volatile BDT by rotary vacuum concentration (RVC). The peptides were efficiently released and analyzed by LC-MS. For all peptides, the desired product was found in excellent quality (FIG. 15c).

With the resin-linked diversification elements in hand, these fragments were exploited for the synthesis of a macrocycle library to pursue binders against trypsin-like serine proteases involved in blood-coagulation pathways. Due to the shear amount of trypsin-like serine proteases and their roles in various diseases, these proteases serve as important target towards the development of potent, and specific inhibitors against this class of proteases. The complexity of this task arises from the structural identity shared in many members of this group, due to their ability to recognize and cleave positively charged residues, and a conserved aspartate buried deep in their S1 pocket. It was therefore hoped to use this model system for the development of potent, selective macrocyclic inhibitions as the results can later be translated into other parts of cyclic peptide research.

Therefore 384 dithiol peptides were prepared in 5 μmol scale by automated SPPS in 96-well filter plates. Peptides were synthesized in three different scaffolds (1a-c, FIG. 16a) containing randomly chosen sequences consisting of one of the seven different diversification elements (1-7), a random amino acid (from 27 different α, β, γ, and N-methylated amino acids) and an S1 pocket binding motif known from literature.

The peptides were synthesized on solid support, and concomitant protection groups were removed by acidic treatment with TFA. Subsequently, the peptides were liberated from the resin by reductive release affording the linear dithiol peptides. Upon acidification and removal of volatile reducing agent by RVC, the concentration of the peptides were determined using Ellman's reagent (average conc.=24.9 mM, 20% average from resin loading with an equal distribution of peptide will all 7 different derivatives).

Our latest approaches in the lab emphasized the library preparation in picomole scale for direct usage in assay-ready microtiter plates. The preparation of compound libraries was envisioned such that they could be used for screening several targets at once, and therefore necessitated an approach that still allowed for large compound diversification with several linkers, while still allowing for large compound preparation without the need for cumbersome and resource-intensive pipette transfers. The attention was therefore turned to the use of acoustic droplet ejection (ADE) technology dispensing. Even though the technique is primarily utilized for transfers in nl volumes, it has previously shown that even larger μl volumes can be transferred using the system, thus avoiding pipetting. Therefore 40 nmol of linear peptide were distributed into several Echo® ADE-compatible 384 polypropylene (PP) plates (FIG. 17). As the reduced dithiol-peptides was slowly subjected to oxidation upon dissolvation in DMSO, the peptides were quickly re-reduced by adding DMF: BDT solution to the wells, which after acidification and RVC was immediately cyclized in MeCN/NH4HCO3 buffer with the 7 diverse linkers (see FIG. 16B for selection). This is in contrast to previously employed methods where the linear species were first solubilized, followed by addition of linker solution. The new methodology minimizes the amount of re-oxidation that occurs, resulting in more pure reaction mixtures. Finally, excess linkers were quenched with B-mercaptoethanol (β-ME).

### 4.2. Material and Methods

All reagents and solvents were of analytical grade and used without further purification as obtained from commercial suppliers. Reactions were monitored by thin-layer chromatography (TLC) using silica gel coated plates (analytical SiO₂-60, F-254) and/or by HPLC-MS analysis. TLC plates were visualized under UV light or by dipping into a solution of potassium permanganate (10 g/L) followed by visualization with a heatgun. Rotary evaporation of solvents was carried out under reduced pressure at a temperature below 40° C. HPLC-MS analyses were performed with a UHPLC and single quadrupole MS system (Shimadzu LCMS-2020) using a C18 reversed phase column (Phenomenex Kinetex 2.1×50 mm C18 column, 100 Å pore, 2.6 um particle). A linear gradient of solvent B (0.05% HCOOH in MeCN) over solvent A (0.05% HCOOH in water) rising linearly from 0% to 60% during t=1.00-6.00 min was applied at a flow rate of 1.00 ml/min. Nuclear magnetic resonance (NMR) spectra were recorded on a Bruker Avance III (¹H NMR and ¹³C NMR recorded at 400 and 101 MHZ, respectively) equipped with a cryogenically cooled probe. All spectra were recorded at 298 K. Chemical shifts are reported in ppm relative to deuterated solvent as internal standard (δ_{H} DMSO-d₆ 2.50 ppm; δ_{C} DMSO 39.52 ppm; δ_{H} CDCl₃ 7.26 ppm; δ_{C} CDCl₃ 77.16 ppm).

High-resolution mass spectrometry (HRMS) measurements were recorded on a maXis G3 quadrupole time-of-flight (TOF) mass spectrometer (Bruker Daltonics, Bremen, Germany) equipped with an electrospray ionization (ESI) source.

### Chemical Synthesis Of Building Blocks and Amino Acids

### S-(3-((tert-butoxycarbonyl)amino)propyl) benzenesulfonothioate (S1, ALN-1-31)

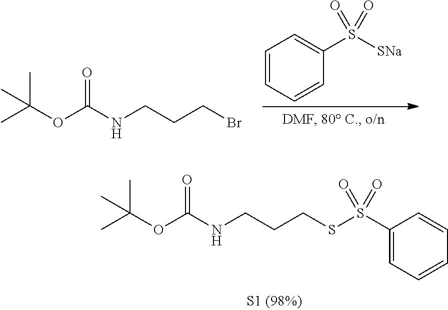

To a stirring solution of 3-(Boc-amino)propyl bromide (6.02 g, 25.3 mmol, 1.0 equiv.) in DMF (120 ml) was added sodium benzyl thiosulfonate (7.48 g, 38.0 mmol, 1.5 equiv.; tech. 85%) and the solution was stirred overnight at 80° C. After cooling down, the reaction mixture was concentrated under reduced pressure, resuspended in water (100 ml) and extracted with EtOAc:hexanes (2×150 ml, 10:1, v/v). The combined organic layers were washed with water (3×150 ml) and brine (150 ml), dried over anhydrous Na₂SO₄ and concentrated under reduced pressure to afford crude S1 (8.21 g, 24.8 mmol, 98%) as a yellow-tainted oil. TLC (25% EtOAc in hexanes): R_{f}=0.25 (KMnO₄ stain). ¹H NMR (400 MHZ, CDCl₃) δ7.98-7.90 (m, 2H), 7.70-7.61 (m, 1H), 7.60-7.52 (m, 2H), 3.16 (q, J=6.4 Hz, 2H), 3.02 (t, J=7.2 Hz, 2H), 1.83 (p, J=6.8 Hz, 2H), 1.43 (s, 9H).

### tert-butyl (3-chloropropyl)(methyl)carbamate (S2, VC-1-1)

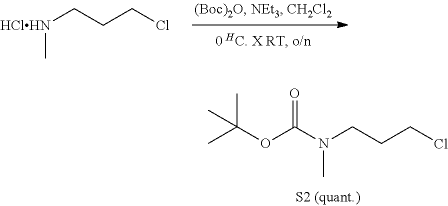

A stirring solution of 3-chloropropyl-N-methylamine hydrochloride (4.32 g, 30.0 mmol, 1.0 equiv.) and di-tert-butyldicarbonate (6.58 g, 30.0 mmol, 1.0 equiv.) in CH₂Cl₂ (150 ml) was cooled to 0° C. under argon atmosphere. NEt₃ (4.16 ml, 30.0 mmol, 1.0 equiv.) was added dropwise over 5 min and the solution was stirred overnight going towards ambient temperature. The reaction mixture was concentrated under reduced pressure and resuspended in EtOAc (120 ml). The solution was washed with aq. HCl (1 M, 2×120 ml), sat. NaHCO3 (120 ml) and brine (120 ml), dried over anhydrous Na₂SO₄ and concentrated under reduced pressure to afford crude S2 (6.22 g, 30.0 mmol, quant.) as a colorless crystalline solid. TLC (25% EtOAc in hexanes): R_{f}=0.45 (KMnO₄ stain). ¹H NMR (400 MHZ, CDCl₃) δ3.55 (t, J=6.5 Hz, 2H), 3.36 (t, J=6.8 Hz, 2H), 2.87 (s, 3H), 2.06-1.91 (m, 2H), 1.46 (s, 9H). CAS RN: 114326-14-6.

### S-(3-((tert-butoxycarbonyl)(methyl)amino)propyl) benzenesulfonothioate (S3, VC-1-3)

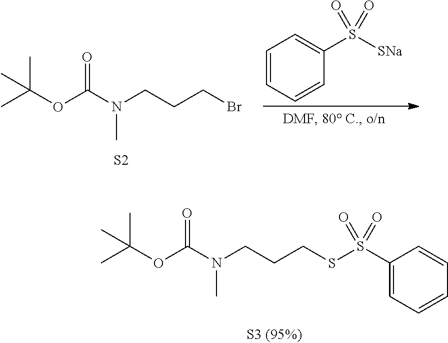

To a stirring solution of VC-1-1 (3.51 g, 16.9 mmol) in DMF (25 ml) was added sodium benzyl thiosulfonate (5.50 g, 27.9 mmol, 1.65 equiv.; tech. 85%) and the solution was stirred overnight at 80° C. After cooling down, the reaction mixture was concentrated under reduced pressure, resuspended in water (100 ml) and extracted with EtOAc:hexanes (2×75 ml, 10:1, v/v). The combined organic layers were washed with water (3×75 ml) and brine (75 ml), dried over anhydrous Na₂SO₄ and concentrated under reduced pressure to afford crude S3 (5.07 g, 16.2 mmol, 95%) as a yellow-tainted oil. TLC (12.5% EtOAc in hexanes): R_{f}=0.30 (KMnO₄ stain). ¹H NMR (400 MHZ, CDCl₃) 0 8.01-7.88 (m, 2H), 7.69-7.50 (m, 3H), 3.24 (t, J=6.7 Hz, 2H), 2.96 (t, J=7.3 Hz, 2H), 2.77 (s, 3H), 1.85 (p, J=6.8 Hz, 2H), 1.42 (s, 9H).

### tert-butyl (Z)-(4-chlorobut-2-en-1-yl)carbamate (S4, P1_N2_E29/E46)

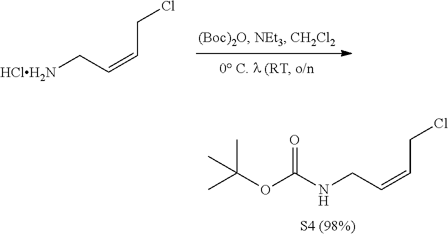

A stirring solution of cis-4-chloro-2-butenylamine hydrochloride (2.50 g, 17.6 mmol, 1.0 equiv.) and di-tert-butyldicarbonate (3.84 g, 17.6 mmol, 1.0 equiv.) in CH₂Cl₂ (75 mL) was cooled to 0° C. under argon atmosphere. NEt₃ (2.45 ml, 17.6 mmol, 1.0 equiv.) was added dropwise over 5 min and the solution was stirred overnight going towards ambient temperature. The reaction mixture was concentrated under reduced pressure and resuspended in CH₂Cl₂ (100 ml). The solution was washed with aq. HCl (1 M, 2×100 ml), sat. NaHCO₃ (100 ml) and brine (100 ml), dried over anhydrous Na₂SO₄ and concentrated under reduced pressure to afford crude S4 (3.55 g, 17.3 mmol, 98%) as a light-brown solid. TLC (25% EtOAc in hexanes): R_{f}=0.30 (KMnO₄ stain). ¹H NMR (400 MHZ, CDCl₃) δ5.82-5.70 (m, 1H), 5.70-5.56 (m, 1H), 4.59 (br s, 1H), 4.12 (d, J=7.8 Hz, 2H), 3.83 (t, J=6.6 Hz, 2H), 1.44 (s, 9H). CAS RN: 123642-28-4. NMR spectrum in agreement with literature.

### (Z)-S-(4-((tert-butoxycarbonyl)amino)but-2-en-1-yl) benzenesulfonothioate (S5, P1_N2_E30)

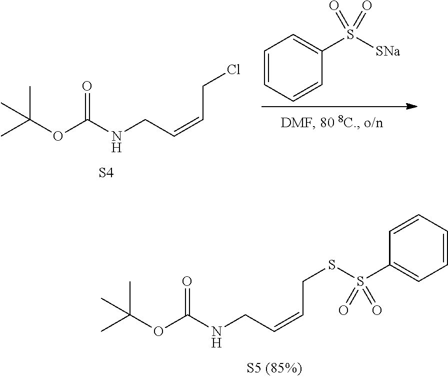

To a stirring solution of S4 (3.55 g, 17.3 mmol, 1.0 equiv.) in DMF (70 ml) was added sodium benzyl thiosulfonate (6.81 g, 34.5 mmol, 2.0 equiv.; tech. 85%) and the solution was stirred overnight at 80° C. After cooling down, the reaction mixture was concentrated under reduced pressure, resuspended in water (400 mL) and extracted with EtOAc (3×100 ml). The combined organic layers were washed with water (3×200 mL) and brine (200 mL), dried over anhydrous Na₂SO₄ and concentrated under reduced pressure to obtain crude S5 (5.03 g, 14.6 mmol, 85%*) as a brown oil. TLC (25% EtOAc in hexanes): R_{f}=0.20 (KMnO₄ stain). ¹H NMR (400 MHZ, CDCl₃) δ7.94-7.87 (m, 2H), 7.68-7.60 (m, 1H), 7.60-7.49 (m, 2H), 5.67-5.57 (m, 1H), 5.51-5.40 (m, 1H), 4.43 (br s, 1H), 3.67 (dq, J=7.0, 1.1 Hz, 2H), 3.60 (t, J=6.1 Hz, 2H), 1.43 (s, 9H) .* The crude compound purity is lower than for other thiosulfonate building blocks, but still provides excellent resin quality in the subsequent resin loading steps.

### tert-butyl 3-(((phenylsulfonyl)thio)methyl)azetidine-1-carboxylate (S6, ALN-1-57)

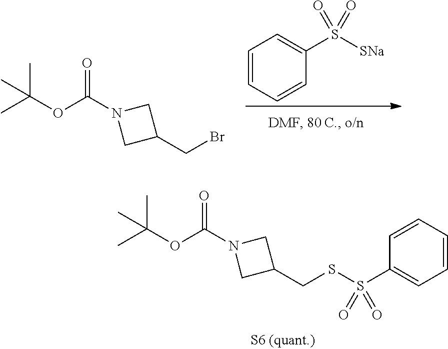

To a stirring solution of 1-Boc-3-bromomethylazetidine (1.93 g, 7.71 mmol, 1.0 equiv.) in DMF (25 ml) was added sodium benzyl thiosulfonate (2.43 g, 12.3 mmol, 1.6 equiv.; tech. 85%) and the solution was stirred overnight at 80° C. After cooling down, the reaction mixture was concentrated under reduced pressure, resuspended in water (100 ml) and extracted with EtOAc:hexanes (2×75 ml; 10:1, v/v). The combined organic layers were washed with water (2×75 ml), sat. NaHCO₃ (75 ml) and brine (75 ml), dried over anhydrous Na₂SO₄ and concentrated under reduced pressure to obtain crude S6 (2.65 g, 7.71 mmol, quant.) as a yellow-tainted oil. TLC (33% EtOAc in hexanes): R_{f}=0.30 (KMnO₄ stain). ¹H NMR (400 MHZ, CDCl₃) δ7.93 (d, J=7.5 Hz, 2H), 7.66 (t, J=7.4 Hz, 1H), 7.58 (t, J=7.6 Hz, 2H), 3.96 (t, J=8.6 Hz, 2H), 3.52 (dd, J=9.0, 5.3 Hz, 2H), 3.23 (d, J=7.8 Hz, 2H), 2.85-2.70 (m, 1H), 1.41 (s, 9H).

### tert-butyl 4-((phenylsulfonyl)thio)piperidine-1-carboxylate (S7, ALN-1-41)

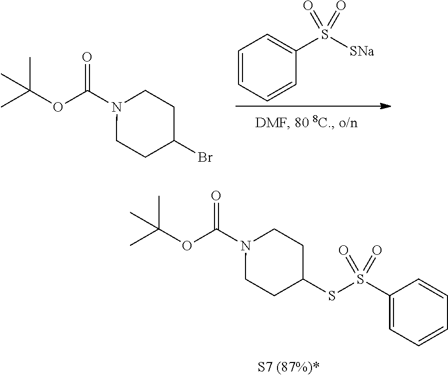

To a stirring solution of 1-N-Boc-4-bromopiperidine (2.25 g, 8.51 mmol, 1.0 equiv.) in DMF (25 mL) was added sodium benzyl thiosulfonate (2.77 g, 14.0 mmol, 1.65 equiv.; tech. 85%) and the solution was stirred overnight at 80° C. Due to incomplete overnight reaction (monitored by TLC), additional sodium benzyl thiosulfonate (1.38 g, 7.00 mmol; tech. 85%) was added and the reaction was stirred another 24 h at 80° C. After cooling down, the reaction mixture was concentrated under reduced pressure, resuspended in water (100 mL) and extracted with EtOAc:hexanes (2×75 ml; 10:1, v/v). The combined organic layers were washed with water (2×75 ml), sat. NaHCO₃ (75 ml) and brine (75 ml), dried over anhydrous Na₂SO₄ and concentrated under reduced pressure to obtain crude S7 (2.65 g, 7.43 mmol, 87%*) as a yellow-tainted oil. TLC (33% EtOAc in hexanes): R_{f}=0.44 (KMnO₄ stain). ¹H NMR (400 MHZ, CDCl₃) δ8.01-7.89 (m, 2H), 7.71-7.61 (m, 1H), 7.60-7.50 (m, 2H), 3.89-3.63 (m, 2H), 3.55-3.39 (m, 1H), 3.11-2.91 (m, 2H), 1.96-1.86 (m, 2H), 1.64-1.51 (m, 2H), 1.42 (s, 9H). * The substitution reaction progresses significantly slower than for the preparation of the other thiosulfonate building blocks. Additionally, the crude purity is lower, but still provides excellent resin quality in the subsequent resin loading steps.

### tert-butyl 4-(((phenylsulfonyl)thio)methyl)piperidine-1-carboxylate (S8, ALN-1-38)

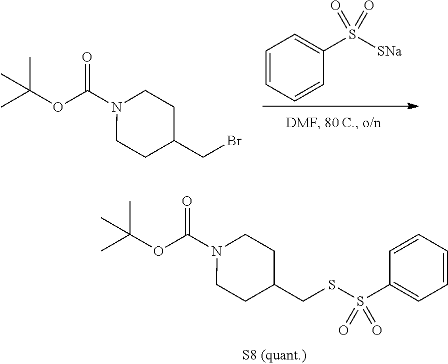

To a stirring solution of 1-N-Boc-4-(bromomethyl)piperidine (2.16 g, 7.76 mmol, 1.0 equiv.) in DMF (50 ml) was added sodium benzyl thiosulfonate (2.52 g, 12.8 mmol, 1.65 equiv.; tech. 85%) and the solution was stirred overnight at 80° C. After cooling down, the reaction mixture was concentrated under reduced pressure, resuspended in water (100 ml) and extracted with EtOAc:hexanes (2×75 ml; 10:1, v/v). The combined organic layers were washed with water (2×75 ml), sat. NaHCO₃ (75 ml) and brine (75 ml), dried over anhydrous Na₂SO₄ and concentrated under reduced pressure to obtain crude S8 (2.76 g, 7.76 mmol, quant.) as a clear oil. TLC (25% EtOAc in hexanes): R_{f}=0.30 (KMnO₄ stain). ¹H NMR (400 MHZ, CDCl₃) δ8.01-7.87 (m, 2H), 7.70-7.61 (m, 1H), 7.61-7.51 (m, 2H), 4.20-3.94 (m, 2H), 2.91 (d, J=6.5 Hz, 2H), 2.58 (t, J=12.8 Hz, 2H), 1.73-1.57 (m, 3H), 1.43 (s, 9H), 1.14-0.98 (m, 2H).

### (S)-2-((((9H-fluoren-9-yl)methoxy)carbonyl)amino)-3-(5-chlorofuran-2-carboxamido) propanoic acid (Thio, ALN-1-77).

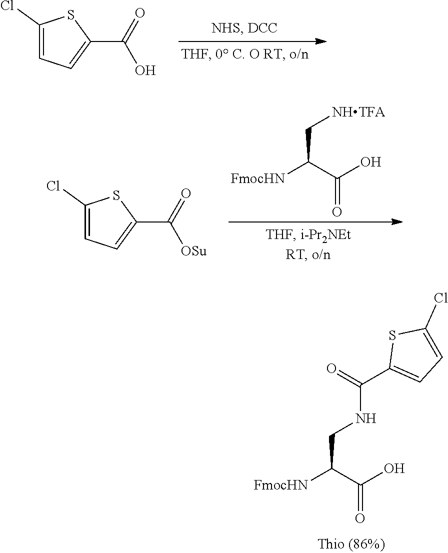

The synthesis was adapted from a previous procedure of similar amino acids: 5-chlorothiophene-2-carboxylic acid (2.44 g, 15.0 mmol, 1.5 equiv.) and N-hydroxysuccinimide (1.61 g, 14.0 mmol, 1.4 equiv.) were dissolved in THF (100 ml) and stirred under argon atmosphere. The solution was cooled to 0° C.after which a solution of DCC (2.89 g, 14.0 mmol, 1.4 equiv.) dissolved in THF (30 ml) was added slowly to the reaction mixture. The solution slowly became turbid and was allowed to stir overnight going towards ambient temperature, after which the solution was filtered. Meanwhile, Fmoc-Dap(Boc)-OH (4.26 g, 10.0 mmol, 1.0 equiv.) was stirred in CH₂Cl₂ (20 ml; turbid solution) and TFA (20 ml) was added slowly to the solution. The solution immediately became yellowish clear and bubbles where forming. After bubbling had stopped the solution was stirred an additional 30 min at ambient temperature after which solvent was removed under a stream of nitrogen. Excess TFA was removed by co-evaporation with CH₂Cl₂:toluene (50 ml, 1:1, v/v). The residue was resuspended in THF (40 ml) followed by addition of i-Pr₂NEt (5.75 ml, 60.0 mmol, 6.0 equiv.). The solution was then poured into the mother liquour containing the NHS-activated thiophene and stirred overnight at ambient temperature. After completion, the solution was concentrated under reduced pressure, redissolved in EtOAc:hexane (300 ml, 10:1, v/v) and washed twice with water (100 ml) and brine (100 ml). The organic layer was dried over anhydrous Na₂SO₄ and concentrated under reduced pressure. The crude product was purified by the silica column chromatography to afford the Fmoc-protected amino acid building block (4.06 g, 8.62 mmol, 86%) as an off-white solid*. TLC (5% MeOH and 0.5% AcOH in CH₂Cl₂): R_{f}=0.2 (UV). ¹H NMR (400 MHZ, DMSO-d₆) δ12.74 (br s, 1H), 8.68 (t, J=5.8 Hz, 1H), 7.89 (d, J=7.5 Hz, 2H), 7.70 (d, J=7.4 Hz, 2H), 7.65 (d, J=8.2 Hz, 1H), 7.61 (d, J=4.1 Hz, 1H), 7.41 (t, J=7.4 Hz, 2H), 7.30 (q, J=7.6 Hz, 2H), 7.18 (d, J=4.0 Hz, 1H), 4.43-4.11 (m, 4H), 3.68-3.50 (m, 2H, overlap with residual water). ¹³C NMR (101 MHZ, DMSO) δ171.9, 160.5, 156.0, 143.80, 143.77, 140.7, 138.7, 133.1, 128.2, 128.1, 127.6, 127.1, 125.24, 125.21, 120.1, 65.7, 53.5, 46.6, 40.3 (overlap with solvent peak). HRMS m/z calcd for C₂₃H₂₀ClN₂O₅l S⁺ [M+H]⁺, 471.0776; found 471.0786.

### Synthesis of Dithiol Resins

### Preparation of Highly Loaded Thiol-Resin

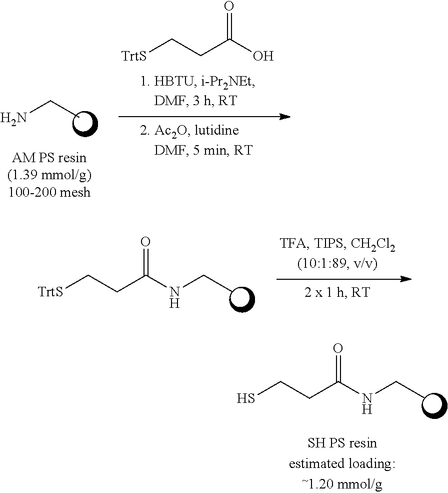

Pre-washing: Each 25 ml-fritted syringe was loaded with ˜0.8 g (1.11 mmol) aminomethyl polystyrene resin (AM PS resin; 1.39 mmol/g, 100-200 mesh; Aapptec, cat. #RAZ001) and pre-washed using MeOH (2×10 ml), CH₂Cl₂ (3×10 ml), 1% (v/v) TFA in CH₂Cl₂ (2×10 ml), i-Pr₂NEt in CH₂Cl₂ (1.2 M; 2×10 ml for 5 min), CH₂Cl₂ (2×10 ml) and DMF (2×10 ml). Coupling: A solution of 3-(tritylthio)propionic acid (1.16 g, 3.33 mmol, 3.0 equiv.) and HBTU (1.27 g, 3.33 mmol, 3.0 equiv.) in DMF (10 ml) was activated with i-Pr₂NEt (1.10 ml, 6.66 mmol, 6.0 equiv.) and added to the fritted syringe and agitated for 3 h at ambient temperature. The resin was filtered and washed with DMF (3×10 ml) and CH₂Cl₂ (3×10 ml) followed by drying the beads (first under suction and then under reduced pressure overnight (<0.5 mbar)). The loading of the MPA(Trt) resin was determined to ˜1.20 mmol/g* (weight based). Capping: A solution of 5% AC₂O and 6% lutidine in DMF (12 ml; v/v/v) was added to the resin and incubated it for 5 min at ambient temperature. The resin was drained and washed with DMF (3×10 ml) and CH₂Cl₂ (3×10 ml) Deprotection: A solution of 10% TFA and 1% TIPS in CH₂Cl₂ (15 ml, v/v/v) was added to the resin and agitated for 1 h at ambient temperature. The resin was washed with CH₂Cl₂ (3×10 ml) and the procedure was repeated once to afford high-loaded thiol resin on polystyrene (SH PS resin) that could be utilized for subsequent disulfide exchange and loading of cysteamine derivatives.

### Preparation of Resin 1 (res 1)

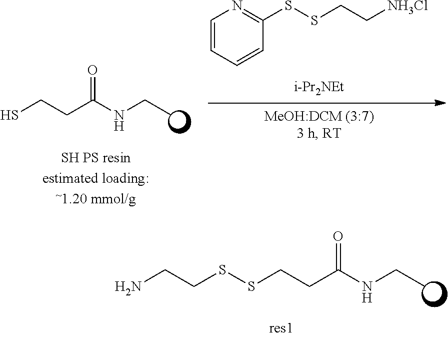

Dithiol exchange: Each 25 ml-fritted syringe was loaded with ˜0.4 g (0.48 mmol) SH PS resin was swelled in CH₂Cl₂ (10 ml) and then drained. 2-pyridylthio cysteamine hydrochloride salt (0.48 g, 0.96 mmol, 2.0 equiv.) was dissolved in MeOH: CH₂Cl₂ (19 ml, 3:7, v/v) followed by addition of i-Pr₂NEt (167 μl, 0.96 mmol, 2.0 equiv.). The solution was added to the resin and agitated for 3 h at ambient temperature. The resin was drained and washed with MeOH:CH₂Cl₂ (2×10 ml, 3:7, v/v), DMF (2×10 ml), i-Pr₂NEt in DMF (1.2 M; 10 ml for 5 min), DMF (3×10 ml) and CH₂Cl₂ (2×10 ml) followed by drying the beads (first under suction and then under reduced pressure overnight (<0.5 mbar)). Qualitative controls: (1) Kaiser test; complete purple/blue coloration of the beads. (2) Ellman's reagent on beads; no coloration.

### Preparation of Resins 2-7 (res 2-7)

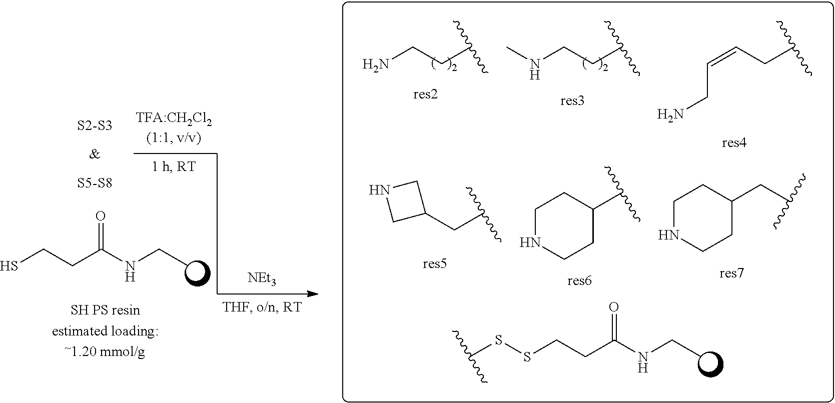

Deprotection: N-Boc protected thiosulfonate intermediate (S2, S3, S5, S6, S7 or S8; ˜ 7.0 mmol) was dissolved in CH₂Cl₂ (10 ml) followed by dropwise addition of TFA until heavy CO2 bubbling was observed (˜10 ml). The solution was stirred for another 1 h at ambient temperature whereafter solvent was removed under a stream of nitrogen. Excess TFA was removed under reduced pressure by co-evaporation with CH₂Cl₂:MeOH solution (1:1, v/v) to afford thiosulfonate salt for installation on resins. Dithiol exchange: Each 25 ml-fritted syringe was loaded with ˜0.8 g (0.96 mmol) SH PS resin was swelled in THF (15 ml) and then drained. The desired thiosulfonate TFA salt (2.40-2.88 mmol, 2.5-3.0 equiv.) was dissolved in THF (15 ml) and NEt₃ (803 μl, 5.76 mmol, 6.0 equiv.) was added. The solution was added to the resin and agitated overnight at ambient temperature. The resin was drained, washed with THF (3×15 ml) and CH₂Cl₂ (2×15 ml) followed by drying of the beads (first under suction and then under reduced pressure overnight (<0.5 mbar)). Qualitative controls: (1) Kaiser test; complete purple/blue coloration of the beads. (2) Ellman's reagent on beads; no coloration.

### Method Using Plates

Polypropylene (PP) 96-well filter plates were equipped with 5 μmol/well of polystyrene dithiol resins (res1-res7; estimated loading ˜1.20 mmol/g), and washed with DMF (6×225 μl). Coupling was performed with 53 μl of amino acids (500 mM, 5.3 equiv.), 50 μl HATU (500 mM, 5.0 equiv.), 13 μl of N-methylmorpholine (4 M, 10 equiv.), and 5 μl N-methylpyrrolidone. For couplings of Thio, t-acha, 4 amPip and 2 am, 75 μl of amino acids (170 mM, 2.55 equiv.), 25 μl HATU (500 mM, 2.5 equiv.), 7 μl of N-methylmorpholine (4 M, 5.6 equiv.), and 5 μl N-methylpyrrolidone were used. All components were premixed for one minute, then added to the resin (two hour reaction, no shaking). Coupling was performed twice and resin was washed with DMF (6×225 μl). Fmoc deprotection was performed using 20% (v/v) piperidine in DMF (120 μl, 2×2 min) and the resin was washed with DMF (6×225 μl). At the end of the peptide synthesis, the resin was washed with CH₂Cl₂ (2×200 μl) and resin beads were dried under suction.

### Method Using Syringes

To 5 ml syringe reactors was added polystyrene dithiol resins (res1-res7; estimated loading ˜1.20 mmol/g), and the resin was washed with DMF (6×150 μl). Coupling was performed with 210 μL of amino acids (500 mM, 4.2 equiv.), 200 μl HATU (500 mM, 4.0 equiv.), 50 μl of N-methylmorpholine (4.0 M, 8.0 equiv.) and 5 μl N-methylpyrrolidone. All components were premixed for one minute, then added to the resin (two hour reaction, with shaking). Coupling were performed twice, then the resin was washed with DMF (2×600 μl). Fmoc deprotection was performed using using 20% piperidine in DMF (450 μl, 2×2 min), and the resin was washed with DMF (7×600 μl). At the end of the peptide synthesis, the resin was washed with CH₂Cl₂ (2×600 μl) and resin beads were dried under suction.

### Reductive Release Procedure

Side-chain protecting group removal: After automated SPPS (5 μmol/well scale), the bottom of a 96-well synthesis plate was sealed by pressing the plate onto a soft 6 mm thick ethylene-vinyl acetate pad. The resin was incubated with TFA/TIPS/H₂O (300 μl, 95:2.5:2.5, v/v/v) for one hour covered by an adhesive PP plate lid. The TFA solution was discarded, and the resin was washed with CH₂Cl₂ (3×300 μl), and the procedure was repeated once. Reductive release: After air drying for at least an hour, a solution of 1,4-butanedithiol (BDT) and NEt₃ in DMF (both 100 mM, 200 μl, 4 equiv. relative to resin loading) was added to the resin and plates were agitated overnight at ambient temperature. The following day, the DMF solutions were pushed into a 96-well deep well plate via centrifugation (1000 rpm) and the reductive release procedure was repeated once for 5 h and unified into the same 96-well deep well plate. Upconcentration: A solution of TFA in milliQ-water (10% (v/v), 62 μl, 2 equiv. relative to NEts) was added to the wells, and the peptides were dried using a Speedvac concentrator (30° C., 1750 rpm, 0.1 mbar). Resolubilization and transfer: The dried peptide pellets were dissolved in DMSO (40 μl) and transferred to an Echo qualified 384-well PP source plate. Concentration determination: Ellman's assay was conducted to determine the concentrations of the di-thiol peptide stocks. Ellman's reagent5 (DTNB) was dissolved in assay buffer (150 mM NH₄HCO₃ in water:MeCN (90:10, v/v), pH 8) to a concentration of 10 mM. To a 384-well black microplate with transparent bottom was transferred dithiol peptide in DMSO (135 nl) by Echo using acoustic droplet ejection (ADE). Assay buffer (24 μl) was dispensed using CERTUS prior addition of DTNB solution (6 μl). Plates were centrifuged (400 g, 2 min) and absorbance (412 nm) was measured on a TECAN M200 plate reader. Concentration of di-thiol peptides were calculated using a previously recorded calibration curve:

$$abs = 0.287 \frac{mAU}{n mol} \cdot c + 0.0028$$

where: abs=absorbance at 412 nm in mAU

c=concentration of dithiol peptide

### Method Using Syringes

Side-chain protecting group removal: After automated SPPS (25 μmol scale), the fritted syringe containing the resin was incubated with TFA/TIPS/H₂O (4 ml, 95:2.5:2.5, v/v/v) for 2 h at ambient temperature. The TFA solution was discarded, and the resin was washed with CH₂Cl₂ (5×4 ml) and DMF (4 ml). Reductive release: After air drying for at least 1 h, a solution of BDT and NEts in DMF (both 100 mM, 2.0 ml, 4 equiv. relative to resin loading) was added to the syringe, which was agitated overnight at ambient temperature. The following day, the DMF solutions were pushed into a 50 ml conical falcon tube. Upconcentration: A solution of TFA in milliQ-water (10% (v/v), 312 μl, 2 equiv. relative to NEt₃) was added to peptide solution, which was dried using a Speedvac concentrator (30° C., 1750 rpm, 0.1 mbar) to afford the crude linear dithiol peptide ready for immediate cyclization in the next step.

### Macrocyclization Procedure

### Method Using Plates

Transfer to microtiter plates: Based on the determined concentration of each individual di-thiol peptide in DMSO, 40 nmol of dithiol peptides in DMSO were transferred into 384PP plates (one plate per linker) using ADE. Peptide reduction: As dithiol peptides oxidise over time in DMSO over time, it was ensured that the peptides were fully reduced by adding a solution of BDT and NEts in DMF (both 100 mM, 20 μl) to each well, followed by incubation for 30 min at ambient temperature. A solution of TFA in milliQ-water (10% (v/v), 6 μl, 2 equiv. relative to NEt₃) was added to each wells, and the peptides were dried using a Speedvac concentrator (30° C., 1750 rpm, 0.1 mbar) to afford fully reduced dithiol peptide pellets. Cyclization: Biselectrophilic linkers (L1-L7) were dissolved in a degassed 60 mM solution of NH₄HCO₃ in MeCN:H₂O (1:1 (v/v), pH 8) to a final concentration of 4 mM. The prepared linker solutions (40 μl, 4 equiv. relative to dithiol peptide) was added to the 384PP plates using a liquid dispenser, which were sealed with adhesive PP lids and agitated for 2 h at ambient temperature. Linker quenching: β-mercaptoethanol (β-ME) was dissolved in the prepared cyclization buffer to a final concentration of 32 mM. The prepared solution (20 μl, 4 equiv. relative to linker) was added to each well and incubated for 1 h at ambient temperature without plate lids. Upconcentration and resolubilization: Solvent was removed using a Speedvac concentrator (40° C., 1750 rpm, 0.1 mbar) to afford the peptide macrocycles as pellets, which were dissolved in DMSO (10 μl) and transferred to 384LDV plates to afford 4 mM macrocyclic peptide libraries that could immediately be applied in subsequent protease screening assays.

### Method in Conical Tubes

Cyclization: Biselectrophilic linker were dissolved in a degassed 60 mM solution of NH₄HCO₃ in MeCN:H₂O (1:1 (v/v), pH 8) to a final concentration of 4 mM. The prepared linker solutions (12.5 ml, 2 equiv. relative to dithiol peptide) was added to a conical tube containing the desired dithiol peptide pellet, and the solution was agitated for 2 h at ambient temperature. Linker quenching: Upon reaction completion (determined by LCMS), excess linker was quenched by addition of β-ME (14 μl, 200 μmol, 4 equiv. relative to linker) was added to the conical flask and agitated for at least 1 h prior to subsequent purification. Macrocycle purification: Samples were purified by preparative HPLC equipped with a C18 RP Waters OBD column. A linear gradient of solvent B (0.1% TFA in MeCN) over solvent A (0.1% TFA in water) rising linearly from 15% to 60% during t=2.00-32.00 min was applied at a flow rate of 14.0 ml/min. Pure fractions containing the desired product were unified and lyophilized to afford the products as colorless fluffy materials. DMSO stocks: Purified macrocycles were transferred into eppendorph tubes and DMSO was added to afford 5 mM or 20 mM compound stocks.

### Biochemical Assays

### Protease Screens of Macrocyclic Library

Enzyme inhibition of compound libraries was assessed by measuring the residual enzyme activity in presence of cyclic peptides (10 μM average concentration for thrombin, 20 μM average concentration for FXI, FXII, KLK5 and PKaI) at 1% final DMSO concentration. Crude macrocyclic libraries (4 mM DMSO stocks in 384-well LDV plates were transferred into 1536-well microtiter OptiPlates via ADE. Applied buffered solutions were prepared by filtration through PTFE syringe filters (0.22 μm) and assays were initiated by addition of protease (4.41 μl/well) in appropriate buffer (see list below) supplemented with bovine serum albumin (BSA; 0.1% w/v) and dispensed using a CERTUS automated liquid handler. Plates were incubated for 10 min at ambient temperature before fluorogenic substrate in appropriate buffer (4.5 μl) was added using a CERTUS automated liquid handler. Plates were centrifugated (800 g, 2 min) and fluorescence intensity was measured using a PHERAstar plate reader (excitation 384 nm, emission 440 nm) in time increments of 150 s over 15 min. Slopes of fluorescence increase (m) were calculated with Microsoft Excel (vers. 16.56). Negative controls were prepared without macrocycle. An average of 12 negative controls was used to calculate residual activities using Equation I below:

$$residual activity ( \% ) = \frac{{m}_{sample}}{{m}_{DMSOcontrol}} \cdot 100 \qquad Equation I$$

### Applied Buffer Compositions and Enzyme Concentrations

| | thrombin | | FXIa | | FXII | | KLK5 | | PKal | |
|---|---|---|---|---|---|---|---|---|---|---|
| enzyme conc. | 4 | nM | 0.25 | nM | 4 | nM | 7 | nM | 0.25 | nM |
| substrate | Cbz-G-G-R-AMC | | Boc-F-S-R-AMC | | Boc-Q-G-R-AMC | | Boc-V-P-R-AMC | | Cbz-F-R-AMC | |
| Bachem cat. # | 4002155 | | 4012340 | | 4016429 | | 4003460 | | 4003379 | |
| substrate conc. | 50 | μM | 50 | μM | 50 | μM | 100 | μM | 50 | μM |
| substrate K_{M} | 305 ± 46 | μM | n.d. | | 260 ± 40 | μM | 200 ± 10 | μM | 120 ± 28 | μM |
| | Buffer composition | | | | | | | | | |
| Tris-HCl pH 7.4 | 50 | mM | 50 | mM | 50 | mM | 50 | mM | 50 | mM |
| NaCl | 150 | mM | 150 | mM | 150 | mM | 100 | mM | 150 | mM |
| MgCl₂ | 10 | mM | 10 | mM | 10 | mM | — | | 10 | mM |
| CaCl₂ | 1 | mM | 1 | mM | 1 | mM | 10 | mM | 1 | mM |

| | | | | | |
|---|---|---|---|---|---|
| Triton-X | 0.01% | 0.01% | 0.01% | 0.01% | 0.01% |

### Identification of Active Species in Crude Macrocyclic Products from Hits

Selected hits from the library screening (Cmpds 195_L6, 237_L6 & 293L6) were re-synthesized and cyclized (40 nmol scale). Dried macrocyclic product was dissolved in MeCN:H₂O (1.5 ml, 1:1, v/v) and fractionated on a Thermo Fisher Dionex UltiMate 3000 system using a C18 NovaPak reversed phase column (10×150 mm, 125 Å pore, 5 μm particle). A linear gradient of solvent B (0.1% TFA in MeCN) over solvent A (0.1% TFA in water) rising linearly from 0% to 80% (for thrombin hits) or 0% to 95% (for PKaI hit) during t=2.00-22.0 min was applied at a flow rate of 4.00 mL/min. Fractions (one fraction/min) were collected in collection tubes and solvent was removed using a SpeedVac concentrator (30° C., 1750 rpm, 0.1 mbar). The dried content was redissolved in DMSO (50 μl), transferred to a 384-well PP source plate and dried using a SpeedVac concentrator (30° C., 1750 rpm, 0.1 mbar). Fractions were redissolved in DMSO (5 μl for thrombin, 2 μl for PKaI) and subsequent assays were conducted in black 384-well polystyrene plates with transparent bottom. DMSO fraction solution (0.5 μl) was pipetted to the microtiter plate and appropriate enzyme buffer solution (49.5 μl; similar composition as described previous page, technically using 2 nM thrombin) was added and incubated for 10 min at ambient temperature. Substrate in buffer (25 μl, similar composition as described previous page) was added, plates were centrifuged (800 g, 2 min) and fluorescence intensity was measured using a PHERAstar plate reader (excitation 384 nm, emission 440 nm) in time increments of 150 s over 15 min. Slopes of fluorescence increase (m) were calculated with Microsoft Excel (vers. 16.56). Negative controls were prepared without DMSO (0.5 μl) instead of fraction sample. An average of 6 negative controls was used to calculate residual activities using Equation I.

### IC₅₀ Determination

The half maximal inhibitor concentration (IC₅₀) values were determined from protease inhibition in a similar assay as library screening was conducted. Fold-dilution series of purified macrocyclic compounds were prepared in 384-well LDV source plates and transferred into 1536-well OptiPlates using ADE (final volume: 45 nl macrocycle/DMSO). Enzyme solution in buffer (4.5 μl) was added using Certus and incubated for 10 min. Subsequently, substrate in buffer (4.5 μl) was added and plates were centrifuged (700 g, 2 min) and fluorescence intensity was measured using a PHERAstar plate reader (excitation 384 nm, emission 440 nm) in time increments of 150 s over 15 min. Slopes of fluorescence increase (m) were calculated with Microsoft Excel (vers. 16.56). Negative controls were prepared without macrocycle. An average of 12 negative controls was used to calculate residual activities using Equation I. IC₅₀ values were obtained by fitting the resulting data to the concentration-response equation [constraints?] using GraphPad Prism (version 6.0.1) and Kᵢ values were calculated based on the IC₅₀ utilizing the Cheng-Prusoff equation¹¹:

$${K}_{i} = \frac{{IC}_{50}}{1 + \frac{{[ S ]}_{0}}{{K}_{M}}}$$

where [S]₀ is the initial substrate concentration and KM is the Michaelis-Menten constant¹² for the enzyme and substrate.

1. A method for preparing a library of peptides or an isolated peptide comprising
(a) releasing from a solid phase one or more linear dithiol peptides carrying a sulfhydryl group in the N-terminal region of the one or more peptides and being immobilized via a disulfide bridge in the C-terminal region of the one or more peptides on the solid phase by
(i) an agent reducing the disulfide bridge, thereby releasing the one or more linear dithiol peptides from the solid phase, wherein the agent is volatile and is removable by evaporation, or
(ii) a base that deprotonates the sulfhydryl group in the N-terminal region of the one or more linear dithiol peptides, thereby inducing an intramolecular disulfide exchange thereby releasing the one or more linear dithiol peptides from the solid phase in the form of one or more cyclic peptides.

2. The method of claim 1, further comprising after step (a) the step of removing the agent by evaporation.

3. The method of claim 1, further comprising step (b) of cyclizing the one or more linear dithiol peptides being released by the agent.

4. The method of claim 3, wherein the one or more dithiol peptides are cyclized by at least one bis-electrophilic reagent or by disulfide oxidation.

5. The method of claim 1, wherein the disulfide bonds of the one or more cyclic peptides of item (ii) are reduced and the peptides are recyclized by a bis-electrophilic reagent.

6. The method of claim 1, wherein the agent is selected from 1,3-propanedithiol, 1,4-butanedithiol, 2,4-pentanedithiol, ethane-1-thiol, propane-1-thiol, butane-1-thiol, propane-2-thiol, 2-methyl-1-propanethiol, butane-2-thiol, 2-methylpropane-2-thiol, 2-hydroxy-1-ethanethiol, 1,2-ethanedithiol, 2-propene-1-thiol, 3-methyl-1-butanethiol, thiophenol, benzylthiol, 2-butene-1-thiol, 3-butene-1-thiol, 2-methyl-2-propene-1-thiol and 3-methyl-2-butene-1-thiol, and is preferably 1,3-propanedithiol, 1,4-butanedithiol or 2,4-pentanedithiol and is most preferably 1,4-butanedithiol (BDT)
and/or
the base is selected from a tertiary, secondary or primary amine, a boron, aluminium or silicon hybride, and a base with an oxygen, nitrogen or carbon anion, and is preferably a tertiary, secondary or primary amine, more preferably a tertiary amine, even more preferably a tri-alkyl amine and most preferably N,N-diisopropylethylamine (DIPEA).

7. The method of claim 1, comprising prior to step (a) step (a′) the synthesis of the linear dithiol peptides on the solid phase.

8. The method of claim 1, wherein the side chains of the amino acids of the linear dithiol peptides are protected by protecting groups and the method further comprises prior to step (a) and after step (a′) the removal of the protecting groups while the linear dithiol peptides are immobilized on the solid phase.

9. The method of claim 1, wherein at least some of the linear dithiol peptides comprise a primary or secondary amine and the method further comprises modifying the primary or secondary amine with a carboxylic acid, wherein the cyclic peptides comprising a primary or secondary amine and the carboxylic acids are transferred by acoustic dispensing.

10. The method of claim 1, further comprising
(c) contacting the peptide library preferably without prior purification of the peptide library with a target molecule, and
(d) screening the peptide library for a peptide binding to and preferably inhibiting the target molecule.

11. The method of claim 10, wherein steps (c) and (d) are carried out in the same wells in which the primary or secondary amine has been modified with a carboxylic acid.

12. The method of claim 10, wherein the target molecule is a protein, peptide, nucleic acid molecule, carbohydrate or fatty acid, and is preferably a protein or peptide.

13. The method of claim 1, wherein the solid phase comprises a resin.

14. The method of claim 1, wherein the
linear dithiol peptides comprise
linear dithiol peptides having a molecular weight of less than 1000 Da and preferably of
less than 600 Da, and/or
linear dithiol peptides comprising 3 or 4 amino acids and preferably 3 amino acids.

15. A method for the diversification of a macrocyclic compound library,
preferably a cyclic peptide library, wherein at least some macrocyclic compounds,
preferably cyclic peptides comprise a primary or secondary amine, wherein the method comprises modifying the primary or secondary amine with a carboxylic acid.

16. The method of claim 13, wherein the resin is an apolar resin.

17. The method of claim 13, wherein the resin is a polystyrene resin.
